(19) United States
(12) Patent Application Publication
(10) Pub. No.: US 2025/0017878 A1
(43) Pub. Date: Jan. 16, 2025

(54) USE OF NADOLOL TO TREAT CHRONIC OBSTRUCTIVE PULMONARY DISEASE BY BLOCKAGE OF THE ARRESTIN-2 PATHWAY

(71) Applicant: Chronic Airway Therapeutics Limited, East Kew, VIC (AU)

(72) Inventor: Mitchell Glass, Wilmington, DE (US)

(21) Appl. No.: 18/281,135

(22) PCT Filed: Mar. 8, 2022
(86) PCT No.: PCT/US22/19336

§ 371 Date: Sep. 8, 2023

Related U.S. Application Data
(60) Provisional application No. 63/158,701, filed on Mar. 9, 2021.

(30) Foreign Application Priority Data
Mar. 9, 2021 (US) 63158701

(51) Int. Cl. A61K 31/138; A61K 45/06; A61P 11/06

(52) U.S. Cl. CPC A61K 31/138; A61K 45/06; A61P 11/06

(57) ABSTRACT

The present invention is directed to compositions and methods for treating chronic obstructive pulmonary disease (COPD) and other diseases and conditions affecting the respiratory tract through inhibition of the β-arrestin (arrestin-2) pathway by use of β-adrenergic inverse agonists, particularly including nadolol. The compositions and methods can be used to treat pulmonary symptoms associated with infection with SARS-CoV-2.

### CROSS-REFERENCES TO RELATED APPLICATION

This application claims the benefit of U.S. Provisional Patent Application Ser. No. 63/158,701 by Dr. Mitchell Glass, entitled “Use of Nadolol to Treat Chronic Obstructive Pulmonary Disease by Blockage of the Arrestin-2 Pathway,” and filed on Mar. 9, 2021, the contents of which are incorporated herein in their entirety by this reference.

### FIELD OF THE INVENTION

This invention is directed to the use of nadolol to treat chronic obstructive pulmonary disease (COPD) and other diseases and conditions affecting the respiratory tract through inhibition of the β-arrestin (arrestin-2) pathway, including methods and compositions for inhibition of the β-arrestin pathway.

### BACKGROUND OF THE INVENTION

Chronic obstructive pulmonary disease (COPD) is a type of obstructive lung disease characterized by long-term breathing problems and poor airflow. The main symptoms of COPD include shortness of breath and cough with sputum production; the cough is typically chronic. COPD is normally a progressive disease, meaning that it worsens over time. Eventually, for COPD patients, everyday activities, even activities such as getting dressed or walking, become difficult. COPD is a major cause of morbidity and mortality in both developed countries and developing countries.

COPD is divided into chronic bronchitis and emphysema and the combination of those two conditions. Chronic bronchitis is one of several conditions that are marked by epithelial changes which may include loss of ciliated epithelial cells, an increased number of glands, and an increased number of goblet cells, causing both increased mucus production and abnormal mucus production. These conditions include, but are not limited to, chronic bronchitis with or without airway obstruction and chronic bronchitis with or without emphysema.

COPD is a major public health problem. In 2020, COPD is projected to rank fifth worldwide in terms of burden of disease and third in terms of mortality. The prevalence and burden of COPD are expected to increase in the coming decades due to continued exposure to COPD risk factors and the aging of the world's population. Morbidity measures traditionally include physician visits, emergency department visits, and hospitalizations. Morbidity due to COPD increases with age and may be affected by other comorbid chronic conditions (e.g., cardiovascular disease, musculoskeletal impairment, or diabetes mellitus) that are frequent in patients with COPD and may impact on the patient's health status, as well as interfere with COPD management. The increased mortality of COPD is largely driven by the continued prevalence of smoking in many populations and subgroups of populations, reduced mortality from other common causes of death, and aging of the world population. COPD is also associated with significant economic burden. There is a direct relationship between the severity of COPD and the cost of care, and the distribution of costs changes as the disease progresses. For example, hospitalization and ambulatory oxygen costs soar as COPD severity increases. In developing countries, direct medical costs may be less important than the impact of COPD on workplace and home productivity. In 1990, COPD was the 12th leading cause of disability-adjusted life years (DALYs) lost in the world, responsible for 2.1% of the total. According to the projections, COPD will be the seventh leading cause of DALYs lost worldwide in 2030.

Chronic bronchitis is particularly significant as a health risk in China, a country with an extremely high rate of cigarette smoking. In this context, with respect to China, the use of the diagnosis COPD is reserved for ex-smokers. Chronic bronchitis is the broader term and includes COPD as well as equivalent pathology occurring in patients who have never smoked and current smokers. COPD impacts 13.7% of the population in China over the age of 40. COPD is one of the three top causes of death in China. The direct medical costs in China range from US $72-$3565 per capita (33%-118% of average income). COPD is responsible for 12 million hospitalizations each year and 1.2 billion doctor visits per year in China.

The most common symptoms of COPD are sputum production, a productive cough, and shortness of breath. These symptoms are present for a prolonged period of time and typically worsen over time. A chronic cough is often the first symptom to develop. Early on in the course of the disease it may just occur occasionally or may not result in sputum. When a cough persists for more than three months each year for at least two years with sputum production and without another explanation for the cough, it is by definition chronic bronchitis. Most chronic bronchitis is secondary to cigarette smoking, a syndrome that is closely related to COPD that may or may not present with the reduced airflow typical of COPD. The amount of sputum produced can change over periods of hours or days. In some cases, the cough may not be present or may occur occasionally or may not be productive. In some cases, vigorous coughing can lead to a brief loss of consciousness, loss of bladder control, or to rib fractures. Patients with COPD frequently have a history of episodes of coryza (the “common cold”) that last for abnormally long periods of time.

In COPD, shortness of breath is frequently the symptom that bothers patients the most. Typically, the shortness of breath is worse on prolonged exertion and worsens over time. In the advanced stages of COPD or end-stage pulmonary disease, it occurs during rest and may always be present. Shortness of breath is a source of anxiety and poor quality of life for COPD patients. In COPD, breathing out (exhalation) may take longer than breathing in (inhalation). Chest tightness may occur, but is not a common symptom for COPD. COPD patients with obstructed airflow may have wheezing, rhonchi (bronchial breath sounds) or decreased sounds on inhalation or exhalation that are audible on examination of the chest with a stethoscope. A barrel chest may occur in COPD patients, but is relatively uncommon. Tripod positioning to assist breathing may occur as the disease progresses.

Advanced COPD leads to high pressure on the lung arteries, which in turn strains the right ventricle of the heart. This condition is referred to as cor pulmonale, which can lead to symptoms of leg swelling and bulging neck veins. COPD is the most frequent cause of cor pulmonale.

COPD often occurs along with one or more additional conditions, due to shared risk factors. These additional conditions include ischemic heart disease, hypertension, diabetes mellitus (typically type 2 diabetes), muscle wasting, osteoporosis, lung cancer, anxiety disorder, sexual dysfunction, and depression. Tiredness and fatigue are common in patients with advanced disease. Fingernail clubbing may occur, but is not specific to COPD.

An acute exacerbation of COPD is defined as increased shortness of breath, increased sputum production, a change in the color of the sputum from clear to green or yellow, or an increase in the frequency or intensity of cough in a patient who had been diagnosed with COPD. Exacerbations may present with signs of increased breathing effort such as fast breathing, a fast heart rate, sweating, active use of muscles in the neck, a bluish tinge to the skin, and, in very severe exacerbations, confusion or combative behavior. Crackles (rales) also may be heard over the lungs on examination with a stethoscope.

The primary cause of COPD in developed nations is tobacco smoke. In some countries, occupational exposure and pollution from indoor fires may be causes of COPD. Typically, exposure to the causes of COPD must occur for an extended period of time, often several decades, before symptoms of COPD develop. Genetic factors also play a role. Genetic factors associated with the occurrence or severity of COPD include, but are not limited to, a hereditary deficiency of alpha-1 antitrypsin (AATD) (J. K. Stoller & L. S. Aboussousan, “Alpha1-Antitrypsin Deficiency,” Lancet 365: 2235-2236 (2005)). Single genes, such as the gene encoding matrix metalloproteinase 12 (MMP-12), and glutathione S-transferase have been related to a decrease in lung function or the risk of COPD. Several genome-wide association studies have linked a number of genetic loci with COPD (or with FEV₁ or FEV₁/FVC as the phenotype) including markers near the alpha-nicotinic acetylcholine receptor, hedgehog interacting protein (HHIP), and several others.

Patients with asthma or airway hyperreactivity are at increased risk of developing COPD. Patients with infectious diseases such as tuberculosis or AIDS are also at greater risk of developing COPD.

Acute exacerbations of COPD are typically triggered by infections, which can be bacterial or viral, exposure to environmental pollution, and, in some cases, exposure to cold. Patients with more severe underlying disease have more frequent exacerbations. Additionally, patients with COPD may experience pulmonary embolisms.

With respect to the pathophysiology of COPD, COPD is a type of obstructive lung disease in which chronic, incompletely reversible poor airflow (airflow limitation) and inability to breathe out fully (air trapping) exist. The poor airflow is the result of breakdown of lung tissue (known as emphysema), and small airways disease known as obstructive bronchiolitis. The relative contributions of these two factors vary between people. Severe destruction of small airways can lead to the formation of large focal lung pneumatoses, known as bullae, that replace lung tissue. This form of disease is called bullous emphysema.

COPD develops as a significant and chronic inflammatory response to inhaled irritants. Chronic bacterial infections may also add to this inflammatory state. The inflammatory cells involved include neutrophil granulocytes and macrophages, two types of white blood cells. Those who smoke additionally have Tc₁ lymphocyte involvement and some people with COPD have eosinophil involvement similar to that in asthma. Part of this cell response is brought on by inflammatory mediators such as chemotactic factors. Other processes involved with lung damage include oxidative stress produced by high concentrations of free radicals in tobacco smoke and released by inflammatory cells, and breakdown of the connective tissue of the lungs by proteases that are insufficiently inhibited by protease inhibitors. The destruction of the connective tissue of the lungs leads to emphysema, which then contributes to the poor airflow, and finally, poor absorption and release of respiratory gases. General muscle wasting that often occurs in COPD may be partly due to inflammatory mediators released by the lungs into the blood. Narrowing of the airways occurs due to inflammation and scarring within them. This contributes to the inability to breathe out fully. The greatest reduction in air flow occurs when breathing out, as the pressure in the chest is compressing the airways at this time. This can result in more air from the previous breath remaining within the lungs when the next breath is started, resulting in an increase in the total volume of air in the lungs at any given time, a process called hyperinflation or air trapping. Hyperinflation from exercise is linked to shortness of breath in COPD, as breathing in is less comfortable when the lungs are already partly filled. Hyperinflation may also worsen during an exacerbation.

Some patients with COPD also have a degree of airway hyperresponsiveness to irritants similar to that found in asthma patients.

Low oxygen levels, and eventually, high carbon dioxide levels in the blood, can occur from poor gas exchange due to decreased ventilation from airway obstruction, hyperinflation, and a reduced desire to breathe. During exacerbations, airway inflammation is also increased, resulting in increased hyperinflation, reduced expiratory airflow, and worsening of gas transfer. This can also lead to insufficient ventilation, and eventually low blood oxygen levels. Low oxygen levels, if present for a prolonged period, can result in narrowing of the arteries in the lungs, while emphysema leads to breakdown of capillaries in the lungs. Both of these changes result in increased blood pressure in the pulmonary arteries, which may cause cor pulmonale.

The diagnosis of COPD should be considered in anyone over the age of 35 to 40 who has shortness of breath, a chronic cough, sputum production, or frequent winter colds and a history of exposure to risk factors for the disease. Spirometry is then used to confirm the diagnosis. Spirometry measures the amount of airflow obstruction present and is generally carried out after the use of a bronchodilator, a medication to open up the airways. Two main components are measured to make the diagnosis, the forced expiratory volume in one second (FEV₁), which is the greatest volume of air that can be breathed out in the first second of a breath, and the forced vital capacity (FVC), which is the greatest volume of air that can be breathed out in a single large breath. Normally, 75-80% of the FVC comes out in the first second and a FEV₁/FVC ratio less than 70% in someone with symptoms of COPD defines a person as having the disease.

With respect to the treatment of COPD, no cure for COPD is known, but its symptoms are treatable and its progression can be delayed. The major goals of management are to reduce risk factors, manage stable COPD, prevent and treat acute exacerbations, and manage associated illnesses. The only measures that have been shown to reduce mortality are smoking cessation and supplemental oxygen. Stopping smoking can substantially reduce the death rate from COPD. Other measures that have been used or suggested in the management or treatment of COPD include vaccinations against influenza viruses, vaccinations against pneumococcal bacteria, vaccinations against Haemophilus influenzae, exercise programs, and weight management.

A number of medications have been used in attempts to treat or manage COPD. In general, these medications may provide significant relief of symptoms but do not treat the underlying disease. The use of these medications is distinct from the management measures described above, including the possible use of medications such as nicotine (as nicotine replacement therapy), buproprion, or varenicline.

Inhaled bronchodilators are the primary medications used. The two major types of bronchodilators are the 2-agonists and the anticholinergics. Both of these types of bronchodilators exist in short-acting or long-acting forms. In COPD patients with mild disease, short-acting agents are generally recommended on an as-needed basis. In COPD patients with more severe disease, long-acting agents are generally recommended; long-acting agents partly work by reducing hyperinflation. If long-acting bronchodilators are insufficient, then inhaled corticosteroids are typically added.

Short-acting β₂-agonists include salbutamol, fenoterol, levalbuterol, and terbutaline. Long-acting β₂-agonists (LABAs) include salmeterol, formoterol, arformeterol, indacaterol, tulobuterol, and vilanterol. Anticholinergics include ipratropium bromide, oxitropium bromide, and tiotropium. Ipratropium bromide and oxitropium bromide are short-acting anticholinergics, while tiotropium is a long-acting anticholinergic. Other anticholinergic agents include the long-acting muscarinic antagonists (LAMAs) aclidinium bromide, umeclidinium bromide, glycopyrronium bromide, and glycopyrronium tosylate.

Corticosteroids used for treatment of COPD include triamcinolone acetonide, fluticasone propionate, beclomethasone, budesonide, ciclesonide, flunisolide, mometasone, prednisone, and methylprednisolone. Although corticosteroids are generally used in inhaled form, they can also be administered orally such as by ingestion of pills; in particular, prednisone and methylprednisolone are typically administered orally.

Various therapeutic combinations have been tried, including combinations of agents from two categories of agents (β₂-agonists, anticholinergics, and corticosteroids), as well as combinations of agents from all three categories of agents.

Still other categories of medications can be or have been used in COPD. These additional categories of agents include methylxanthines, antibiotics, phosphodiesterase 4-inhibitors, leukotriene antagonists, mucolytic agents, and mast cell stabilizers.

Methylxanthines include theophylline, theobromine, aminophylline, IBMX (3-isobutyl-1-methylxanthine), paraxanthine, and pentoxifylline.

Antibiotics that can be used to treat COPD or, more preferably, secondary infections associated with COPD, including antibiotics of the macrolide group, including erythromycin, azithromycin, clarithromycin, fidaxomicin, and telithromycin.

Phosphodiesterase-4 inhibitors include roflumilast, cilomilast, and ibudilast.

Leukotriene antagonists include montelukast, pranlukast, and zafirlukast.

Mucolytic agents include carbocysteine, N-acetylcysteine, ambroxol, bromhexine, erdosteine, mecysteine, iodinated glycerol, and recombinant human DNase.

Mast cell stabilizers include cromoglicic acid, ketoxifen, olopatadine, rupatadine, mepolizumab, omalizumab, pemirolast, azelastine, and tranilast.

However, despite the range of medications available for the treatment of COPD and its sequelae and complications, there is still an urgent need for improved treatments for COPD. In particular, there is a need for treatments for COPD that can modulate the underlying disease process and not merely treat the symptoms.

Additionally, other pulmonary airway diseases and conditions have similar symptoms and courses and are subject to similar treatments; such additional pulmonary airway diseases and conditions include, but are not limited to: asthma, particularly moderate or severe asthma, bronchiectasis, bronchitis, Churg-Strauss syndrome, pulmonary sequelae of cystic fibrosis, emphysema, allergic rhinitis, pneumonia, and pulmonary symptoms associated with infection with SARS-CoV-2 (R. C. Boucher, “Muco-Obstructive Lung Diseases,” New Engl. J. Med. 380: 1941-1953 (2019)). The emergence of SARS-CoV-2 is of great significance for a number of reasons. Patients with COPD are extremely susceptible to infection with SARS-CoV-2, and such patients tend to have much more severe disease, with increased risk of death. Therefore, there is an urgent need for improved methods for treatment of these additional respiratory or respiratory-related conditions, particularly infection with SARS-CoV-2.

### SUMMARY OF THE INVENTION

Improved treatment methods and compositions for treatment of COPD and other respiratory or respiratory-related conditions as described above, including SARS-CoV-2, are based on the activity of nadolol, a non-specific β-blocker with β-adrenergic inverse agonist activity, in blocking the β-arrestin (arrestin-2) pathway, particularly in blocking signaling at airway epithelial cell β₂ receptors. The terms “β-arrestin” and “arrestin-2” are used interchangeably herein, and refer to the same protein and pathway mediated by that protein.

One aspect of the present invention is a method for treatment of pulmonary airway disease in a subject suffering from pulmonary airway disease comprising administration of a therapeutically effective quantity of nadolol or a derivative or analog of nadolol to inhibit the β-arrestin pathway to treat the pulmonary airway disease. Typically, the pulmonary airway disease is selected from the group consisting of chronic obstructive pulmonary disease (COPD), asthma, bronchiectasis, bronchitis, Churg-Strauss syndrome, pulmonary sequelae of cystic fibrosis, emphysema, allergic rhinitis, pneumonia, and pulmonary symptoms associated with infection with SARS-CoV-2. Preferably, the pulmonary airway disease is COPD.

Typically, the method comprises administration of a therapeutically effective quantity of nadolol. Typically, the nadolol is the RSR stereoisomer of nadolol.

In one alternative, the method exerts a therapeutic effect that is an upregulation of pulmonary β₂-adrenergic receptors. Typically, the method exerts a therapeutic effect that is increased pulmonary airway relaxation responsiveness to β₂-adrenergic agonist drugs. Typically, the method exerts a therapeutic effect that is a reversal of mucous metaplasia and mucus cell metaplasia.

Typically, the nadolol or the derivative or analog of nadolol is administered by a route selected from the group consisting of oral, sustained-release oral, parenteral, sublingual, buccal, administration by insufflation, and administration by inhalation. Typically, the administration of the nadolol or the derivative or analog of nadolol is performed by dose titration over time in a series of graduated doses starting from the lowest dose and increasing to the highest dose. Typically, when the highest dose is reached, the nadolol or the derivative or analog of nadolol continues to be administered at that dose. Preferably, the nadolol or the derivative of nadolol is administered by the inhaled route or the oral route. More preferably, the nadolol or the derivative of nadolol is administered by the inhaled route.

Typically, the method of sustained-release oral administration of the nadolol or the derivative or analog of nadolol results in continuous levels of the nadolol or the derivative or analog of nadolol in the bloodstream. Typically, the method of administering the nadolol by inhaled administration comprises administration of a dose administered by pressurized meter dose inhaler (pMDI), dry powder inhaler, or nebulizer in doses that either do or not generate measurable blood levels in the range typically associated with oral dosing. Typically, the inhaled dose will be delivered by pMDI and will be in the range of from about 1% to about 10% of the minimally effective oral dose.

Typically, the inhibition of β-arrestin prevents or reverses the desensitization of β₂-adrenergic receptors. Typically, the inhibition of β-arrestin also prevents or reverses the internalization of β₂-adrenergic receptors. Typically, the inhibition of β-arrestin prevents or reverses phosphorylation of β₂-adrenergic receptors by a second-messenger-specific protein kinase or a specific G-protein-coupled receptor kinase. Typically, the inhibition of β-arrestin also prevents or reverses degradation of a second messenger by a scaffolding phosphodiesterase.

In one alternative, the method further comprises administration of a therapeutically effective quantity of a β₂-selective adrenergic agonist. Typically, the β₂-selective adrenergic agonist is selected from the group consisting of albuterol, arfomoterol, bambuterol, bitolterol, broxaterol, buphenine, carbuterol, clenbuterol, clorprenaline, colterol, dobutamine, fenoterol, formoterol, isoetharine, isoprenaline, levabuterol, levosalbutamol, mabuterol, metaprotenerol, methoxyphenamine, pirbuterol, procaterol, ractopamine, reproterol, ritodrine, salmeterol, terbutaline, zilpaterol, and the salts, solvates, and prodrugs thereof.

In another alternative, the method further comprises administration of a therapeutically effective quantity of a corticosteroid. Typically, the corticosteroid is selected from the group consisting of AZD-5423 (2,2,2-trifluoro-N-[(1R,2S)-1-{[1-(4-fluorophenyl)-1H-indazol-5-yl]oxy}-1-(3-methoxyphenyl)-2-propanyl]acetamide), beclomethasone, budesonide, ciclesonide, deflazacort, flunisolide, fluticasone, methylprednisolone, mometasone, prednisolone, prednisone, dexamethasone, and triamcinolone, and the salts, solvates, and prodrugs thereof.

In yet another alternative, the method further comprises administration of a therapeutically effective quantity of an anticholinergic drug. Typically, the anticholinergic drug is selected from the group consisting of ipratropium bromide, tiotropium bromide, oxitropium bromide, abediterol, aclidinium bromide, glycopyrronium bromide, umeclidinium bromide, and the salts, solvates, and prodrugs thereof.

In yet another alternative, the method further comprises administration of a therapeutically effective quantity of at least one biological. Typically, the at least one biological is selected from the group consisting of an anti-IL4 antibody, an anti-IL13 antibody, an inhibitor of IL4 and IL13, an anti-IL5 antibody, and an anti-IL8 antibody.

In still another alternative, the method further comprises administration of a therapeutically effective quantity of a xanthine compound. Typically, the xanthine compound is selected from the group consisting of theophylline, extended-release theophylline, aminophylline, theobromine, enprofylline, diprophylline, isbufylline, choline theophyllinate, albifylline, arofylline, bamifylline, caffeine, 8-chlorotheophylline, diprophylline, doxofylline, enprofylline, etamiphylline, furafylline, 1-isobutyl-1-methylxanthine, proxyphylline, and xanthinol, and the salts, solvates, and prodrugs thereof.

In yet another alternative, the method further comprises administration of a therapeutically effective quantity of an anti-IgE antibody. Typically, the anti-IgE antibody is a monoclonal antibody or a genetically engineered antibody that is derived from a monoclonal antibody; the antibody can be humanized, such as omalizumab.

In yet another alternative, the method further comprises administration of a therapeutically effective quantity of a leukotriene antagonist. Typically, the leukotriene antagonist is selected from the group consisting of montelukast, pranlukast, and zafirlukast, and the salts, solvates, and prodrugs thereof.

In still another alternative, the method further comprises administration of a therapeutically effective quantity of a phosphodiesterase IV inhibitor. Typically, the phosphodiesterase IV inhibitor is selected from the group consisting of roflumilast, cilomilast, piclamilast, and ibudilast, and the salts, solvates, and prodrugs thereof.

In yet another alternative, the method further comprises administration of a therapeutically effective quantity of a 5-lipoxygenase inhibitor. Typically, the 5-lipoxygenase inhibitor is selected from the group consisting of zileuton and fenleuton, and the salts, solvates, and prodrugs thereof.

In still another alternative, the method further comprises administration of a therapeutically effective quantity of a mast cell stabilizer. Typically, the mast cell stabilizer is selected from the group consisting of azelastine, cromoglicic acid, ketotifen, lodoxamide, nedocromil, olopatadine, and pemirolast, and the salts, solvates, and prodrugs thereof.

In yet another alternative, the method further comprises administration of a therapeutically effective quantity of an arrestin-2 inhibitor.

One alternative of an arrestin-2 inhibitor that can be used in methods according to the present invention is a protein fragment of arrestin-2.

Another alternative of an arrestin-2 inhibitor that can be used in methods according to the present invention is a compound of Formula (A-I) with alternatives for substituents as defined below.

Another alternative of an arrestin-2 inhibitor that can be used in methods according to the present invention is an omega-3 fatty acid selected from the group consisting of DHA (docosahexaenoic acid) and EPA (eicosapentaenoic acid).

Yet another alternative of an arrestin-2 inhibitor that can be used in methods according to the present invention is a CXCR2 inhibitor selected from the group consisting of SB225002 (N-(2-bromophenyl)-N′-(2-hydroxy-4-nitrophenyl)urea), AZD5069 (N-(2-((2,3-difluorobenzyl)thio)-6-(((2R,3S)-3,4-dihydroxybutan-2-yl)oxy)pyrimidin-4-yl)azetidine-1-sulfonamide); SB265610 (1-(2-bromophenyl)-3-(4-cyano-1H-benzo[d][1,2,3]triazol-7-yl)urea); navarixin; danirixin; CXCR2-IN-1 (1-(2-chloro-3-fluorophenyl)-3-[4-chloro-2-hydroxy-3-(1-methylpiperidin-4-yl)sulfonylphenyl]urea); SRT3109 (N-(2-((2,3-difluorobenzyl)thio)-6-((3,4-dihydroxybutan-2-yl)amino)pyrimidin-4-yl)azetidine-1-sulfonamide); and SRT3190 (N-[2-[(2,3-difluorophenyl)methylsulfanyl]-6-[[(2S,3R)-3,4-dihydroxybutan-2-yl]amino]pyrimidin-4-yl]azetidine-1-sulfonamide).

Still another alternative of an arrestin-2 inhibitor that can be used in methods according to the present invention is a MyD88 inhibitor. Typically, the MyD88 inhibitor is selected from the group consisting of ST2825 ((4R,7R,8aR)-1′-[2-[4-[[2-(2,4-dichlorophenoxy)acetyl]amino]phenyl]acetyl]-6-oxospiro[3,4,8,8a-tetrahydro-2H-pyrrolo[2,1-b][1,3]thiazine-7,2′-pyrrolidine]-4-carboxamide), and T6167923 (4-(3-bromophenyl)sulfonyl-N-(1-thiophen-2-ylethyl)piperazine-1-carboxamide).

Yet another alternative of an arrestin-2 inhibitor that can be used in methods according to the present invention is a MD2 inhibitor. Typically, the MD2 inhibitor is L48H37 ((3E,5E)-1-ethyl-3,5-bis[(2,3,4-trimethoxyphenyl)methylidene]piperidin-4-one).

Still another alternative of an arrestin-2 inhibitor that can be used in methods according to the present invention is inositol hexaphosphate (IP6).

Yet another alternative of an arrestin-2 inhibitor that can be used in methods according to the present invention is barbadin.

Still another alternative of an arrestin-2 inhibitor that can be used in methods according to the present invention is an inhibitor of protein kinase A. Typically, the inhibitor of protein kinase A is selected from the group consisting of: (i) H89 (N-[2-[[3-(4-bromophenyl)-2-propenyl]amino]ethyl]-5-isoquinolinesulfonamide dihydrochloride); (ii) N-(ω-undecylenoyl)phenylalanine; (iii) 3′,5′-cyclic monophosphorothioate-R; (iv) H-7 (5-(2-methylpiperazin-1-yl)sulfonylisoquinoline dihydrochloride); (v) H-9 (N-(2-aminoethyl)-5-isoquinolinesulfonamide; (vi) 6-22 amide; (vii) a protein kinase A inhibitor selected from the group consisting of: fasudil; N-[2-(phosphorylated bromonitroarginylamino)ethyl]-5-isoquinoline sulfonamide; 1-(5-quinolinesulfonyl)piperazine; 4-cyano-3-methylisoquinoline; acetamido-4-cyano-3-methylisoquinoline; 8-bromo-2-monoacyladenosine-3,5-cyclic monophosphorothioate; adenosine 3,5-cyclic monophosphorothioate; 2-O-monobutyl-cyclic adenosine monophosphate; 8-chloro-cyclic adenosine monophosphate; N-[2-(cinnamoylamino acid)]-5-isoquinolinone; reverse phase-8-hexylamino adenosine 3,5-monophosphorothioate; reverse phase-8-piperidinyladenosine-cyclic adenosine monophosphate; reverse phase-adenosine 3,5-cyclic monophosphorothioate; 5-iodotuberculin; 8-hydroxyadenosine-3,5-monophosphorothioate; calphostin C; daphnetin; reverse phase-8-chlorophenyl-cyclic adenosine monophosphate; reverse phase-cyclic adenosine monophosphate; reverse phase-8-Br-cyclic adenosine monophosphate; 1-(5-isoquinolinesulfonyl)-2-methylpiperidine; 8-hydroxyadenosine-3′,5′-monophosphate; 8-hexylaminoadenosine-3′,5′-monophosphate; and reverse phase-adenosine 3′,5′-cyclic monophosphate.

Yet another alternative of an arrestin-2 inhibitor that can be used in methods according to the present invention is a phospholipase C inhibitor. Typically, the phospholipase C inhibitor is selected from the group consisting of sodium aristolochate; D609 (sodium tricyclodecan-9-yl xanthogenate); D-erythro-dihydrosphingosine; U-73122 (1-(6-((17β-3-methoxyestra-1,3,5(10)-trien-17-yl)amino)hexyl)-1H-pyrrole-2,5-dione); pyrrolidinethiocarbamate; neomycin sulfate; thielavin B; edelfosine; heterocyclyl-substituted anilino phospholipase C inhibitors; DCIC (3,4-dichloroisocoumarin); and calporoside or derivatives of calporoside.

In still another alternative, the method further comprises administration of a therapeutically effective quantity of an inhibitor of a GRK. Typically, the inhibitor of the GRK is a nitric oxide donor that donates nitric oxide or a related redox species. The inhibitor of the GRK indirectly acts as an inhibitor of arrestin-2.

Another aspect of the present invention is a pharmaceutical composition comprising:
(1) a therapeutically effective quantity of nadolol or a derivative or analog of nadolol to inhibit the β-arrestin pathway to treat a pulmonary airway disease; and
(2) a pharmaceutically acceptable carrier.

Typically, the pulmonary airway disease treatable by administration of the pharmaceutical composition is selected from the group consisting of chronic obstructive pulmonary disease (COPD), asthma, bronchiectasis, bronchitis, Churg-Strauss syndrome, pulmonary sequelae of cystic fibrosis, emphysema, allergic rhinitis, pneumonia, and pulmonary symptoms associated with infection with SARS-CoV-2. Preferably, the pulmonary airway disease treatable by administration of the pharmaceutical composition is chronic obstructive pulmonary disease.

Typically, the pharmaceutical composition comprises a therapeutically effective quantity of nadolol.

Typically, administration of the pharmaceutical composition exerts a therapeutic effect that is an upregulation of pulmonary 2-adrenergic receptors. Typically, administration of the pharmaceutical composition exerts a therapeutic effect that is increased pulmonary airway relaxation responsiveness to 2-adrenergic agonist drugs. Typically, administration of the pharmaceutical composition exerts a therapeutic effect that is a reversal of mucous metaplasia and mucus cell metaplasia.

Typically, the pharmaceutical composition is formulated for administration by a route selected from the group consisting of oral, sustained-release oral, parenteral, sublingual, buccal, administration by insufflation, and administration by inhalation. Preferably, the pharmaceutical composition is formulated for administration by inhalation.

Typically, in pharmaceutical compositions according to the present invention, the nadolol or the derivative or analog of nadolol is nadolol.

Typically, administration of the pharmaceutical composition results in continuous levels of the nadolol or the derivative or analog of nadolol in the bloodstream when the composition is formulated for sustained-release oral administration.

Typically, when the composition comprises nadolol, the quantity of nadolol in the composition is selected from the group consisting of 1 mg, 3 mg, 5 mg, 10 mg, 15 mg, 30 mg, 50 mg, and 70 mg per unit dose.

In another alternative, the composition further comprises a therapeutically effective quantity of a β₂-selective adrenergic agonist. Suitable β₂-selective adrenergic agonists are as described above.

In yet another alternative, the composition further comprises a therapeutically effective quantity of a corticosteroid. Suitable corticosteroids are as described above.

In still another alternative, the composition further comprises a therapeutically effective quantity of an anticholinergic drug. Suitable anticholinergic drugs are as described above.

In yet another alternative, the composition further comprises a therapeutically effective quantity of a xanthine compound. Suitable xanthine compounds are as described above.

In still another alternative, the composition further comprises a therapeutically effective quantity of an anti-IgE antibody. Suitable anti-IgE antibodies are as described above.

In yet another alternative, the composition further comprises a therapeutically effective quantity of at least one biological. Suitable biologicals are as described above.

In yet another alternative, the composition further comprises a therapeutically effective quantity of a leukotriene antagonist. Suitable leukotriene antagonists are as described above.

In still another alternative, the composition further comprises a therapeutically effective quantity of a phosphodiesterase IV inhibitor. Suitable phosphodiesterase IV inhibitors are as described above.

In yet another alternative, the composition further comprises a therapeutically effective quantity of a 5-lipoxygenase inhibitor. Suitable 5-lipoxygenase inhibitors are as described above.

In still another alternative, the composition further comprises a therapeutically effective quantity of a mast cell stabilizer. Suitable mast cell stabilizers are as described above.

In yet another alternative, the composition further comprises a therapeutically effective quantity of an arrestin-2 inhibitor. Arrestin-2 inhibitors that can be included in compositions according to the present invention are as described above, and include, but are not limited to: a compound of Formula (A-I); an omega-3 fatty acid; a CXCR2 inhibitor; a MyD88 inhibitor; a MD2 inhibitor; a therapeutically effective quantity of an inhibitor of a GRK, such as, but not limited to, a nitric oxide donor that donates nitric oxide or a related redox species; inositol hexaphosphate (IP6); barbadin; an inhibitor of protein kinase A; or a phospholipase C inhibitor.

In compositions according to the present invention, the pharmaceutically acceptable carrier can be, but is not limited to, a pharmaceutically acceptable carrier selected from the group consisting of a solvent, a dispersion medium, a coating, an antibacterial agent, an antifungal agent, an isotonic agent, an absorption delaying agent, a preservative, a sweetening agent for oral administration, a thickening agent, a buffer, a liquid carrier, a wetting, solubilizing, or emulsifying agent; an acidifying agent, an antioxidant, an alkalinizing agent, a carrying agent, a chelating agent, a colorant, a complexing agent, a suspending or viscosity-increasing agent, a flavor or perfume, an oil, a penetration enhancer, a polymer, a stiffening agent, a protein, a carbohydrate, a bulking agent, and a lubricating agent.

### BRIEF DESCRIPTION OF THE DRAWINGS

These and other features, aspects, and advantages of the present invention will become better understood with reference to the following description, appended claims, and accompanying drawings where:

FIG. 1 is a diagram of a blister pack holding dosage forms of inverse agonists or combinations of inverse agonists with other therapeutic agents for the treatment of chronic obstructive pulmonary disease or other diseases or conditions affecting the respiratory tract according to the present invention.

FIG. 2A is a graph showing that methacholine provocation significantly enhances airway resistance (Raw) in a mouse model of asthma.

FIG. 2B is a similar graph showing that saline provocation, as a control, does not significantly enhance airway resistance in a mouse model of asthma.

FIG. 2C is a similar graph showing that the administration of a single intravenous bolus of salbutamol to asthmatic mice reduced the level of airway responsiveness to methacholine provocation and the level of airway resistance.

FIG. 2D is a similar graph showing that no protection was observed when salbutamol was delivered to the mice for 28 days before methacholine provocation.

FIG. 2E is a similar graph showing that when asthmatic mice were given a single intravenous bolus of alprenolol, a β-adrenergic antagonist with partial agonist activity, their airway responsiveness was diminished.

FIG. 2F is a similar graph showing that when asthmatic mice were exposed to alprenolol for 28 days, their average methacholine dose-response relationship was similar to that obtained in nontreated mice, demonstrating that this drug provides no benefit upon chronic administration.

FIG. 2G is a similar graph showing that a single intravenous bolus of carvedilol enhanced the airway responsiveness in the mouse model of asthma.

FIG. 2H is a similar graph showing that chronic administration of carvedilol reduced the airway responsiveness of asthmatic mice to methacholine provocation.

FIG. 2I is a similar graph showing that a single intravenous bolus of nadolol also enhanced the airway responsiveness of asthmatic mice similar to the effect observed for carvedilol.

FIG. 2J is a similar graph showing that chronic administration of nadolol reduced the airway responsiveness of asthmatic mice to methacholine provocation similar to the effect observed for carvedilol on chronic administration of that drug.

FIG. 3 is a graph showing the effects of administration of β-adrenergic receptor ligands on the peak airway responsiveness to cholinergic stimulation: ((A), after treatment with the β-adrenergic agonist salbutamol; (B) after acute treatments with β-adrenergic receptor inverse agonists; and (C) after chronic treatments with β-adrenergic receptor inverse agonists).

FIG. 4 is a series of epifluorescent photomicrographs showing an increase in β-adrenergic receptor density upon treatment with nadolol.

FIG. 5A is graph showing the effect of combination therapy with carvedilol and salbutamol on airway hyperresponsiveness in asthmatic mice challenged with methacholine.

FIG. 5B is a summary graph showing the results presented in FIG. 5A.

FIG. 6 is a graph showing the effect of acute combination therapy with nadolol and aminophylline on airway hyperresponsiveness in asthmatic mice challenged with methacholine.

FIG. 7 is a graph showing the ratio of phospholipase C to actin in mice treated with various treatments, including long-term nadolol administration, to show that long-term nadolol administration decreases the activity of phospholipase C.

FIG. 8A is a graph showing the effects of salbutamol administration upon airway hyperresponsiveness.

FIG. 8B is a graph showing the effects of high-dose alprenolol administration upon airway hyperresponsiveness.

FIG. 8C is a graph showing the effects of low-dose alprenolol administration upon airway hyperresponsiveness.

FIG. 8D is a graph showing the effects of high-dose carvedilol administration upon airway hyperresponsiveness.

FIG. 8E is a graph showing the effects of low-dose carvedilol administration upon airway hyperresponsiveness.

FIG. 8F is a graph showing the effects of high-dose nadolol administration upon airway hyperresponsiveness.

FIG. 8G is a graph showing the effects of low-dose nadolol administration upon airway hyperresponsiveness.

FIG. 9 is a set of graphs showing the effects of long-term dosage of metoprolol and timolol upon airway hyperresponsiveness in asthmatic mice: (A) experimental results with metoprolol and timolol; (B) historical controls with non-challenged mice (Ctrl) and with challenged mice with no treatment (NTX).

FIG. 10 is a photomicrograph showing the occurrence of a mucus plug in the bronchus of an 8-year-old girl with fatal asthma.

FIG. 11 is a series of photomicrographs showing that nadolol is effective in preventing mucous metaplasia while the antagonist alprenolol is ineffective in preventing mucous metaplasia: top left, control; top right, sensitized/challenged mice without treatment showing mucous metaplasia; bottom left, sensitized/challenged mice after treatment with alprenolol showing no improvement in mucous metaplasia; bottom right, sensitized/challenged mice after treatment with nadolol showing nearly complete elimination of mucous metaplasia.

FIG. 12 is a schematic diagram showing the mechanism of action of nadolol as contrasted with the mechanism of action of long-acting β-adrenoceptor agonists (LABA) and that nadolol (“INV102”) reverses epithelial changes via inhibition of the β-arrestin pathway in β₂ airway receptors.

FIG. 13 is a graph showing the effect of nadolol on the level of mucin 5AC in smokers treated with nadolol versus the results with a placebo.

FIG. 14 is a graph showing the effect of nadolol on the success of smoking cessation (left panel) versus a placebo (right panel); administration of nadolol produced a 70% reduction in smoking in patients who had a history of failing to quit smoking programs at least 5 times.

FIG. 15 is a set of graphs showing that nadolol does not block the effectiveness of the administration of salbutamol (2.5 mg, administered by nebulization), administered after methacholine challenge in subjects with mild asthma.

FIG. 16 is a graph showing that nadolol blocks the β-arrestin pathway, as compared with carvedilol, propranolol, and alprenolol.

FIG. 17 is a set of photomicrographs showing the respiratory epithelium in: normal subject without airway disease (upper left); severe asthma (upper right); chronic bronchitis (lower left); and cystic fibrosis (lower right).

### DEFINITIONS

Although any methods and materials similar to or equivalent to those described herein can be used in the practice or testing of embodiments described herein or other embodiments within the scope of the invention, some preferred methods, compositions, materials, and devices are described herein. However, in this context, it must be understood that this invention is not limited to the particular molecules, compositions, methodologies, or protocols described herein, as these aspects of the invention may vary in accordance with routine experimentation and optimization as is generally known in the art. It is also to be understood that the terminology used in the description and the claims is for the purpose of describing the particular versions or embodiments only, and is not intended to limit the scope of the embodiments as described herein as understood by one of skill in the art.

Unless otherwise defined, all technical and scientific terms used herein have the same meanings as commonly understood by one of ordinary skill in the art to which this invention belongs. However, in case of any conflict of meanings, the present specification and claims, including definitions therein, shall control. Accordingly, in the context of the embodiments described herein, the following definitions apply.

As used herein and in the appended claims, the singular forms “a,” “an,” and “the” include references to the plural unless the context clearly dictates otherwise. Thus, for example, a reference to “an arrestin-2 inhibitor” is a reference to one or more arrestin-2 inhibitors or equivalents thereof known to those skilled in the art. As stated above, the terms “arrestin-2” and “β-arrestin” are used interchangeably herein.

As used herein, the terms “comprise,” “include,” and linguistic variations thereof denote the presence of recited features, elements, method steps, or other components of the invention without the exclusion of the presence of additional/recited features, elements, method steps, or other components. Conversely, the terms “consisting of” and linguistic variations thereof denote the presence of recited features, elements, method steps, or other components of the invention and exclude any unrecited recited features, elements, method steps, or other components of the invention except for ordinarily-associated impurities. The phrase “consisting essentially of” and linguistic variations thereof denote the presence of recited features, elements, method steps, or other components of the invention and any additional features, elements, method steps, or other components of the invention that do not materially affect the basic nature of the composition, system, or method. Many embodiments herein are described using open “comprising” language; such embodiments also encompass embodiments described in terms of “consisting essentially of” or “consisting of” language, which may be alternatively claimed or described using such language, unless the context clearly excludes “consisting essentially of” or “consisting of” language.

All chemical names used herein, including names of substituents, should be interpreted in light of the chemical nomenclature conventions of IUPAC and/or a modified format in which functional groups within a substituent are read in the order in which they branch from the scaffold or main structure. For example, in the modified nomenclature, methylsulfonylpropanol refers to CH₂SO₂CH₂CH₂CH₂OH or

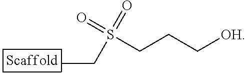

As another example, according to the modified nomenclature, a methylamine substituent is

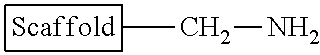

while an aminomethyl substituent is

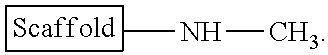

As used herein, the term “subject” broadly refers to any animal, including, but not limited to, humans and non-human mammals. The reference to non-human mammals includes, but is not limited to, socially or economically important animals or animals used for research including cattle, sheep, goats, horses, pigs, llamas, alpacas, dogs, cats, rabbits, guinea pigs, rats, and mice. Unless specified, methods and compositions according to the present invention are not limited to treatment of humans. In general, when treatment of humans is intended, the term “patient” can used in place of “subject.”

As used herein, the terms “effective amount,” “therapeutically effective amount,” or other equivalent terminology refer to the amount of a compound or compounds or to the amount of a composition sufficient to effect beneficial or desired results. The beneficial or desired results are typically a reduction in severity, symptoms, or duration of a disease or condition being treated and can generally be characterized as an amount of a therapeutic agent or composition effective to treat, ameliorate, or prevent a desired disease or condition, or to exhibit a detectable therapeutic or preventative effect. The use of such terminology cannot, unless specifically indicated, be interpreted as implying a complete cure for any disease or condition as recited herein. An effective amount can be administered in one or more administrations, applications, or dosages, and is not intended to be limited to a particular formulation or administration route unless a particular formulation or administration route is specified. The effect induced by the administration of a therapeutically effective amount can be detected by, for example, chemical markers, antigen levels, or changes in physiological indicators such as airway resistance. Therapeutic effects also include reduction in physical symptoms, such as decreased bronchoconstriction or decreased airway resistance, and can include subjective improvements in well-being, reduction of fatigue, or increased energy noted by the subjects or their caregivers. The precise therapeutically effective amount for a subject will depend upon the subject's size, weight, and health, the nature and extent of the condition affecting the subject, the administration of other therapeutics administered to treat the particular disease or condition being treated or other diseases or conditions affecting the subject, as well as variables such as liver and kidney function that affect the pharmacokinetics of administered therapeutics. Thus, it is not useful to specify an exact effective amount in advance. However, the therapeutically effective amount for a given situation can be determined by routine experimentation and is within the judgment of the clinician.

As used herein, the terms “administration,” “administering,” or other equivalent terminology, refer to the act of giving a drug, prodrug, pharmaceutical composition, or other agent intended to provide therapeutic treatment to a subject or in vivo, in vitro, or ex vivo to cells, tissues, or organs. Exemplary routes of administration to the human body can be through space under the arachnoid membrane of the brain or spinal cord (intrathecal), the eyes (ophthalmic), mouth (oral), skin (topical or transdermal), nose (nasal), lungs or other portions of the respiratory tract (inhalant), oral mucosa (buccal), ear, rectal, vaginal, by injection (such as, but not limited to, intravenously, subcutaneously, intraperitoneally, or by other injection routes as known in the art).

As used herein, the terms “co-administration” and “co-administering” refer to the administration of at least two agents, such as a β-adrenergic inverse agonist and an arrestin-2 inhibitor, or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful agent or agent, and/or when co-administration of two or more agents results in sensitization of a subject to beneficial effects of one of the agents via co-administration of the other agent. As used herein, the term “concurrent administration” refers to the administration of two or more active agents sufficiently close in time to achieve a combined therapeutic effect that is preferably greater than that which would be achieved by the administration of either agent alone. Such concurrent administration can be carried out simultaneously, e.g., by administering the active agents together in a common pharmaceutically acceptable carrier, thereby forming a pharmaceutical composition with two or more active agents, in one or more doses of the pharmaceutical composition.

As used herein, the term “pharmaceutical composition” refers to the combination of one or more active agents with at least one carrier, inert or active, making the composition especially suitable for diagnostic or therapeutic use in vitro, in vivo or ex vivo.

As used herein, the terms “pharmaceutically acceptable” or “pharmacologically acceptable,” as used herein, refer to compositions, or components within compositions, that do not substantially produce adverse reactions, such as, but not limited to, toxic, allergic, or unwanted immunological reactions, when administered to a subject.

As used herein, the term “pharmaceutically acceptable carrier” refers to any of the standard pharmaceutical carriers including, but not limited to, phosphate buffered saline solution, water, emulsions, such as oil/water or water/oil emulsions), and various types of wetting agents, any and all solvents, dispersion media, coatings, sodium lauryl sulfate, isotonic and absorption delaying agents, disintegrants such as potato starch or sodium starch glycolate), and the like. The carriers also can include stabilizers and preservatives.

As used herein, the term “pharmaceutically acceptable salt” refers to any pharmaceutically acceptable salt (e.g., acid or base) of a compound that is used in a method of the present invention or is a component of a composition of the present invention, which, upon administration to a subject, is capable of providing a compound of the present invention or an active metabolite or residue thereof. As is known to those of skill in the art, salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and other acids known in the art as suitable for formation of pharmaceutically acceptable salts. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metals (such as sodium or potassium) hydroxides, alkaline earth metals (such as calcium or magnesium), hydroxides, ammonia, and compounds of formula NW₄⁺, wherein W is C₁-C₄ alkyl, and the like. Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺, wherein W is a C₁-C₄ alkyl group), and the like. For therapeutic use, salts of the compounds herein are contemplated as being pharmaceutically acceptable. However, salts of acids and bases that are non-pharmaceutically acceptable may also find use, for example, in the preparation or purification of a pharmaceutically acceptable compound.

As used herein, the term “instructions for administering a compound to a subject,” and grammatical equivalents thereof, includes instructions for using the compositions contained in a kit for the treatment of conditions. Such instructions, for example, provide dosing, routes of administration, or decision trees for treating physicians for correlating patient-specific characteristics with therapeutic courses of action. Such instructions may be part of a kit according to the present invention.

As used herein, in the generally accepted two-state model of receptor theory, the term “agonist” is defined as a substance that has an affinity for the active site of a receptor and thereby preferentially stabilizes the active state of the receptor, or a substance, including, but not limited to, drugs, hormones, or neurotransmitters, that produces activation of receptors and enhances signaling by those receptors. Irrespective of the mechanism or mechanisms of action, an agonist produces activation of receptors and enhances signaling by those receptors.

As used herein, in the two-state model of receptor theory, the term “antagonist” is defined as a substance that does not preferentially stabilize either form of the receptor, active, or inactive, or a substance, including, but not limited to, drugs, hormones, and neurotransmitters, that prevents or hinders the effects of agonists and/or inverse agonists. Irrespective of the mechanism or mechanisms of action, an antagonist prevents or hinders the effects of agonists and/or inverse agonists.

As used herein, in the two-state model of receptor theory, the term “inverse agonist” is defined as a substance that has an affinity for the inactive state of a receptor and thereby preferentially stabilizes the inactive state of the receptor, or a substance, including, but not limited to, drugs, hormones, or neurotransmitters, that produces inactivation of receptors and/or prevents or hinders activation by agonists, thereby reducing signaling from those receptors.

As used herein, the term “arrestin-2 inhibitor” refers to any compound that directly or indirectly blocks one or more effects of arrestin-2 on β-adrenergic receptors, particularly β₂-adrenergic receptors and thus potentiates the activity of such receptors when bound to agonists. The compound can interact directly with arrestin-2 or can interact with one or more additional molecules that have the effect of stabilizing or activating arrestin-2.

As used herein, the term “alkyl” refers to an unbranched, branched, or cyclic saturated hydrocarbyl residue, or a combination thereof, of from 1 to 12 carbon atoms, or in some cases up to 50 or more carbon atoms, that can be optionally substituted; the alkyl residues contain only C and H when unsubstituted. Typically, the unbranched or branched saturated hydrocarbyl residue is from 1 to 6 carbon atoms, preferably 1 to 4 carbon atoms, more preferably 1 to 3 carbon atoms, which is referred to herein as “lower alkyl.” When the alkyl residue is cyclic and includes a ring, it is understood that the hydrocarbyl residue includes at least three carbon atoms, which is the minimum number to form a ring. An alkyl group can be linear, branched, cyclic, or a combination thereof, and may contain from 1 to 50 or more carbon atoms, such as a straight chain or branched C₁-C₂₀ alkane. Examples of alkyl groups include but are not limited to methyl, ethyl, propyl, isopropyl, cyclopropyl, butyl isomers (e.g. n-butyl, isobutyl, and tert-butyl), cyclobutyl isomers (e.g. cyclobutyl, methylcyclopropyl, etc.), pentyl isomers, cyclopentane isomers, hexyl isomers, cyclohexane isomers, and the like. Unless specified otherwise (e.g., substituted alkyl group, heteroalkyl, alkoxy group, haloalkyl, alkylamine, thioalkyl, or other groups containing at least one atom other than carbon or hydrogen), an alkyl group contains carbon and hydrogen atoms only. As used herein, the term “linear alkyl” refers to a chain of carbon and hydrogen atoms (e.g., ethane, propane, butane, pentane, hexane, or other examples). A linear alkyl group may be referred to by the designation —(CH₂)_{q}CH₃, where q is 0-49. The designation “C₁-C₁₂ alkyl” or a similar designation refers to alkyl having from 1 to 12 carbon atoms such as methyl, ethyl, propyl isomers (e.g. n-propyl or isopropyl), butyl isomers, cyclobutyl isomers (e.g. cyclobutyl or methylcyclopropyl), pentyl isomers, cyclopentyl isomers, hexyl isomers, cyclohexyl isomers, heptyl isomers, cycloheptyl isomers, octyl isomers, cyclooctyl isomers, nonyl isomers, cyclononyl isomers, decyl isomers, cyclodecyl isomers, or other alternatives known in the art. Similar designations refer to alkyl with a number of carbon atoms in a different range. As used herein, the term “Cₓ-C_{y}” when used in conjunction with a chemical moiety, such as alkyl, alkenyl, alkynyl, or carbocycle is meant to include groups that contain from x to y carbons in the chain or ring. For example, the term “Cₓ-C_{y} alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, or other alternatives. The terms “Cₓ-C_{y} alkenyl” and “Cₓ-C_{y} alkynyl” refer to substituted or unsubstituted unsaturated aliphatic groups analogous in length and possible substitution to the alkyls described above, but that contain at least one double or triple bond respectively. The term “Cₓ-C_{y} carbocycle” refers to a substituted or unsubstituted carbocycle, that contain from x to y ring carbons. As used herein, the term “branched alkyl” refers to a chain of carbon and hydrogen atoms, without double or triple bonds, that contains a fork, branch, and/or split in the chain (e.g., 3,5-dimethyl-2-ethylhexane, 2-methyl-pentane, 1-methyl-cyclobutane, ortho-diethyl-cyclohexane, or other alternatives). “Branching” refers to the divergence of a carbon chain, whereas “substitution” refers to the presence of non-carbon/non-hydrogen atoms in a moiety. Unless specified otherwise (e.g., substituted branched alkyl group, branched heteroalkyl, branched alkoxy group, branched haloalkyl, branched alkylamine, branched thioalkyl, or other alternatives), a branched alkyl group contains carbon and hydrogen atoms only.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising a heteroatom. The carbocycle can be fully saturated or partially saturated, but non-aromatic. For example, the general term “carbocyclyl” encompasses cycloalkyl. The carbocyclic and heterocyclic structures encompass compounds having monocyclic, bicyclic or multiple (polycyclic) ring systems; and such systems may mix aromatic, heterocyclic, and carbocyclic rings. Mixed ring systems are described according to the ring that is attached to the rest of the compound being described. Bicyclic or polycyclic rings may include fused or spiro rings. Carbocycles may include 3- to 10-membered monocyclic rings, 6- to 12-membered bicyclic rings, and 6- to 12-membered bridged rings. Each ring of a bicyclic or polycyclic carbocycle may be selected from saturated, unsaturated, and aromatic rings. In an exemplary embodiment, an aromatic carbocycle, e.g., phenyl, may be fused to a saturated or unsaturated ring, e.g., cyclohexane, cyclopentane, or cyclohexene. In some embodiments, the carbocycle is an aromatic carbocycle. In some embodiments, the carbocycle is a cycloalkyl. In some embodiments, the carbocycle is a cycloalkenyl. Exemplary carbocycles include cyclopentyl, cyclohexyl, cyclohexenyl, adamantyl, phenyl, indanyl, and naphthyl. An alkenyl group can be optionally substituted by one or more substituents such as those substituents described herein. A “non-aromatic carbocycle” includes rings and ring systems that are saturated, unsaturated, substituted or unsubstituted, but not aromatic or aryl rings or ring systems.

As used herein, the term “cycloalkyl” refers to a completely saturated mono- or multi-cyclic hydrocarbon ring system. When composed of two or more rings, the rings may be joined together in a fused, bridged or spiro-connected fashion. Cycloalkyl groups of the present application may range from three to ten carbons (C₃ to C₁₀). A cycloalkyl group may be unsubstituted, substituted, branched, and/or unbranched. Typical cycloalkyl groups include, but are not limited to, cyclopropyl, cyclobutyl, cyclopentyl, cyclohexyl, and the like. If substituted, the substituent(s) may be an alkyl or can be selected from those indicated above with regard to substitution of an alkyl group unless otherwise indicated. While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

As used herein, the term “heteroalkyl” refers to an alkyl group, as defined herein, wherein one or more carbon atoms are independently replaced by one or more heteroatoms (e.g., oxygen, sulfur, nitrogen, phosphorus, selenium, silicon, or combinations thereof). The alkyl group containing the non-carbon substitution(s) may be a linear alkyl, branched alkyl, cycloalkyl (e.g., cycloheteroalkyl), or combinations thereof. Non-carbons may be at terminal locations (e.g., 2-hexanol) or integral to an alkyl group (e.g., diethyl ether). In general, the “hetero” terms refer to groups that typically contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. In some cases, more than three heteroatoms may be present. Unless stated otherwise specifically in the specification, the heteroalkyl group may be optionally substituted as described herein.

Representative heteroalkyl groups include, but are not limited to —OCH₂OMe, —OCH₂CH₂OMe, or —OCH₂CH₂OCH₂CH₂NH₂. For reasons of chemical stability, it is also understood that, unless otherwise specified, such groups do not include more than two contiguous heteroatoms except where an oxo group is present on N or S as in a nitro or sulfonyl group.

As used herein, the term “heteroalkylene” refers to an alkyl radical as described above where one or more carbon atoms of the alkyl is replaced with a heteroatom, e.g., O, N or S, or another heteroatom as described above. “Heteroalkylene” or “heteroalkylene chain” refers to a straight or branched divalent heteroalkyl chain linking the rest of the molecule to a radical group. Unless stated otherwise specifically in the specification, the heteroalkylene group may be optionally substituted as described herein. Representative heteroalkylene groups include, but are not limited to —OCH₂CH₂O—, —OCH₂CH₂OCH₂CH₂O—, or —OCH₂CH₂OCH₂CH₂OCH₂CH₂O—.

As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule and such that a stable compound is formed thereby, i.e., a compound that does not spontaneously undergo transformation such as by rearrangement, cyclization, elimination, hydrolysis, lactone or lactam formation, or other reaction. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents. Substitution of a structure depicted herein may result in removal or moving of a double bond or other bond, as will be understood by one in the field. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds that do not significantly alter the pharmacological activity of the compound in the context of the present invention. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. The heteroatoms such as nitrogen may have hydrogen substituents and/or any permissible substituents of organic compounds described herein which satisfy the valences of the heteroatoms.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di-and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, or I). When an alkyl group is substituted with more than one halogen radical, each halogen may be independently selected e.g., 1-chloro, 2-fluoroethane.

As used herein, the term “aryl” refers to a monocyclic or fused bicyclic moiety having the well-known characteristics of aromaticity; examples include phenyl and naphthyl, which can be optionally substituted. Additional examples of aromatic rings include furan, benzofuran, isobenzofuran, pyrrole, indole, isoindole, thiophene, benzothiophene, benzo(c)thiophene, imidazole, benzimidazole, purine, pyrazole, indazole, oxazole, benzooxazole, isoxazole, benzisoxazole, thiazole, benzothiazole, benzene, naphthalene, pyridine, quinolone, isoquinoline, pyrazine, quinoxaline, pyrimidine, quinazoline, pyridazine, cinnoline, phthalazine, triazine (e.g., 1,2,3-triazine; 1,2,4-triazine; 1,3,5 triazine), and thiadiazole. The term “aromatic carbocycle” refers to an aromatic ring without heteroatoms present within the ring structure, such as, but not limited to benzene or naphthalene. Other terms that can be used include “aromatic ring,” “aryl group,” or “aryl ring.”

As used herein, the term “heterocycle,” “heterocyclyl,” “heterocyclic ring” or “heterocyclic group” is intended to mean a stable 4-, 5-, 6-, or 7-membered monocyclic or 7-, 8-, 9-, 10-, 11-, 12-, 13-, or 14-membered bicyclic heterocyclic ring which is saturated, partially unsaturated, or fully unsaturated or aromatic, and which consists of carbon atoms and 1, 2, 3 or 4 heteroatoms independently selected from N, O, and S; and including any bicyclic group in which any of the above-defined heterocyclic rings is fused to a benzene ring. Other heteroatoms, such as P, Se, B, or Si, can be included in some alternatives. The nitrogen and sulfur heteroatoms may optionally be oxidized. The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. When the term “heterocycle,” “heterocyclyl,” “heterocyclic ring” or “heterocyclic group” is used, it is intended to include heteroaryl unless heteroaryl is excluded. Examples of heterocycles include, but are not limited to, acridinyl, azocinyl, benzimidazolyl, benzofuranyl, benzothiofuranyl, benzoxazolyl, benzoxazolinyl, benzthiazolyl, benztriazolyl, benztetrazolyl, benzisoxazolyl, benzisothiazolyl, benzimidazolinyl, carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles.

As used herein, the term “non-aromatic heterocycle” refers to a cycloalkyl or cycloalkenyl, as defined herein, wherein one or more of the ring carbons are replaced by a moiety selected from —O—, —N═, —NR—, —C(O)—, —S—, —S(O)— or —S(O)₂—, wherein R is hydrogen, C₁-C₈ alkyl or a nitrogen protecting group, with the proviso that the ring of said group does not contain two adjacent O or S atoms. In some alternatives, other heteroatoms including P, Se, B, or Si can be included. Non-limiting examples of non-aromatic heterocycles, as used herein, include morpholino, pyrrolidinyl, pyrrolidinyl-2-one, piperazinyl, piperidinyl, piperidinylone, 1,4-dioxa-8-aza-spiro(4.5)dec-8-yl, 2H-pyrrolyl, 2-pyrrolinyl, 3-pyrrolinyl, 1,3-dioxolanyl, 2-imidazolinyl, imidazolidinyl, 2-pyrazolinyl, pyrazolidinyl, 1,4-dioxanyl, 1,4-dithianyl, thiomorpholinyl, azepanyl, hexahydro-1,4-diazepinyl, tetrahydrofuranyl, dihydrofuranyl, tetrahydrothienyl, tetrahydropyranyl, dihydropyranyl, tetrahydrothiopyranyl, thioxanyl, azetidinyl, oxetanyl, thietanyl, oxepanyl, thiepanyl, 1,2,3,6-tetrahydropyridinyl, 2H-pyranyl, 4H-pyranyl, dioxanyl, 1,3-dioxolanyl, dithianyl, dithiolanyl, dihydropyranyl, dihydrothienyl, dihydrofuranyl, imidazolinyl, imidazolidinyl, 3-azabicyclo(3.1.0)hexanyl, and 3-azabicyclo(4.1.0)heptanyl, 3,8-diazabicyclo(3.2.1)octanyl, and 2,5-diazabicyclo(2.2.1)heptanyl. In certain embodiments, a non-aromatic heterocyclic ring is aziridine, thiirane, oxirane, oxaziridine, dioxirane, azetidine, oxetan, thietane, diazetidine, dioxetane, dithietane, pyrrolidine, tetrahydrofuran, thiolane, imidazolidine, pyrazolidine, oxazolidine, isoxazolidine, thiazolidine, isothiazolidine, dioxolane, dithiolane, piperdine, oxane, thiane, piperazine, morpholine, thiomorpholine, dioxane, dithiane, trioxane, thithiane, azepane, oxepane, thiepane, homopiperazine, or azocane.

As used herein, the terms “heteroaryl” or “heteroaromatic” refer to monocyclic, bicyclic, or polycyclic ring systems, wherein at least one ring in the system is aromatic and contains at least one heteroatom, for example, nitrogen, oxygen and sulfur. Each ring of the heteroaromatic ring systems may contain 3 to 7 ring atoms. Exemplary heteroaromatic monocyclic ring systems include 5- to 7-membered rings whose ring structures include one to four heteroatoms, for example, one or two heteroatoms. The inclusion of a heteroatom permits aromaticity in 5-membered rings as well as in 6-membered rings. Typical heteroaromatic systems include monocyclic C₅-C₆ heteroaromatic groups such as pyridyl, pyrimidyl, pyrazinyl, thienyl, furanyl, pyrrolyl, pyrazolyl, thiazolyl, oxazolyl, triazolyl, triazinyl, tetrazolyl, tetrazinyl, and imidazolyl, as well as the fused bicyclic moieties formed by fusing one of these monocyclic heteroaromatic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolylpyridyl, quinazolinyl, quinoxalinyl, cinnolinyl, and other ring systems known in the art. Any monocyclic or fused ring bicyclic system that has the characteristics of aromaticity in terms of delocalized electron distribution throughout the ring system is included in this definition. This definition also includes bicyclic groups where at least the ring that is directly attached to the remainder of the molecule has the characteristics of aromaticity, including the delocalized electron distribution that is characteristic of aromaticity. Typically the ring systems contain 5 to 12 ring member atoms and up to four heteroatoms, wherein the heteroatoms are selected from the group consisting of N, O, and S. Frequently, the monocyclic heteroaryls contain 5 to 6 ring members and up to three heteroatoms selected from the group consisting of N, O, and S; frequently, the bicyclic heteroaryls contain 8 to 10 ring members and up to four heteroatoms selected from the group consisting of N, O, and S. The number and placement of heteroatoms in heteroaryl ring structures is in accordance with the well-known limitations of aromaticity and stability, where stability requires the heteroaromatic group to be stable enough to be exposed to water at physiological temperatures without rapid degradation. As used herein, the term “hydroxyheteroaryl” refers to a heteroaryl group including one or more hydroxyl groups as substituents; as further detailed below, further substituents can be optionally included. As used herein, the terms “haloaryl” and “haloheteroaryl” refer to aryl and heteroaryl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. As used herein, the terms “haloalkyl,” “haloalkenyl,” and “haloalkynyl” refer to alkyl, alkenyl, and alkynyl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. When a range of values is listed, such as for the number of carbon atoms in an alkyl group, it is intended to encompass each value and subrange within the range. For example, “C₁-C₆ alkyl” includes alkyl groups with 1, 2, 3, 4, 5, or 6 carbon atoms and all possible subranges.

As used herein, the term “hydroxyaryl” refers to an aryl group including one or more hydroxyl groups as substituents; as further detailed below, further substituents can be optionally included.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. The term “solvate” typically means a physical association of a compound involving varying degrees of ionic and/or covalent bonding, including hydrogen bonding. In certain instances the solvate will be capable of isolation, for example when one or more solvent atoms are incorporated into the crystal lattice of the crystalline solid. The term “solvate” encompasses both solution-phase and isolatable solvates. Suitable solvates in which the solvent is other than water include, but are not limited to, ethanolates or methanolates. When water is the solvent, the corresponding solvate is a “hydrate.” Examples of hydrates include, but are not limited to, hemihydrate, monohydrate, dihydrate, trihydrate, hexahydrate, and other hydrated forms. It should be understood by one of ordinary skill in the art that the pharmaceutically acceptable salt and/or prodrug of compounds described herein for use in methods or compositions according to the present invention may also exist in a solvate form. When the solvate is a hydrate, the hydrate is typically formed via hydration which is either part of the preparation of the present compound or through natural absorption of moisture by the anhydrous compound of the present invention. Additionally, compounds may exist as clathrates or other complexes, which are therapeutic agent-host inclusion complexes wherein the therapeutic agent and the host are present in stoichiometric or non-stoichiometric amounts.

As used herein, the term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolyzable esters of the present compounds are the compounds whose carboxyls are present in the form of hydrolyzable ester groups. That is, these esters are pharmaceutically acceptable and can be hydrolyzed to the corresponding carboxyl acid in vivo.

As used herein, the term “alkenyl” refers to an unbranched, branched or cyclic hydrocarbyl residue having one or more carbon-carbon double bonds. Typically, the hydrocarbyl residue has from 2 to 12 carbon atoms (C₂-C₁₂ alkenyl). In certain embodiments, an alkenyl comprises two to eight carbon atoms (C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. An alkenyl group can be optionally substituted by one or more substituents such as those substituents described herein. With respect to the use of “alkenyl,” the presence of multiple double bonds cannot product an aromatic ring structure.

As used herein, the term “alkynyl” refers to an unbranched, branched, or cyclic hydrocarbyl residue having one or more carbon-carbon triple bonds; the residue can also include one or more double bonds. Typically, the hydrocarbyl residue has from 2 to 12 carbon atoms (C₂-C₁₂ alkynyl). In certain embodiments, an alkenyl comprises two to eight carbon atoms (C₂-C₈ alkynyl). In certain embodiments, an alkenyl comprises two to six carbon atoms (i.e., C₂-C₆ alkynyl). In other embodiments, an alkenyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like. With respect to the use of “alkynyl,” the presence of multiple double bonds in addition to the one or more triple bonds cannot produce an aromatic ring structure.

As used herein, the term “alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₁₀ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises only one carbon atom (i.e., C₁ alkylene or a —CH₂-group.). An alkylene group can be optionally substituted by one or more substituents such as those substituents described herein.

As used herein, the term “alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). An alkenylene group can be optionally substituted by one or more substituents such as those substituents described herein.

As used herein “alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). An alkenylene group can be optionally substituted by one or more substituents such as those substituents described herein.

As used herein, the term “amine” or “amino” includes primary, secondary, and tertiary amines wherein each non-hydrogen group on nitrogen may be selected from alkyl, aryl, and the like. Amines include but are not limited to —NH₂, —NH-phenyl, —NH—CH₃, —NH—CH₂CH₃, and —N(CH₃)benzyl. The amino group can be optionally substituted. For example, the term can include NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “amide” or “amido” includes C- and N-amide groups, e.g., —C(O)NR₂, and —NRC(O)R groups, respectively, where R can be H, alkyl, aryl, or other groups, which can be optionally substituted. Amide groups therefore include but are not limited to —C(O)NH₂, —NHC(O)H, —C(O)NHCH₂CH₃, —NHC(O)CH₃,or —C(O)N(CH₂CH₃)phenyl.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

As used herein, similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S, more typically from N, O, and P. The term “heteroatom” can include, in some contexts, other atoms, including selenium, silicon, or boron.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent.

As used herein, the term “sulfamoyl” refers to a substituent with the structure —S(O₂)NH₂, wherein the nitrogen of the NH₂ portion of the group can be optionally substituted as described above.

As used herein, the term “carboxyl” refers to a group of the structure —C(O₂)H.

As used herein, the term “carbamyl” refers to a group of the structure —C(O₂)NH₂, wherein the nitrogen of the NH₂ portion of the group can be optionally substituted as described above.

As used herein, the terms “monoalkylaminoalkyl” and “dialkylaminoalkyl” refer to groups of the structure -Alk₁-NH-Alk₂ and -Alk₁-N(Alk₂)(Alk₃), wherein Alk₁, Alk₂, and Alk₃ refer to alkyl groups as described above.

As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂-Ar wherein Ar refers to an aryl group as described above. The term “aryloxyalkylsulfonyl” refers to a group of the structure —S(O)₂-Alk-O-Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above.

As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

As used herein, the term “absent” when used in reference to a functional group or substituent, particularly in reference to the chemical structure of a compound, means that the particular functional group or substituent is not present in the compound being described. When used in reference to a substituent, the absence of the substituent typically means that the bond to the substituent is absent and that absence of the bond is compensated for with a H atom. When used in reference to a position within a chain or ring, the absence of the position typically means that the two positions otherwise connected by the absent position are instead directly connected by a covalent bond.

### DETAILED DESCRIPTION OF THE INVENTION

The methods and compositions of the present invention are based on the ability of the β-adrenergic inverse agonist nadolol to inhibit the activity of the arrestin-2 (β-arrestin) pathway, particularly in blocking signaling at airway epithelial cell β₂ receptors.

### I. Properties of Nadolol

The structure of nadolol is shown below as Formula (I):

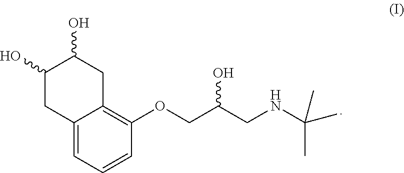 [SMILES: CC(C)(C)NCC(O)COc1cccc2c1CC(O)C(O)C2]

Nadolol is a mixture of four stereoisomers, shown below as Formulas (II(a)), (II(b)), (II(c)), and (II(d)):

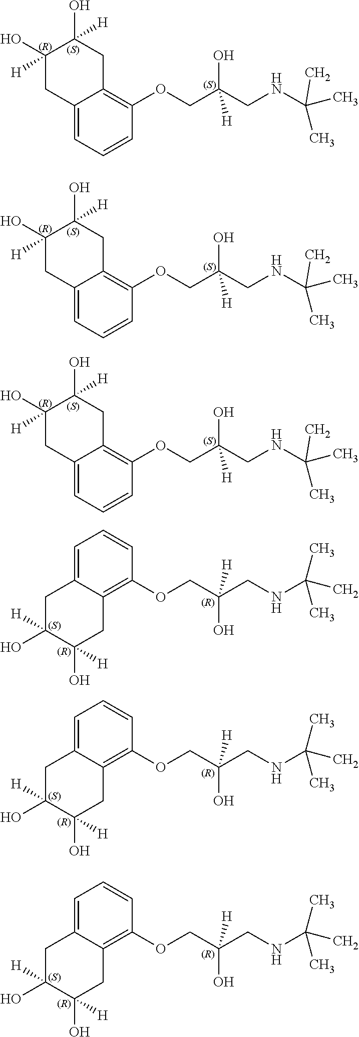

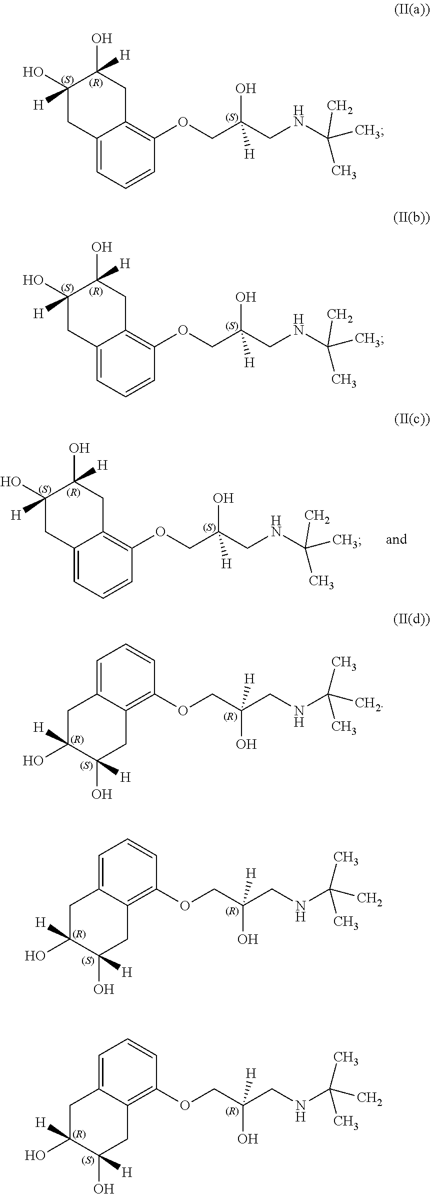

The most active stereoisomer of nadolol is the RSR stereoisomer.

Nadolol is polar and hydrophilic, with low lipid solubility.

As described below, in some alternatives, a derivative or analog of nadolol can be used. A particular derivative or analog of nadolol is a compound of Formula (I):

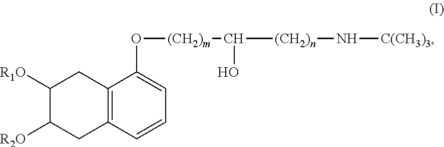

wherein R₁ is hydrogen or lower alkyl, R₂ is hydrogen or lower alkyl, and m and n are 1 to 3, with the proviso that wherein R₁ and R₂ are both hydrogen and m is 1, n is other than 1.

One embodiment of the invention is a method of treating a disease or condition affected by the modulation of the β-arrestin pathway by administering a therapeutically effective quantity of an inverse agonist for a β-adrenergic receptor, particularly a β₂-adrenergic receptor, whose modulation is involved in the disease or condition. Typically, the disease or condition is a respiratory disease or condition, including, but not limited to, asthma, chronic obstructive pulmonary disease (COPD), bronchitis, bronchiectasis, emphysema, allergic rhinitis, the pulmonary sequelae of cystic fibrosis, Churg-Strauss syndrome, pneumonia, and pulmonary symptoms associated with infection with SARS-CoV-2.

In classical receptor theory, two classes of G protein-coupled receptor (GPCR) ligands were considered: agonist and antagonist. Receptors were believed to exist in a single quiescent state that could only induce cellular signaling upon agonist binding to produce an activated receptor state. In this model, binding by antagonists produced no cellular signaling but simply prevented receptors from being bound and activated by agonists. Then, Costa and Herz demonstrated that receptors could be manipulated into a constitutive or spontaneously active state that produced cellular signaling in the absence of agonist occupation. They also provided evidence that certain compounds inactivate those spontaneously active receptors (T. Costa & A. Herz, “Antagonists with Negative Intrinsic Activity at 8 Opioid Receptors Coupled to GTP-Binding Proteins,” Proc. Natl. Acad. Sci. USA 86: 7321-7325 (1989)). There is further evidence that GPCRs exist in constitutively or spontaneously active states that are inactivated to some degree by inverse agonists (R. A. de Ligt et al., “Inverse Agonism at G Protein-Coupled Receptors: (Patho)physiological Relevance and Implications for Drug Discovery,” Br. J. Pharmacol. 130: 1-12 (2000); G. Milligan et al., “Inverse Agonism: Pharmacological Curiosity or Potential Therapeutic Strategy?,” Trends Pharmacol. Sci. 16: 10-13 (2000)).

The basis of the use of inverse agonists such as nadolol to modulate the activity of β-adrenergic receptors, particularly β₂-adrenergic receptors, in general, involves the recognition of the existence of inverse agonists and the understanding of the effect that chronic treatment with inverse agonists has on receptor function. What is an inverse agonist and how does it function? Receptors, such as β-adrenergic receptors (“β-adrenoceptors”) that respond to adrenalin (epinephrine), typically exist in an equilibrium between two states, an active state and an inactive state. When receptors bind to agonists, such as adrenalin for the β-adrenoceptor, they stop them from cycling back into the inactive state, thus shifting the equilibrium between the active and inactive states according to the Law of Mass Action. This occurs because those receptors bound to agonists are removed from the equilibrium. Typically, antagonists bind to the receptors, but prevent the binding of agonists. However, molecules known as “inverse agonists” bind to the receptors in the inactive state, causing the equilibrium between the active and the inactive state to shift toward the inactive state. This is not merely a matter of blocking agonist binding. Moreover, there is a population of spontaneously active receptors in vivo. These receptors provide a baseline constitutive level of activity; the activity is never entirely “off.”

As indicated above, it has been well demonstrated that chronic administration of β-adrenergic agonists causes agonist-dependent desensitization. Upon acute administration of β-agonists, adrenergic receptors are internalized, thereby preventing them from being restimulated further for pulmonary relaxation. With chronic administration of β-agonists, there is actually a downregulation in the total number of β-adrenergic receptors. The consequence may be the observed loss of responsiveness seen in asthmatic patients or in other patients with chronic respiratory disease on long-acting β-agonists, and referred to as tolerance or tachyphylaxis, as described above.

One aspect of treatment methods according to the present invention is based on the discovery that a chronic administration of an inverse agonist has the effect of upregulating the population of active β-adrenoceptors. The observed activity may be due to the receptor's constitutive baseline activity or the combined effect of increased level of receptors responding to endogenous agonists. This leads to the seemingly paradoxical result that the administration of a drug that would appear, at first blush, to degrade a physiological function, such as by causing airway hyperresponsiveness in asthma, can, if administered chronically, enhance that physiological function by upregulating the population of spontaneously active β-adrenergic receptors associated with that physiological function. This is a specific application of the principle of “paradoxical pharmacology” (R. Lin et al., “Changes in β₂-Adrenoceptor and Other Signaling Proteins Produced by Chronic Administration of “Beta-Blockers” in a Murine Asthma Model,” Pulm. Pharmacol. Ther. 21: 115-124 (2008)), and is particularly applicable to moderate to severe asthma as well as to other respiratory diseases or conditions.

Along these lines, the use of cardioselective β-adrenergic inverse agonists (those with a preference for the β₁-adrenergic receptor subtype) has been demonstrated to be safe in hypertensive and congestive heart failure (CHF) patients with chronic obstructive pulmonary disease (COPD).

As stated below, the present invention provides for the use of the active β-adrenoceptor receptor binding forms of β-adrenergic inverse agonists in the treatment of COPD and other diseases that are marked by airway hyperresponsiveness, including, but not limited to, emphysema, asthma, Churg-Strauss syndrome, chronic bronchitis, and bronchiectasis. A particularly preferred β-adrenergic inverse agonist is nadolol, which, as stated below, has the additional property of inhibiting arrestin-2 (β-arrestin). The inverse agonists can be in pure or substantially pure enantiomeric or diastereomeric form or can be racemic mixtures. In many cases, the active form of such compounds is the L form when the compound has only one chiral center. In the case of nadolol, which has three chiral centers and potentially 12 isomers, though, typically, only two are formed during synthesis, the most active form is the RSR form of nadolol (the designation of chiral centers in nadolol is according to the Cahn-Ingold-Prelog system). As stated above, in some alternatives, a derivative or analog of nadolol such as, but not limited to, the compound of Formula (I) can be used. As used herein, “analog” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or hydrophobic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring variant of the original compound. Other types of analogues include isomers (enantiomers, diastereomers, and the like) and other types of chiral variants of a compound, as well as structural isomers. As used herein, “derivative” refers to a chemically or biologically modified version of a chemical compound that is structurally similar to a parent compound and (actually or theoretically) derivable from that parent compound. A “derivative” differs from an “analog” in that a parent compound may be the starting material to generate a “derivative,” whereas the parent compound may not necessarily be used as the starting material to generate an “analog.” A derivative may or may not have different chemical or physical properties than the parent compound. For example, the derivative may be more hydrophilic or hydrophobic or it may have altered reactivity as compared to the parent compound. Derivatization (i.e., modification) may involve substitution of one or more moieties within the molecule (e.g., a change in functional group). The term “derivative” also includes conjugates and prodrugs of a parent compound (i.e., chemically modified derivatives which can be converted into the original compound under physiological conditions).

Although a number of inverse agonists having activity at β-adrenergic receptors in tissues of the respiratory tract are known, including: nadolol, typically used as the hydrochloride; bupranolol, typically used as the hydrochloride; carazolol, typically used as the hydrochloride; carvedilol, typically used as the hydrochloride; ICI-118,551 (3-(isopropylamino)-1-[(7-methyl-4-indanyl)oxy]butan-2-ol), typically used as the hydrochloride; levobunolol, typically used as the hydrochloride; metoprolol, typically used as the tartrate or succinate; sotalol, typically used as the hydrochloride; and timolol, typically used as the hydrochloride, as well as solvates of these compounds and other salts of these compounds and prodrugs of these compounds, the properties of nadolol make it the preferred compound to be used in methods and compositions of the present invention. The properties of nadolol that make it the preferred compound to be used in methods and compositions according to the present invention include the inhibition of arrestin 2 (β-arrestin) as discussed below.

### II. Arrestin-2 (β-Arrestin)

Arrestins, which include arrestin-2 (β-arrestin), and other members of the family, including arrestin-1, arrestin-3, and arrestin-4, are members of a small family of proteins important for regulating signal transduction at G-protein-coupled receptors (GPCRs). In response to a stimulus, GPCRs activate heterotrimeric G proteins. In order to turn off this response, or to adapt to a persistent stimulus, active receptors need to be sensitized. The first step in desensitization is phosphorylation of G-protein-coupled receptors by a series of serine/threonine kinases called G protein coupled receptor kinases (GRKs). GRK-mediated phosphorylation specifically prepares the activated receptor for arrestin binding. Arrestin binding to the receptor then blocks further G-protein-mediated signaling and targets receptors for internalization, and redirects signaling to alternative G-protein-independent pathways, such as β-arrestin-mediated signaling. According to J. S. Smith et al., “Biased Signaling: From Simple Switches to Allosteric Microprocessors,” Nat. Rev. Drug Discovery 17: 243-260 (2018), G protein-coupled receptors (GPCRs) are the most common receptors encoded in the genome, comprising greater than 1% of the coding human genome with approximately 800 members and expressed within every organ system. All GPCRs share a common architecture consisting of an extracellular N-terminal sequence, seven transmembrane-spanning (TM) domains (TM1-TM7) that are connected by three extracellular and three intracellular loops, and an intracellular C-terminal domain. GPCRs are sensors of a wide array of extracellular stimuli, including proteins, hormones, small molecules, neurotransmitters, ions, and light. GPCR signaling is primarily controlled by interactions with three protein families: G proteins, G protein receptor kinases (GRKs) and β-arrestins (arrestin-2 proteins) which perform distinct functions at the receptor. Upon stimulation, GPCRs activate heterotrimeric G proteins. Classically, agonist binding causes a conformational change in a GPCR, inducing guanine exchange factor (GEF) activity that catalyzes the exchange of GTP for GDP on Ga subunits of the heterotrimeric G-protein. This, in turn, leads to the dissociation of the heterotrimeric complex into Gα and Gβγ subunits. The dissociated subunits promote the formation of second messenger effectors such as cyclic adenosine monophosphate (cAMP), inositol triphosphate (IP3), diacylglycerol (DAG), and other second messengers, as well as modulation of other receptors and channels, such as activation of inward-rectifying potassium channels.

Similar to most biological systems, negative feedback loops have evolved to quench sustained second messenger signaling following receptor stimulation for maintenance of biological homeostasis. After ligand binding and G protein activation, the receptor is phosphorylated on its cytoplasmic loops and C-terminus, primarily by GRKs, which enhance arrestin-2 binding to the receptor. Arrestins, including arrestin-2, were first discovered for their role in mediating receptor desensitization, the process whereby repeated stimulation decreases the signaling response over seconds to minutes, through steric hindrance of GPCR interaction with the G proteins. Arrestin-2 also mediates receptor internalization via interactions with clathrin coated pits. This can result in downregulation, a sustained decrease in receptor number over minutes to hours due to trafficking of these internalized receptors to proteasomes or lysosomes. Internalized receptors that are not degraded also can be recycled to the plasma membrane. It is now established that in addition to acting as negative regulators of G protein signaling, arrestin-2 also couples to numerous signaling mediators including mitogen-activated protein kinases (MAPKs), AKT, SRC, nuclear factor-κB (NF-κB) and phosphoinositide 3-kinase (PI3K) by acting as adaptors and scaffolds. These pathways are separate from classical G protein signaling, but can involve similar signaling cascades that are often temporally distinct. More recently, it has also been appreciated that some receptors tightly interacting with arrestin-2 maintain catalytic GEF (guanine nucleotide exchange factor) activity on endosomes, continuing to promote G protein signaling after internalization. Thus, arrestin-2 can regulate nearly all aspects of receptor activity, including desensitization, downregulation, trafficking and signaling.

Most drugs that activate or block GPCRs are thought to equally, or substantially equally, target distinct signaling pathways mediated by different G proteins and β-arrestins. These agonists are thought to amplify downstream signaling pathways in a similar fashion to that of the endogenous reference agonist, which are frequently referred to as balanced agonists, while most antagonists are believed to inhibit all second-messenger systems activated by those agonists. However, it was appreciated previously that selective agonists or antagonists could specifically target a particular receptor-linked effector system or a limited number of receptor-linked effector systems. Indeed, a number of ligands have been described that selectively activate some pathways while blocking others downstream of a receptor. Compared with the balanced agonists described above, these “functionally selective” or “biased” agonists can selectively activate G proteins while blocking arrestins, or vice versa. This behavior was initially identified in a number of GPCR systems, including pituitary adenylate cyclase-activating peptide (PACAP) receptor ligands that differentially activated different G proteins as measured by a reversal in potencies. Biased agonism has become an increasingly active area of research since the discovery of β-arrestin-mediated signaling, with a plethora of biased ligands identified for multiple GPCRs.

The discovery of biased agonism has had important implications for understanding of GPCR biology. First, biased agonism is not consistent with two-state models for receptor signaling, and therefore it alters the conventionally understood concept of efficacy. As used herein, the term “efficacy” is defined as the ability of a ligand to generate a quantifiable response after binding to a receptor. The quantifiable response is typically, but not necessarily, a response that promotes a normal physiological function or a treatment for a disease or condition.

Second, biased signaling suggests that GPCRs should not be modelled as binary switches but instead should be modelled as allosteric microprocessors that generate a multitude of conformations in response to different ligands. There are also important clinical implications for these ligands, as selectively activating or inhibiting specific signaling cascades could yield more targeted drugs with reduced side effects.

The factors that result in the development of a biased signaling response are as follows. Using the ternary complex as a general model for receptor activity, agonist activation of a receptor requires three principal components for initiating signaling: a ligand, a receptor, and at least one transducer or transducers (the number of transducers depends on the particular system). These three (or more in some cases) components act allosterically; for example, a ligand can increase the affinity of a receptor for a transducer with which it interacts, such as a G protein or β-arrestin, while the binding of the transducer to an intracellular receptor domain can stabilize a conformation that increases the affinity for a specific ligand. Allostery is a widespread phenomenon that describes the ability of interactions occurring at a site of a macromolecule to modulate interactions at a spatially distinct binding site on the same macromolecule in a reciprocal manner. The concept of allostery was first developed with regard to enzymes, but is also applicable to many other proteins, particularly, but not exclusively, multi-subunit proteins. As used herein “affinity,” is a measurement of how well a ligand binds to a receptor, commonly expressed in terms of a dissociation constant (K_{d}). The smaller the dissociation constant (expressed in molar units), the more tightly bound the ligand is to the receptor, and thus the higher the affinity between the ligand and the receptor. Affinity depends on cellular context, and therefore, affinity for a G protein-coupled receptor, such as the β₂-adrenergic receptor, is influenced by transducers, such as G proteins or β-arrestin, also known as arrestin-2.

In two-state models of ligand-receptor interaction and signaling, there are only binary conditions for the receptor: the inactive state, which is incapable of signaling, and the active state, which can bind to and activate transducers. The receptor is modelled as a switch, with agonists stabilizing the “on” state and antagonists stabilizing the “off” state. Agonist efficacy can be defined as the ability of a ligand to modify the signaling state of the receptor by stabilizing the active receptor conformation. The phenomenon of biased agonism demonstrates that receptors are not acting as simple switches that merely encode states of activity across a binary spectrum, that is, either agonists or antagonists that equally activate or inhibit all signaling pathways downstream of a receptor. Rather, ligand binding results in the activation or inhibition of multiple GPCR-mediated effectors, including, in this context, β-adrenergic receptors such as the β₂ receptor. These effectors often rely on distinct phases of G protein, GRK and β-arrestin signaling. Instead of encoding binary “on” or “off” signals, a more appropriate model is one where a GPCR acts as an allosteric microprocessor with pluridimensional efficacy, responding to different molecules with different transducer coupling efficiencies. Any site on the receptor surface that binds to a molecule can, in theory, stabilize a distinct receptor conformation and induce a particular pharmacological output. Therefore, the physiological activity of a drug need not necessarily be linked to an interaction at the orthosteric binding site. Any of the three components of a ternary complex as described above, the ligand, the receptor, and the transducer or transducers, can contribute to such a biased response. The general understanding of ligand bias is that it is transmitted through the receptor to downstream transducers as a result of ligand-induced generation of distinct conformations of an allosteric receptor. This may involve participation of a number of mechanisms, including changes in the secondary or tertiary structure of the receptor and the recruitment of proteins that post-translationally modify the intracellular loops and the C-terminal of the receptor, such as by phosphorylation or ubiquitylation.

Arrestin-2 blocks GPCR coupling in two ways. First, arrestin-2 binding to the cytoplasmic face of the receptor occludes the binding site for heterotrimeric G-protein, thus preventing its activation and resulting in desensitization. Secondly, arrestin-2 links the receptor to elements of the internalization machinery, clathrin (which is a protein that builds small vesicles in order to transport molecules within cells) and clathrin adaptor AP2, which promotes receptor internalization via coated pits and subsequent transport to internal cellular compartments, called endosomes. Subsequently, the receptor can either be directed to degradation compartments (lysosomes) or recycled back to the plasma membrane where it can again participate in signaling. The strength of arrestin-receptor interactions plays a role in which of these alternatives occur: tighter complexes tend to increase the probability of receptor degradation (Class B), while more transient complexes favor recycling (Class A), although the strength of the interaction is not completely determinative.

Arrestins, including arrestin-2, are elongated molecules in which several intramolecular interactions determine the relative origins of the two domains of the protein. In an unstimulated cell, arrestins are localized in the cytoplasm in a basal, “inactive” conformation. Active phosphorylated GPCRs recruit arrestin to the plasma membrane. Receptor binding induces a global conformational change that involves the movement of the two arrestin domains and the release of its C-terminal tail that contains clathrin and AP2 binding sites. Increased accessibility of these sites in receptor-bound arrestin targets the arrestin-receptor complex to the coated pit as described above. Arrestins also bind microtubules (part of the cellular “skeleton”), where they assume yet another conformation, different from both free and receptor-bound form. Microtubule-bound arrestins recruit certain proteins to the cytoskeleton, which affects their activity and/or redirects it to microtubule-associated proteins. Arrestins shuttle between the cell nucleus and the cytoplasm. Their nuclear functions are not fully understood, but it has been shown that all four mammalian arrestin subtypes remove some of their partners, such as protein kinase JNK3 or the ubiquitin ligase Mdm2, from the nucleus. Arrestins also modify gene expression by enhancing transcription of certain genes.

Desensitization of GPCRs, a phenomenon described above that is responsible for the loss of activity of agents such as β-adrenergic agonists typically occurring with chronic administration of such agents, has several mechanisms. Briefly, the classical paradigm for desensitization involves the dual step of receptor phosphorylation by second messenger-stimulated protein kinases (i.e., PKA or PKC, termed heterologous desensitization) or specific G-protein-coupled receptor kinases (GRKs, termed homologous desensitization) and subsequent binding of arrestin to sterically interdict further coupling between the receptor and the G protein. Together, these two actions regulate functional coupling of receptors to effector molecules and control the subcellular localization of the receptor during the process of agonist-induced homologous desensitization. However, another arrestin-2-mediated regulatory mechanism for desensitization involves the degradation of a second messenger, such as adenylyl cyclase-generated cAMP, by scaffolding phosphodiesterases (PDEs) to the vicinity of the effector. Whereas these two desensitization processes were thought in the past to be independent of one another, recruitment of both arrestin-2 and PDE to activated β-adrenoceptors showed very similar kinetics, suggesting that arestin-2 may serve as shuttles for the translocation of PDE to the activated receptor. This observation was strengthened by the fact that cells lacking arrestin-2 expression were unable to recruit PDE following β-adrenoceptor stimulation, a function that was rescued by the introduction of exogenous arrestin-2 to cells. Arrestin-2-mediated recruitment of PDE to activated β-adrenoceptors was shown in a subsequent study performed in cardiac myocytes to promote the switching from Gs to Gi coupling, thus shifting the receptor toward a pathway that further limits cAMP production, thus blocking further signal transmission by the β-adrenoceptors.

The exposure of agonist results in trafficking of GPCRs into intracellular compartments in a process of sequestration or internalization. Internalization was initially identified as a critical step in resensitization of desensitized receptors but more recently was shown to initiate cellular signaling such as mitogenic pathways. A central role for arrestin-2 in the internalization process was discovered by showing that overexpression of arrestin-2 rescues internalization-deficient β-adrenoceptor mutants impaired in their sequestration ability and conversely that β-adrenoceptor internalization can be inhibited with arrestin-2-defective mutants.

The mechanism by which arrestin-2 mediates receptor internalization is via its ability to interact with proteins of the clathrin-coated pit (CCP) machinery. It is now understood that arrestin-2 functions as an adaptor molecule that binds directly to clathrin via the adaptor protein AP-2. AP-2 recruitment to the plasma membrane is facilitated by a direct agonist-dependent interaction between GRK2 and PI3 kinase in the cytosol, followed by rapid translocation of both enzymes to the plasma membrane where they interact with agonist-activated receptors. The generation of 3,4,5-phosphatidylinositols at the membrane by PI3 kinase enhances the recruitment of AP-2, thus promoting endocytosis. Interestingly, the internalization process also requires protein kinase activity of PI3 kinase, which acts to phosphorylate cytoskeletal tropomyosin, allowing for actin polymerization.

Complex formation between the receptor and the components of the sequestration machinery depends on the phosphorylation state of arrestin-2. In the unstimulated state, arrestin-2 is constitutively phosphorylated by the mitogen-activated protein kinase (MAPK) ERK1/2, thus reducing its capacity to interact with clathrin and thus reducing sequestration of GPCRs. On agonist stimulation of GPCRs such as β-adrenoceptors, cytoplasmic arrestin-2 is recruited to the plasma membrane, where it is rapidly dephosphorylated, an action that promotes its binding to actin and subsequent endocytosis.

Once internalized into intracellular compartments, receptors, such as β-adrenoceptors, are destined to either recycle back to the plasma membrane or, alternatively, become targeted for postendocytic degradation. One of the cellular processes that determines the fate of the receptor is ubiquitination—the adding of multiple ubiquitin molecules to lysine residues of the substrate protein, an action that marks it for degradation by the proteasome. Indeed, several GPCRs, including β-adrenoceptors, undergo agonist-mediated ubiquitination. However, although ubiquitination of the 2-adrenoceptor is necessary for receptor degradation, concomitantly occurring ubiquitination of arrestin-2 on agonist stimulation is also required for β₂-adrenoceptor internalization. Results with mutants suggest that the ubiquitination status of arrestin-2 determines the stability of the receptor-arrestin-2 complex in addition to its trafficking pattern.

Arrestin-2 also functions as an adaptor molecule that promotes the formation of multi-protein signaling complexes with proteins such as ERK and receptor and nonreceptor tyrosine kinases. This interaction is G-protein independent and functions to activate mitogenic pathways, such as those mediated by the ERK1/2-MAPK cascade.

The interaction of arrestin-2 with an activated receptor is mediated by the members of the family of GRKs; for the β₂-adrenoceptors, these members include at least GRK5 and GRK6. These GRKs induce receptor phosphorylation that leads to agonist-stimulated ERK activation in the absence of G-protein activation for β₂-adrenoceptors.

In addition, the CXC4 receptor has a role in activating inflammation through activation of arrestin-2-mediated pathways.

These different roles and activities of arrestin-2 are further addressed below in terms of therapeutic methods and compositions, including therapeutic methods and compositions involving proteins with which arrestin-2 has been shown to interact.

### III. Methods of Treatment of COPD and Other Respiratory Conditions

Accordingly, one aspect of the present invention is a method for treating chronic obstructive pulmonary disease (COPD) and other respiratory diseases or conditions as described above by administration of a therapeutically effective quantity of nadolol or a derivative or analog thereof to a patient with COPD to inhibit the activity of arrestin-2, thereby preventing the desensitization of β₂-adrenoceptors.

Preferably, the nadolol or derivative or analog of nadolol is nadolol itself. Alternatively, also expected to be within the scope of the invention are analogs of nadolol of Formula (I):

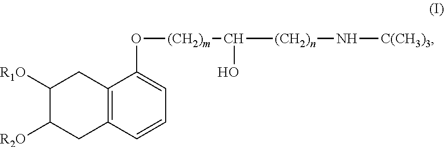

wherein R₁ is hydrogen or lower alkyl, R₂ is hydrogen or lower alkyl, and m and n are 1 to 3, with the proviso that where R₁ and R₂ are both hydrogen and m is 1, n is other than 1. As used herein, the term “lower alkyl” is defined as a straight or branched hydrocarbyl residue of 1 to 6 carbon atoms, more preferably 1 to 3 carbon atoms. Other derivatives or analogs of nadolol are described below.

The derivatives or analogs of nadolol that can be used in methods according to the present invention can include salts where a moiety present in the derivative or analog of nadolol has one or more groups that can either accept or donate protons, depending on the pH of the solution in which they are present. These moieties include carboxyl moieties, hydroxyl moieties, amino moieties, sulfonic acid moieties, and other moieties known to be involved in acid-base reactions. The recitation of a derivative or analog of nadolol include such salt forms as occur at physiological pH or at the pH of a pharmaceutical composition unless specifically excluded. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, p-hydroxybutyrates, glycolates, tartrates, methane-sulfonates, propanesulfonates, naphthalene-1-sulfonates, naphthalene-2-sulfonates, and mandelates. If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

Similarly, prodrug esters can be formed by reaction of either a carboxyl, a hydroxyl, or a sulfonic acid moiety on compounds or analogs or derivatives thereof suitable for methods according to the present invention with either an acid (for hydroxyl moieties) or an alcohol (for carboxyl or sulfonic acid moieties) to form an ester. Typically, the acid or alcohol includes a lower alkyl group of 1 to 6 carbons, such as methyl, ethyl, propyl, isopropyl, butyl, isobutyl, and tertiary butyl. These groups can be further substituted with substituents such as hydroxy or other substituents as described above as long as such substituents would not substantially impair the hydrolysis of the prodrug or the bioavailability of the resulting hydrolysis product. Such prodrugs are well known in the art and need not be described further here. The prodrug is converted into the active compound by hydrolysis of the ester linkage, typically by intracellular enzymes. Other suitable moieties that can be used to form prodrug esters are well known in the art. For example, prodrugs can include amides prepared by reaction of the parent acid compound with a suitable amine. In some cases it is desirable to prepare double ester type prodrugs such as (acyloxy) alkyl esters or ((alkoxycarbonyl)oxy)alkyl esters. Suitable esters as prodrugs include, but are not necessarily limited to, methyl, ethyl, propyl, isopropyl, n-butyl, isobutyl, t-butyl, morpholinoethyl, and N,N-diethylglycolamido. Methyl ester prodrugs may be prepared by reaction of the acid form of a compound having a suitable carboxylic acid group in a medium such as methanol with an acid or base esterification catalyst (e.g., NaOH or H2SO₄). Ethyl ester prodrugs are prepared in similar fashion using ethanol in place of methanol. Morpholinylethyl ester prodrugs may be prepared by reaction of the sodium salt of a suitable compound (in a medium such as dimethylformamide) with 4-(2-chloroethyl)morphine hydrochloride (available from Aldrich Chemical Co., Milwaukee, Wis. USA). The use of prodrug systems is described in T. Järvinen et al., “Design and Pharmaceutical Applications of Prodrugs” in Drug Discovery Handbook (S. C. Gad, ed., Wiley-Interscience, Hoboken, NJ, 2005), ch. 17, pp. 733-796.

Pharmaceutically acceptable salts include salts as described above. Other pharmaceutically acceptable salts are known in the art.

The subject to be treated can be a human patient or a socially or economically important animal as described above. Methods and compositions according to the present invention are not limited to the treatment of humans unless so specified.

Typically, when sustained-release oral administration of the nadolol, the derivative or analog of nadolol, or the prodrug of the nadolol or of the derivative or analog of nadolol is used, the administration results in continuous levels of the nadolol, the derivative or analog of nadolol, or, in the case of administration of a prodrug, an active agent resulting from the in vivo metabolism of the prodrug in the bloodstream of the subject. Typically, the method exerts a therapeutic effect that is an upregulation of pulmonary β₂-adrenoceptors. Typically, the method also exerts a therapeutic effect that is an inhibition of the activity of arrestin-2 at pulmonary β₂-adrenoceptors. Typically, the method also exerts a therapeutic effect that is increased pulmonary relaxation responsiveness to 2-adrenergic agonist drugs. This provides for combination therapy, described in detail below. Typically, the nadolol is administered by inhalation, as described in detail below.

The nadolol, the derivative or analog of nadolol, or the prodrug of the nadolol or the derivative or analog of nadolol can be administered in conjunction with one or more pharmaceutical excipients. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. The nadolol, the derivative or analog of nadolol, or the prodrug of the nadolol or the derivative or analog of nadolol can be administered in conjunction with one or more pharmaceutically acceptable carriers. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. Other pharmaceutical excipients and carriers are known in the art, and include, but are not limited to: preservatives; sweetening agents for oral administration; thickening agents; buffers; liquid carriers; wetting, solubilizing, or emulsifying agents; acidifying agents; antioxidants; alkalinizing agents; carrying agents; chelating agents; colorants; complexing agents; suspending or viscosity-increasing agents; flavors or perfumes; oils; penetration enhancers; polymers; stiffening agents; proteins; carbohydrates; bulking agents; and lubricating agents. The use of such agents for pharmaceutically active substances is well known in the art, and suitable agents for inclusion into dosage forms can be chosen according to factors such as the quantity of nadolol or other active agent to be included per unit dose, the intended route of administration, the physical form of the dosage form, and optimization of patient compliance with administration. Except insofar as any conventional medium, carrier, or agent is incompatible with the active ingredient or ingredients, its use in a composition according to the present invention is contemplated. Supplementary active ingredients can also be incorporated into the compositions, especially as described below under combination therapy. For administration of any of the compounds used in the present invention, preparations should meet sterility, pyrogenicity, general safety, and purity standards as required by the FDA Office of Biologics Standards or by other regulatory organizations regulating drugs.

Thus, the nadolol, the derivative or analog of nadolol, or the prodrug of the nadolol or the derivative or analog of the nadolol can be formulated for oral, sustained-release oral, buccal, sublingual, inhalation, insufflation, or parenteral administration.

If the nadolol, the derivative or analog of nadolol, or the prodrug of the nadolol or the derivative or analog of the nadolol is administered orally, either in a conventional or a sustained-release preparation, it is typically administered in a conventional unit dosage form such as a tablet, a capsule, a pill, a troche, a wafer, a powder, or a liquid such as a solution, a suspension, a tincture, or a syrup. Oral formulations typically include such normally employed excipients as, for example, pharmaceutical grades of mannitol, lactose, starch, magnesium stearate, sodium saccharin, cellulose, magnesium carbonate, and other conventional pharmaceutical excipients. In certain defined embodiments, oral pharmaceutical compositions will comprise an inert diluent and/or assimilable edible carrier, and/or they may be enclosed in hard or soft shell gelatin capsules. Alternatively, they may be compressed into tablets. As another alternative, particularly for veterinary practice, they can be incorporated directly into food. For oral therapeutic administration, they can be incorporated with excipients or used in the form of ingestible tablets, buccal tablets, dragees, pills, troches, capsules, wafers, or other conventional dosage forms.

The tablets, pills, troches, capsules, wafers, or other conventional dosage forms can also contain the following: a binder, such as gum tragacanth, acacia, cornstarch, sorbitol, mucilage of starch, polyvinylpyrrolidone, or gelatin; excipients or fillers such as dicalcium phosphate, lactose, microcrystalline cellulose, or sugar; a disintegrating agent such as potato starch, croscarmellose sodium, or sodium starch glycolate, or alginic acid; a lubricant such as magnesium stearate, stearic acid, talc, polyethylene glycol, or silica; a sweetening agent, such as sucrose, lactose, or saccharin; a wetting agent such as sodium lauryl sulfate; or a flavoring agent, such as peppermint, oil of wintergreen, orange flavoring, or cherry flavoring. When the dosage unit form is a capsule, it can contain, in addition to materials of the above types, a liquid carrier. Various other materials can be present as coatings or to otherwise modify the physical form and properties of the dosage unit. For instance, tablets, pills, or capsules can be coated with shellac, sugar, or both. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical preparations for oral use can be obtained by combining the active compound or compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses or different doses of a single active compound.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycol. In addition, stabilizers may be added.

In one alternative, a sustained-release formulation is used. Sustained-release formulations are well-known in the art. For example, they can include the use of polysaccharides such as xanthan gum and locust bean gum in conjunction with carriers such as dimethylsiloxane, silicic acid, a mixture of mannans and galactans, xanthans, and micronized seaweed, as disclosed in U.S. Pat. No. 6,039,980 to Baichwal. Other sustained-release formulations incorporate a biodegradable polymer, such as the lactic acid-glycolic acid polymer disclosed in U.S. Pat. No. 6,740,634 to Saikawa et al. Still other sustained-release formulations incorporate an expandable lattice that includes a polymer based on polyvinyl alcohol and polyethylene glycol, as disclosed in U.S. Pat. No. 4,428,926 to Keith. Still other sustained-release formulations are based on the Eudragit™ polymers of Rohm & Haas that include copolymers of acrylate and methacrylates with quaternary ammonium groups as functional groups as well as ethylacrylate methylmethacrylate copolymers with a neutral ester group. A particularly-preferred extended release composition suitable for use in methods according to the present invention is an extended-release composition that contains nadolol as its active ingredient.

Oral liquid preparations can be in the form of, for example, aqueous or oily suspensions, solutions, emulsions, syrups, tinctures, or elixirs, or can be presented as a dry product for reconstitution with water or other suitable vehicles before use. Such liquid preparations can contain conventional additives such as suspending agents, for example, sorbitol syrup, methylcellulose, glucose/sugar syrup, gelatin, hydroxymethylcellulose, carboxymethylcellulose, aluminum stearate gel, or hydrogenated edible fats; emulsifying agents, such as lecithin, sorbitan monooleate, or acacia; non-aqueous vehicles (which may include edible oils), for example, almond oil, fractionated coconut oil, oily esters, propylene glycol, or ethyl alcohol; or preservatives, for example, methylparaben, propylparaben, or sorbic acid. The preparations can also contain buffer salts, flavoring, coloring, or sweetening agents (e.g., mannitol) as appropriate.

One skilled in the art recognizes that the route of administration is an important determinant of the rate of efficiency of absorption. For example, the alimentary route, e.g., oral, rectal, sublingual, or buccal, is generally considered the safest route of administration. The delivery of the drugs into the circulation is slow, thus eliminating rapid high blood levels of the drugs that could potentially have adverse acute effects. Although this is considered the safest route of administration, there are several disadvantages. One important disadvantage is that the rate of absorption varies, which is a significant problem if a small range in blood levels separates a drug's desired therapeutic effect from its toxic effect, i.e., if the drug has a relatively low therapeutic index. Also, patient compliance is not always ensured, especially if the rectal route of administration is chosen or if oral administration is perceived by the patient as unpleasant. Furthermore, with oral administration, extensive hepatic metabolism can occur before the drug reaches its target site. Another route of administration is parenteral, which bypasses the alimentary tract. One important advantage of parenteral administration is that the time for the drug to reach its target site is decreased, resulting in a rapid response, which is essential in an emergency. Furthermore, parenteral administration allows for delivery of a more accurate dose. Parenteral administration also allows for more rapid absorption of the drug, which can result in increased adverse effects. Unlike alimentary administration, parenteral administration requires a sterile formulation of the drug and aseptic techniques are essential. The most significant disadvantage to parenteral administration is that it is not suitable for insoluble substances. In addition to alimentary and parenteral administration routes, topical and inhalation administrations can be useful. Topical administration of a drug is useful for treatment of local conditions; however, there is usually little systemic absorption. Inhalation of a drug provides rapid access to the circulation and is the common route of administration for gaseous and volatile drugs, or drugs that can be vaporized or nebulized. It is also a desired route of administration when the targets for the drug are present in the pulmonary system, which is the case for compositions and methods according to the present invention.

A particularly preferable route of administration for nadolol is by inhalation. Typically, administration of nadolol by inhalation comprises administration of a dose administered by use of a pressurized metered dose inhaler (pMDI), dry powder inhaler, or nebulizer; the administration of the dose by inhalation may or may not generate measurable blood levels of nadolol in the range associated by oral dosing. Typically, the inhaled dose will be delivered by pMDI in the range of from about 1% to about 10% of the minimally effective oral dose.

A pressurized metered dose inhaler consists of three major components; the canister which is produced in aluminum or stainless steel by means of deep drawing, where the formulation resides; the metering valve, which allows a metered quantity of the formulation to be dispensed with each actuation; and an actuator (or mouthpiece) which allows the patient to operate the device and directs the aerosol into the patient's lungs. The formulation itself is made up of the drug, a liquefied gas propellant and, in many cases, stabilizing excipients. The actuator contains the mating discharge nozzle and generally includes a dust cap to prevent contamination. To use the inhaler the patient presses down on the top of the canister, with their thumb supporting the lower portion of the actuator. Actuation of the device releases a single metered dose of the formulation which contains the medication either dissolved or suspended in the propellant. Breakup of the volatile propellant into droplets, followed by rapid evaporation of these droplets, results in the generation of an aerosol consisting of micrometer-sized medication particles that are then inhaled. Pressurized metered dose inhalers are disclosed in U.S. Pat. No. 10,806,701 to Bonelli et al.

Dry powder inhalers commonly hold the medication either in a capsule for manual loading or in a proprietary form inside the inhaler. Once the inhaler is loaded or actuated, the operator inserts the mouthpiece of the inhaler into his or her mouth and takes a sharp, deep inhalation, ensuring that the medication reaches the lower parts of the lungs, holding his or her breath for 5-10 seconds. Some powder inhalers use lactose as an excipient. Dry powder inhalers are disclosed in U.S. Pat. No. 10,842,952 to Bilgic.

Nebulizers use oxygen, compressed air, or ultrasonic power to break up solutions or suspensions into small aerosol droplets that are inhaled from the mouthpiece of the device. An aerosol is a mixture of gas and solid or liquid particles. The most common nebulizers are jet nebulizers, sometimes referred to as atomizers. Other forms of nebulizers are soft mist inhalers, ultrasonic wave nebulizers, and vibrating mesh nebulizers. Nebulizers are disclosed in U.S. Pat. No. 10,799,902 to Maeda et al., U.S. Pat. No. 10,786,638 to Alizoti et al., and U.S. Pat. No. 10,716,907 to Eicher et al.

When compounds are formulated for parenteral administration, e.g., formulated for injection via the intravenous, intramuscular, subcutaneous, intralesional, or intraperitoneal routes or other routes known in the art, many options are possible. The preparation of an aqueous composition that contains an effective amount of the nadolol, the derivative or analog of nadolol, or the prodrug of the nadolol or the derivative or analog of nadolol as an active ingredient will be known to those of skill in the art. Typically, such compositions can be prepared as injectables, either as liquid solutions and/or suspensions. Solid forms suitable for use to prepare solutions and/or suspensions upon the addition of a liquid prior to injection can also be prepared. The preparations can also be emulsified. The pharmaceutical forms suitable for injectable use include sterile aqueous solutions and/or dispersions; formulations including sesame oil, peanut oil, synthetic fatty acid esters such as ethyl oleate, triglycerides, and/or aqueous propylene glycol; and/or sterile powders for the extemporaneous preparation of sterile injectable solutions and/or dispersions. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. In all cases the form must be sterile and/or must be fluid to the extent that the solution will pass readily through a syringe and needle of suitable diameter for administration. It must be stable under the conditions of manufacture and storage and must be preserved against the contaminating action of microorganisms, such as bacteria or fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and use, these preparations contain a preservative to prevent the growth of microorganisms. Suitable non-sensitizing and non-allergenic preservatives are well known in the art.

The carrier can also be a solvent and/or dispersion medium containing, for example, water, ethanol, a polyol (for example, glycerol, propylene glycol, and/or liquid polyethylene glycol, and/or the like), suitable mixtures thereof, and/or vegetable oils. The proper fluidity can be maintained for example, by the use of a coating, such as lecithin, by the maintenance of a suitable particle size in the case of a dispersion, and/or by the use of surfactants. The prevention of the action of microorganisms can be brought about by the inclusion of various antibacterial and/or antifungal agents, for example, parabens, chlorobutanol, phenol, sorbic acid, or thimerosal. In many cases it will be preferable to include isotonic agents, for example, sugars or sodium chloride. In many cases, it is preferable to prepare the solution in physiologically compatible buffers such as Hanks's solution, Ringer's solution, or physiological saline buffer. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by sterilization. Sterilization is typically performed by filtration. Generally, dispersions are prepared by incorporating the various sterilized active ingredients into a sterile vehicle which contains the basic dispersion medium and/or the other required ingredients. In the case of sterile powders for the preparation of sterile injectable solutions, the preferred methods of preparation are vacuum-drying and/or freeze-drying techniques that yield a powder of the active ingredients plus any additional desires ingredients from a previously sterile-filtered solution thereof. The preparation of more-concentrated or highly-concentrated solutions for direct injection is also contemplated, where the use of dimethyl sulfoxide (DMSO) as solvent is envisioned to result in extremely rapid penetration, delivering high concentrations of the active agents to a small area if desired.

For parenteral administration in an aqueous solution, for example, the solution should be suitably buffered if necessary and/or the liquid diluent first rendered isotonic with sufficient saline, glucose, or other tonicity agent. These particular aqueous solutions are especially suitable for intravenous, intramuscular, subcutaneous, or intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected into the proposed site of infusion (see, e.g., “Remington's Pharmaceutical Sciences” (15^{th} ed.), pp. 1035-1038, 1570-1580). Some variation in dosage will necessarily occur depending on the condition of the subject being treated. The person responsible for administration will, in any event, determine the appropriate dose for the individual subject. Compounds and compositions according to the invention can also be formulated for parenteral administration by bolus injection or continuous infusion and can be presented in unit dose form, for instance as ampules, vials, small volume infusions, or pre-filled syringes, or in multi-dose containers with an added preservative.

Another route of administration of compositions according to the present invention is nasally, using dosage forms such as nasal solutions, nasal sprays, aerosols, or inhalants. Nasal solutions are usually aqueous solutions designed to be administered to the nasal passages in drops or sprays. Nasal solutions are typically prepared so that they are similar in many respects to nasal secretions, so that normal ciliary action is maintained. Thus, the aqueous nasal solutions usually are isotonic and/or slightly buffered in order to maintain a pH of from about 5.5 to about 6.5. In addition, antimicrobial preservatives, similar to those used in ophthalmic preparations, and/or appropriate drug stabilizers, if required, can be included in the formulation. Various commercial nasal preparations are known and can include, for example, antibiotics or antihistamines. Spray compositions can be formulated, for example, as aqueous solutions or suspensions or as aerosols delivered from pressurized packs, with the use of a suitable propellant, such as dichlorodifluoromethane, trichlorofluoromethane, dichlorotetrafluoroethane, 1,1,1,2,3,3,3-heptafluoropropane, 1,1,1,2-tetrafluoroethane, carbon dioxide, or other suitable gas.

Additional formulations that are suitable for other modes of administration include vaginal suppositories and/or pessaries. A rectal pessary or suppository can also be used. Suppositories are solid dosage forms of various weights or shapes, usually medicated, for insertion into the rectum, vagina, or urethra. After insertion, suppositories soften, melt, and/or dissolve into the cavity fluids. In general, for suppositories, conventional binders or carriers can include polyalkylene glycols, cocoa butter, or triglycerides.

Other dosage forms, including but not limited to liposomal formulations, ointments, creams, lotions, powders, or creams, can alternatively be used. Ointments and creams can, for example, be formulated with an aqueous or oily base with the addition of suitable gelling agents and/or solvents. Such bases, can thus, for example, include water and/or an oil such as liquid paraffin or a vegetable oil such as arachis (peanut) oil or castor oil or a solvent such as a polyethylene glycol. Thickening agents which can be used include soft paraffin, aluminum stearate, cetostearyl alcohol, polyethylene glycols, microcrystalline wax, and beeswax. Lotions can be formulated with an aqueous or oily base and will in general also contain one or emulsifying agents, stabilizing agents, dispersing agents, suspending agents, or thickening agents.

Powders for external application can be formed with the aid of any suitable powder base, for example, talc, lactose, or starch.

Because of the nature of the interaction between inverse agonists and the β-adrenergic receptors with which they interact, the therapeutic response develops gradually over time as the receptor density in the affected tissues increases in response to the administration of inverse agonists. Therefore, in one particularly preferred alternative, the dosage is titrated at the start of administration with gradual increases. In other words, the β-adrenergic inverse agonist, specifically the nadolol, the derivative or analog of nadolol, or the prodrug of the nadolol or the derivative or the analog of the nadolol, is administered over time in a series of graduated doses starting with the lowest dose and increasing to the highest dose. When the highest dose is reached, the β-adrenergic inverse agonist continues to be administered at that dose (the maintenance dose). For example, with nadolol administered orally, treatment can begin with 1 mg dosages, then progress through 3 mg, 5 mg, 10 mg, 15 mg, and then to higher maintenance dosages such as 25 mg, 30 mg, 50 mg, 70 mg, 100 mg, or higher as deemed necessary, depending on the particular condition to be treated, the severity, and the response of the condition to the treatment. In the context of the present invention, the “particular condition to be treated” is typically chronic obstructive pulmonary disease, although analogous dosage regimens can also be used to treat other diseases and conditions as described above.

Accordingly, another aspect of the invention is a blister pack that includes a range of dosages from the lowest initial dose to the highest maintenance dose of the nadolol, the derivative or analog of nadolol, or the prodrug of the nadolol or the derivative or analog of the nadolol. In general, such a blister pack comprises:
(1) a lower substrate;
(2) an intermediate dosage holder that is shaped to generate a plurality of cavities and that is placed over the lower substrate, the cavities being shaped to hold dosage forms of the nadolol, the derivative or analog of nadolol, or the prodrug of the nadolol or the derivative or analog of the nadolol;
(3) an upper substrate placed over the intermediate dosage holder that has a plurality of apertures, each aperture being located to accommodate a corresponding cavity; wherein the dosage forms are of graduated dosages starting with a lowest dose and proceeding to a highest dose; and
(4) dosage forms of the nadolol, the derivative or analog of nadolol, or the prodrug of the nadolol or the derivative or analog of the nadolol, placed in the cavities.

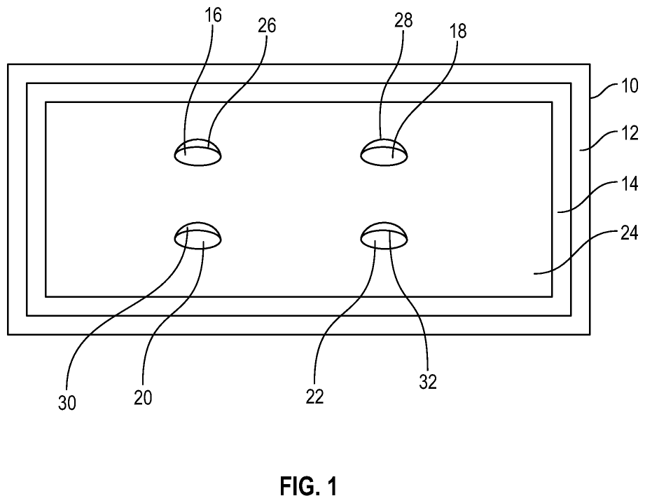

A suitable blister pack 10 is shown in FIG. 1 and includes a lower substrate 12 that is typically foil, an intermediate dosage holder 14 that is shaped to generate a plurality of cavities 16, 18, 20, and 22 shaped to hold the pills, capsules, or other dosage forms that is placed over the lower substrate, and an upper substrate 24 placed over the intermediate dosage holder 14 that has apertures 26, 28, 30, and β₂, each aperture being located to accommodate the cavities 16, 18, 20, and 22. Only four cavities and apertures are shown here, but blister packs 10 according to the present invention can hold a larger number of dosage forms, such as 10, 20, or 30. Typically, either the lower substrate 12, the upper substrate 24, or both have printed instructions on it to identify the dosage of each pill, capsule, or other dosage forms, and to provide guidance to the patient as to the sequence to be followed in taking the pills, capsules, or other dosage forms. The intermediate dosage holder 14 is typically made of a transparent plastic or other transparent material so that the dosage forms can be viewed. The dosage forms can be of graduated doses, starting with a lowest dose and proceeding to a highest dose, which is generally the maintenance dose, as described above. Alternatively, the dosage forms can be of at least two dosages: (1) a maintenance dose that is the highest in a series of graduated doses; and (2) at least one backup restoration dose (to be used, e.g., if a dose is missed) or a lower dose to be taken in a specified condition. The specified condition can be, for example, the administration of an antibiotic, such as erythromycin or neomycin, where lower dosages are generally required or when kidney or liver malfunction increases the half-life of the drug necessitating a lower dose to achieve the same serum concentration when kidney and liver function were both normal.

Various factors must be taken into account in setting suitable dosages for the nadolol, the derivative or analog of the nadolol, or the prodrug of the derivative or the analog of the nadolol. These factors include whether the patient is taking other medications that can alter the pharmacokinetics of the nadolol, the derivative or analog of the nadolol, or the prodrug of the derivative or the analog of the nadolol, either causing them to be degraded more rapidly or more slowly. These medications can, for example, affect either liver or kidney function or may induce the synthesis of one or more cytochrome P450 enzymes that can metabolize the nadolol, the derivative or analog of the nadolol, or the prodrug of the derivative or the analog of the nadolol. In particular, if the patient is taking the antibiotics erythromycin or neomycin, it is typically necessary to decrease the maintenance dose. Another aspect of the invention is therefore a blister pack that has backup restoration doses and lower doses for use when the patient is taking these antibiotics. In another alternative, when the patient is taking a medication that induces the synthesis of one or more cytochrome P450 enzymes that can metabolize the nadolol, the derivative or analog of the nadolol, or the prodrug of the derivative or the analog of the nadolol, a blister pack according to the present invention can include one or more higher doses for temporary use.

Toxicity and therapeutic efficacy of β-adrenergic inverse agonists used in methods and compositions according to the present invention, in particular, nadolol or a derivative or analog of nadolol, or a prodrug of the derivative or the analog of the nadolol, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀; compounds which exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture (i.e., the concentration of the test compound which achieves a half-maximal improvement in receptor signaling when chronic effects are considered). Such information can be used to more accurately determine useful doses in humans. Levels in plasma may be measured, for example, by HPLC or other methods known in the art, such as gas chromatography.

The exact formulation, route of administration and dosage can be chosen by the individual physician in view of the patient's condition. (See, e.g., Fingl et al., in The Pharmacological Basis of Therapeutics, 1975, ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated, such as, but not limited to, chronic obstructive pulmonary disease, and to the route of administration. The severity of the condition may, for example, be evaluated, in part, by standard prognostic evaluation methods. Further, the dose and perhaps the dose frequency, will also vary according to the age, body weight, and response of the individual patient. A program comparable to that discussed above may be used in veterinary medicine.

Depending on the specific conditions being treated, such agents may be formulated and administered systemically or locally. Typically, administration is systemic. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, or intramedullary injections, as well as intrathecal, direct intraventricular, intravenous, intraperitoneal, intranasal, or intraocular injections, just to name a few. Typically, oral administration is preferred for administration of nadolol, derivatives or analogs of nadolol, or prodrugs of the nadolol or the derivatives or analogs of nadolol, for treatment of conditions such as, but not limited to, chronic obstructive pulmonary disease.

For injection, the agents of the invention may be formulated in aqueous solutions. For transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Typically, in methods according to the present invention, the inverse agonist, in particular, the nadolol, the derivative or analog of nadolol, or the prodrug of the nadolol or the derivative or prodrug of the nadolol, is administered in a daily dose or multiple times per day, depending on the half-life of the inverse agonist. Alternatively, the inverse agonist can be administered less frequently, such as every other day, every third day, every fourth day, every week, and the like. One skilled in the art of pharmacokinetics will recognize the importance of understanding the bioavailability and the half-life of a drug in relation to dosing of the particular drug. It is well known that a drug accumulates in the body if the time interval between doses is less than four of its half-lives, in which case, the total body stores of the drug are increased exponentially to a plateau or steady-state concentration. The average total body store of a drug at the plateau is a function of the dose, the interval between doses, the bioavailability of the drug, and the rate of the elimination of the drug. Thus, one of ordinary skill in the art is capable of determining the dose and interval of the dose for a given drug to achieve the desired effect.

Another embodiment of the present invention is methods and compositions that incorporate multiple-drug or combination therapy for the treatment of pulmonary airway diseases, in particular, for the treatment of chronic obstructive pulmonary disease. Patients with pulmonary airway diseases often are prescribed multiple drugs that work in combination to control their symptoms.

Although Applicant does not intend to be bound by this theory, it is believed that, in many circumstances, co-treatment with an inverse agonist, particularly the nadolol, the derivative or the analog of the nadolol, or the prodrug of the nadolol or the derivative or the analog of the nadolol, and with an agonist is superior to treatment with the agonist alone. These results suggest that co-treatment with the inverse agonist may increase the therapeutic efficacy of the agonist and prevent desensitization of the relevant GPCR. One rationale for this form of combination therapy may lie in the treatment of acute episodes or exacerbations of the disease or condition. Even if treatment with inverse agonists decreases the frequency of such acute episodes or exacerbations, there is still a need to treat the acute episode or exacerbation. This can be done by co-administration of the inverse agonist and the agonist.

In one particularly desirable combination, the β-adrenergic inverse agonist is administered in combination with β₂-selective adrenergic agonists for the treatment of pulmonary airway diseases. The 2-selective adrenergic agonists are typically selected from the group consisting of abediterol, arformoterol, bambuterol, bitolterol, carmoterol, clenbuterol, clorprenaline, dobutamine, fenoterol, formoterol, indicaterol, isoprenaline, isoxsuprine, levabuterol, mabuterol, metaproterenol, olodaterol, pirbuterol, procaterol, ritodrine, salbutamol, salmeterol, terbutaline, vilanterol, and zilpaterol, as well as the salts, solvates, and prodrugs thereof. These agents vary with respect to their duration of action, generally being classified as short-acting, long-acting, or ultra-long-acting agents. Particularly preferred β₂-selective adrenergic agonists for use in combination with nadolol include isoproterenol, salbutamol, and salmeterol. The principle of combination therapy is supported by the data that shows that treatment with inverse agonists causes upregulation of the receptor number. In that case, co-treatment with an agonist would be expected to increase cellular signaling and restore normal function in those circumstances in which the pathological response is characterized by a deficiency in signaling. Along these lines, the inhibitory response of inverse agonists on airway resistance would be increased in magnitude by the co-administration of agonists. The potency of these agonists may be reduced due to the presence of the inverse agonist, but the overall magnitude of the response would be increased. This would prevent the desensitization often associated with chronic agonist treatment.

When combination therapy is used, the dosages of each member of the combination can be determined according to the principles described above. However, in many cases, fixed dose combinations are desirable and can be used. In the fixed dose combinations, the dosages of the β-adrenergic inverse agonists are as described above, while the desirable dosage of the 2-selective adrenergic agonist can be determined as described above. The determination of the dosages of each member of the combination or the use of fixed dose combinations also apply to other combinations of β-adrenergic inverse agonists and other therapeutic agents as described below.

In another desirable combination, β-adrenergic inverse agonists are administered together with corticosteroids. The corticosteroids especially preferred for use according to the invention include, but are not necessarily limited to, beclomethasone, budenoside, ciclesonide, flunisolide, fluticasone, methylprednisolone, prednisolone, prednisone, dexamethasone, and triamcinolone, as well as the salts, solvates, and prodrugs thereof.

In yet another desirable combination, β-adrenergic inverse agonists are administered together with anticholinergics. The anticholinergics especially preferred for use according to the invention include, but are not necessarily limited to, muscarinic receptor antagonists, especially quaternary ammonium muscarinic receptor antagonists such as ipratropium bromide, tiotropium bromide, oxitropium bromide, aclidinium bromide, glycopyrronium bromide, umeclidinium bromide, as well as the salts, solvates, and prodrugs thereof.

In yet another desirable combination, β-adrenergic inverse agonists are administered together with a xanthine compound. Xanthine compounds especially preferred for use according to the invention include, but are not necessarily limited to, theophylline, extended-release theophylline, aminophylline, theobromine, enprofylline, diprophylline, isbufylline, choline theophyllinate, albifylline, arofylline, bamifylline, ambuphylline, 8-chlorotheophylline, doxofylline, furafylline, IBMX (1-methyl-3-(2-methylpropyl)-7H-purine-2,6-dione), MRS-1706 (N-(4-acetylphenyl)-2-[4-(2,3,6,7-tetrahydro-2,6-dioxo-1,3-dipropyl-1H-purin-8-yl)phenoxy]acetamide), proxyphylline, and caffeine, as well as the salts, solvates, and prodrugs thereof.

In yet another desirable combination, β-adrenergic inverse agonists are administered together with an anti-IgE antibody. As used herein, unless further defined or limited, the term “antibody” encompasses both polyclonal and monoclonal antibodies, as well as genetically engineered antibodies such as chimeric, humanized or fully human antibodies of the appropriate binding specificity. As used herein, unless further defined, the term “antibody” also encompasses antibody fragments such as sFv, Fv, Fab, Fab′ and F(ab)′₂ fragments. In many cases, it is preferred to use monoclonal antibodies. In some contexts, antibodies can include fusion proteins comprising an antigen-binding site of an antibody, and any other modified immunoglobulin molecule comprising an antigen recognition site (i.e., antigen-binding site) as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins, therapeutic agents, antimetabolites, or radioisotopes, in some cases, conjugation occurs through a linker or through noncovalent interactions such as an avidin-biotin or streptavidin-biotin linkage. Typically, the anti-IgE antibody is a monoclonal antibody or a genetically engineered antibody that is derived from a monoclonal antibody. Preferably, the anti-IgE antibody is humanized. A particularly preferred humanized anti-IgE antibody is an IgG1λ monoclonal antibody that specifically binds to human IgE and is marketed under the name of omalizumab.

In yet another desirable combination, β-adrenergic inverse agonists are administered together with a leukotriene antagonist. The leukotriene antagonists especially preferred for use according to the present invention include, but are not necessarily limited to, montelukast, pranlukast, and zafirlukast, as well as the salts, solvates, and prodrugs thereof.

In yet another desirable combination, β-adrenergic inverse agonists are administered together with a phosphodiesterase IV inhibitor. The phosphodiesterase IV inhibitors especially preferred according to the present invention include, but are not necessarily limited to, roflumilast, cilomilast, piclamilast, and ibudilast, as well as the salts, solvates, and prodrugs thereof. Phosphodiesterase IV is the predominant isoform in the lung and inhibitors of this enzyme are being considered for the treatment of asthma and COPD.

In yet another desirable combination, β-adrenergic inverse agonists are administered together with a 5-lipoxygenase inhibitor. The 5-lipoxygenase inhibitors especially preferred according to the present invention include, but are not limited to, zileuton and fenleuton, as well as the salts, solvates, and prodrugs thereof.

In yet another desirable combination, β-adrenergic inverse agonists are administered together with a mast cell stabilizer. The mast cell stabilizers especially preferred according to the present invention include, but are not limited to, azelastine, cromoglicic acid, ketotifen, lodoxamide, nedocromil, olopatadine, and pemirolast, as well as the salts, solvates, and prodrugs thereof.

In yet another desirable combination, β-adrenergic inverse agonists are administered together with a biological (B. L. Walker & R. Leigh, “Use of Biologicals as Immunotherapy in Asthma and Related Diseases,” Expert Rev. Clin. Immunol. 4: 753-756 (2008)). Typically, the at least one biological is selected from the group consisting of an anti-IL4 antibody, an anti-IL13 antibody, an inhibitor of both IL4 and IL13, an anti-IL5 antibody, and an anti-IL8 antibody. Anti-IL4 antibodies include, but are not limited to, a humanized anti-IL4 monoclonal antibody, pasolizumab. Anti-IL13 antibodies include, but are not limited to, a human anti-IL13 monoclonal antibody, CAT-354. An inhibitor of both IL4 and IL13 is the monoclonal antibody dupilumab, which is a monoclonal antibody that binds to the a subunit of the interleukin-4 receptor (IL4Ra), which modulates the signaling of both the IL4 and the IL13 pathways; it therefore acts as a receptor antagonist (A. L. Kau & P. E. Korenblat, “Anti-Interleukin 4 and 13 for Asthma Treatment in the Era of Endotypes,” Curr. Opin. Allergy Clin. Immunol. 14: 570-575 (2014)). Anti-IL5 antibodies include, but are not limited to, the monoclonal antibodies benralizumab, mepolizumab, and reslizumab. Anti-IL8 antibodies include, but are not limited to, the human monoclonal antibody BMS-986253.

The route of administration of the β-adrenergic inverse agonist and of the additional therapeutic agent can be chosen by one of ordinary skill in the art to optimize therapeutic efficiency, as described above. However, in one preferred alternative, both the β-adrenergic inverse agonist and the additional therapeutic agent are administered by inhalation. In another preferred alternative, the β-adrenergic inverse agonist is administered orally, while the additional therapeutic agent is administered by inhalation. The administration of the additional therapeutic agent by inhalation is typically preferred because of possible toxicity of some of these additional therapeutic agents. However, other routes are possible.

Aerosol therapy allows an almost ideal benefit to risk ratio to be achieved because very small doses of inhaled medication provide optimal therapy with minimal adverse effects. A variety of additional therapeutic agents suitable for use in methods according to the present invention are available in aerosol formulation, including β₂-adrenergic agonists, corticosteroids, and anticholinergics. However, the therapeutic efficiency of drugs administered by aerosolization depends not only on the pharmacological properties of the drugs themselves, but also on the characteristics of the delivery device. The characteristics of the delivery device influence the amount of drug deposited in the lungs and the pattern of drug distribution in the airways.

Aerosols are airborne suspensions of fine particles. The particles may be solids or liquids. Aerosol particles are heterodisperse (i.e. the particles are of a range of sizes) and aerosol particle size distribution is best described by a log normal distribution. Particles tend to settle (sediment), adhere to each other (coagulate), and adhere to structures such as tubing and mucosa (deposit). The particles delivered by aerosol can be conveniently characterized on the basis of their aerodynamic behavior. One parameter is the mass median aerodynamic diameter (MMAD). By definition, a particle distribution with an MMAD of 1 μm has the same average rate of settling as a droplet of unit density and 1 μm diameter.

The size of an aerosol particle, as well as variables affecting the respiratory system, influence the deposition of inhaled aerosols in the airways. On one hand, particles larger than 10 μm in diameter are unlikely to deposit in the lungs. However, particles smaller than 0.5 μm are likely to reach the alveoli or may be exhaled. Therefore, particles that have a diameter of between 1 μm and 5 μm are most efficiently deposited in the lower respiratory tract. Particles of these sizes are most efficient for the delivery of therapeutic agents for diseases and conditions such as chronic obstructive pulmonary diseases.

The percentage of the aerosol mass contained within respirable droplets (i.e., droplets with a diameter smaller than 5 μm), depends on the inhalation device being used. Slow, steady inhalation increases the number of particles that penetrate the peripheral parts of the lungs. As the inhaled volume is increased, the aerosol can penetrate more peripherally into the bronchial tree. A period of breath-holding, on completion of inhalation, enables those particles that have penetrated to the lung periphery to settle into the airways via gravity. Increased inspiratory flow rates, typically observed in patients with acute asthma, result in increased losses of inhaled drug. This occurs because aerosol particles impact in the upper airway and at the bifurcations of the first few bronchial divisions. Other factors associated with pulmonary airway disease may also alter aerosol deposition. Airway obstruction and changes in the pulmonary parenchyma are often associated with pulmonary deposition in the peripheral airways in patients with asthma or other diseases or conditions affecting the respiratory tract, such as chronic obstructive pulmonary disease.

In aerosol administration, the nose efficiently traps particles before their deposition in the lung; therefore, mouth breathing of the aerosolized particles is preferred. The aerosolized particles are lost from many sites. Generally, the amount of the nebulized dose reaching the small airways is ≤15%. In many cases, approximately 90% of the inhaled dose is swallowed and then absorbed from the gastrointestinal tract. The small fraction of the dose that reaches the airways is also absorbed into the blood stream. The swallowed fraction of the dose is, therefore, absorbed and metabolized in the same way as an oral formulation, while the fraction of the dose that reaches the airways is absorbed into the blood stream and metabolized in the same way as an intravenous dose.

When drugs are administered topically (via aerosol delivery to the lungs), the desired therapeutic effects depend on local tissue concentrations, which may not be directly related to plasma drug concentrations. If a sufficiently large dosage of any drug is given, systemic activity can easily be demonstrated with any inhaled (β₂-agonists or corticosteroid. This has several implications. First, for the selection of a drug to be inhaled, topical drugs must combine a high intrinsic activity within the target organ and rapid inactivation of the systemically absorbed drug. Secondly, fewer systemic adverse effects should be expected with drugs that have a low oral bioavailability (whether due to poor gastrointestinal absorption or high first-pass hepatic metabolism). Because most inhaled drugs are administered at a low dosage and have a low oral bioavailability, plasma concentrations of these drugs are much lower than after oral administration. Furthermore, factors influencing pulmonary absorption should be considered. It was recently demonstrated that terbutaline was absorbed through the lung more rapidly in healthy smokers than in healthy nonsmokers. This may affect the onset of action of the drug. It has also been found that the bioavailability of inhaled salbutamol in 10 patients with cystic fibrosis was greater than that in healthy adults. One proposed mechanism for this difference in bioavailability is that the chronically diseased tracheobronchial tree in patients with cystic fibrosis results in higher permeability of salbutamol in this tissue.

Finally, the absolute pulmonary bioavailability of inhaled drugs is difficult to assess because blood concentrations are low, and pulmonary and oral absorption should be discriminated for pulmonary bioavailability to be determined as accurately as possible. Charcoal can be used to adsorb the swallowed fraction of inhaled terbutaline to discriminate the pulmonary absorption of the drug. Recently, it was shown that a urine collection during the 30 minutes after inhalation of salbutamol represents the amount of drug delivered to the lungs. This technique may be applicable for the determination of bioavailability of other inhaled drugs. Other techniques for the determination of bioavailability of inhaled drugs are also known in the art; these include pharmacodynamic methods using FEV1 measurements, lung deposition studies using radiolabeled formulations, or pharmacokinetic studies using predominantly urinary excretion measurements.

Therapeutic aerosols are commonly produced by atomization of liquids within jet nebulizers or by vibration of a standing pool of liquid (ultrasonic nebulization). Preformed aerosols may also be administered. Examples of the latter include MDIs and dry powder devices. Whatever delivery device is used, patients should be taught to use it correctly.

All jet nebulizers work via a similar operating principle, represented by the familiar perfume atomizer. A liquid is placed at the bottom of a closed container, and the aerosol is generated by a jet of air from either a compressor or a compressed gas cylinder passing through the device. Ultrasonic nebulizers produce an aerosol by vibrating liquid lying above a transducer at frequencies of about 1 mHz. This produces a cloud of particles that is carried out of the device to the patient by a stream of air. Aerosols varying in quantity, size and distribution of panicles can be produced by nebulizers, depending upon the design of the nebulizers and how it is operated. It should be noted that not all nebulizers have the required specifications (MMAD, flow, output) to provide optimum efficacy. A recent study compared the lung deposition from 4 nebulizers in healthy volunteers and showed that median lung aerosol deposition, expressed as percentages of the doses initially loaded into the nebulizers, ranged from 2 to 19%. Nebulized aerosols are particularly useful for children under 5 years of age and in the treatment of severe asthma or chronic obstructive pulmonary disease where respiratory insufficiency may impair inhalation from an MDI or dry powder inhaler. To minimize adverse effects, pH and osmolarity of the nebulized solution should be controlled.

Metered dose inhalers (MDIs), because of their convenience and effectiveness, are probably the most widely used therapeutic aerosol used for inhaled drug delivery to outpatients. Most MDIs in current use contain suspensions of drug in propellant. There are 2 major components of an MDI: (i) the canister, a closed plastic or metal cylinder that contains propellant, active medication, and the metering chamber; and (ii) the actuator, a molded plastic container that holds the canister and directs the released aerosol towards the patient's airway.

Propellant mixtures are selected to achieve the vapor pressure and spray characteristics desired for optimal drug delivery. Chlorofluorocarbons were previously used, but non-chlorinated propellants are now employed because of environmental concerns. Finely divided particles of drug, usually less than 1 μm, are suspended in the pressurized (liquefied) propellant. To prevent the drug from coagulating, a surface-active agent such as sorbitan oleate, lecithin or oleic acid is typically added; other surface-active agents are known in the art. Metering chambers ordinarily contain 25 to 100 μL. The contents of the metering chamber are released when the canister is depressed into the actuator. Almost instantaneously, the propellants begin to evaporate, producing disintegration of the discharged liquid into particles that are propelled forward with great momentum. For optimal pulmonary drug deposition, the medication should be released at the beginning of a slow inspiration that lasts about 5 seconds and is followed by 10 seconds of breath-holding. Several inhalation aids have been designed to improve the effectiveness of an MDI. These are most useful in patients who have poor hand-to-breath coordination. A short tube (e.g. cones or spheres) may direct the aerosol straight into the mouth or collapsible bags may act as an aerosol reservoir holding particles in suspension for 3 to 5 seconds, during which time the patient can inhale the drug. However, when any of these devices is used, aerosol velocity upon entering the oropharynx is decreased and drug availability to the lungs and deposition in the oropharynx is decreased.

Dry powder inhalers have been devised to deliver agents to patients who have difficulty using an MDI (such as children and elderly patients). In general, the appropriate dosage is placed in a capsule along with a flow aid or filler such as large lactose or glucose panicles. Inside the device, the capsule is initially either pierced by needles (e.g., Spinhaler®) or sheared in half (e.g., Rotohaler®). During inhalation the capsule rotates or a propeller is turned, creating conditions that cause the contents of the capsule to enter the inspired air and be broken up to small particles suitable for delivery to the airways. The energy required to disperse the powder is derived from the patient's inspiratory effort. Recently, more convenient multidose dry powder inhalers have been introduced (e.g. Diskhaler®, Turbuhaler®). Potential problems associated with dry powder inhalers include esophageal irritation and, consequently, cough due to the direct effect of powder in airways. Furthermore, the walls of the capsule may be coated with drug as a result of either failure of the capsule to release the drug or failure of the aggregated powder to break up. This may cause virtually all of the drug to be deposited in the mouth. These powder devices do not contain chlorofluorocarbons and may provide an alternative to MDIs.

The clinical use of aerosols for treatment of diseases and conditions affecting the respiratory tract such as, but not limited to, chronic obstructive pulmonary disease and asthma as well as other diseases and conditions treatable by methods and compositions according to the present invention, has been proposed for several compounds proposed herein as additional therapeutic agents, including 2-agonists and corticosteroids.

For β₂-agonists, limited pharmacokinetic data are available in humans mostly because the low dosages of inhaled drugs required for therapeutic activity produce drug concentrations in body fluids that are below assay limits. Little is known about pulmonary bioavailability of those drugs. It is generally argued that an average of 10% of an inhaled dose reaches the lung when given by a MDI. The mean pulmonary bioavailability of terbutaline from an MDI was reported to be 9.1%. When the oral component (swallowed fraction of the dose) was added, the value rose to 16.5%, i.e., an increase of 6.7%. The drugs salmeterol and formoterol have different mechanisms of action underlying their prolonged duration of bronchodilatory effect (12 to 18 hours). Salmeterol appears unique because it has a long side chain that anchors the β₂-agonist molecule to the receptor. Formoterol appears to be an extremely potent classical β₂-agonist. The elimination half-life of formoterol after inhalation was calculated to be between 1.7 and 2.3 hours on the basis of urinary excretion data. A glucuronic acid conjugate was identified. However, it is possible that formoterol has a prolonged elimination half-life that is yet to be detected in humans. Salmeterol is formulated as the xinafoate (hydroxynaphthoic acid) salt. Little is known about the pharmacokinetic properties of this drug. Salmeterol is extensively metabolized by hydroxylation, with the majority of a dose being eliminated predominantly in the feces within 72 hours. The hydroxynaphthoic acid part of the molecule accumulates in plasma during repeated administration as a consequence of its long elimination half-life (12 to 15 days).

For anticholinergic agents, the parent compound of this class is atropine. Synthetic agonists of the muscarinic receptors of acetylcholine are quaternary ammonium compounds and, therefore, cross membrane barriers with difficulty. Because systemic absorption of atropine after inhalation of the drug is nearly complete, this route of administration can produce significant systemic toxicity (Harrison et al. 1986). Ipratropium bromide is the only well studied representative of this class. Absorption through the gastrointestinal tract is slow, as peak plasma concentrations have been recorded 3 hours after oral intake of the drug. The absolute bioavailability after oral intake is only 30%. Elimination of metabolized drug occurs in the urine and bile. Whatever the route of administration, the mean elimination half-life is about 3.5 hours. Plasma concentrations observed with inhaled ipratropium were a thousand times lower than those observed with an equipotent bronchodilatory dose administered orally. This explains why systemic anticholinergic effects do not occur following inhalation of therapeutic doses of ipratropium bromide. These properties are probably shared by other quaternary ammonium anticholinergic agents such as oxitropium bromide, an alternative as described above.

Corticosteroids are frequently administered by inhalation, which can prevent some of the adverse effects usually associated with systemic corticosteroid therapy. To produce a compound with marked topical activity, some of the hydroxyl groups in the hydrocortisone molecule were substituted with acetonide or ester groups. Topically active corticosteroid drugs used for the treatment of patients with asthma include beclomethasone, betamethasone valerate, budesonide, triamcinolone, fluticasone and flunisolide. Of these, beclomethasone and budesonide are the most extensively used. The results of numerous clinical studies have shown that there is little difference between the efficacy of beclomethasone and budesonide. Oropharynx deposition is reduced by using a spacing device, and the development of candidiasis can be prevented by mouth rinsing. Plasma clearance of budesonide was calculated to be 84±27 L/h, which is about 10-fold higher than the average clearance of prednisolone. As a consequence of this high clearance, the elimination half-life of budesonide is short (2.8±1.1 hours). The systemic availability of the swallowed fraction is 10.7±4.3%, indicating that there is extensive first-pass metabolism. Stereoselective metabolism was demonstrated and plasma clearance of the two enantiomers, when calculated on a per kilogram of bodyweight basis, were about 50% higher in 6 children with asthma than in 11 healthy adults. Therefore, administration of budesonide by inhalation should reduce the risk of systemic adverse effects compared with administration of the drug orally. Lung esterases are known to hydrolyze beclomethasone. The absorbed beclomethasone and esterase-hydrolysis products (beclomethasone 17-propionate and beclomethasone) are rapidly converted to less active metabolites during passage through the liver. First-pass hepatic metabolism of the systemically absorbed fluticasone is almost complete, and therefore the inhaled drug has a favorable pharmacokinetic profile. Little data has been published regarding the pharmacokinetic properties of flunisolide, triamcinolone, and betamethasone valerate.

To ensure maximal effects from inhaled drugs, both the pharmacological characteristics of the drugs and the device used to aerosolize the drugs should be considered. With respect to β₂-agonists, different formulations, with different pulmonary disposition techniques, are available, such as for MDI administration, for administration with a dry powder inhaler, or a solution for nebulization. A unit dose from a dry powder inhaler is twice that release from an MDI, but they have equivalent bronchodilatory effects. The characteristics of the devices vary. For a metered-dose inhaler, typically 12-40% of the dose is deposited in the lung, but up to 80% in the oropharynx. When an MDI is used with a spacer, typically about 20% of the dose is deposited in the lung, but only up to 5% in the oropharynx; thus, the use of a spacer can reduce the proportion of the drug that is deposited in the oropharynx. For a dry powder inhaler, typically 11-16% of the dose is deposited in the lung and 31-72% in the oropharynx. For a nebulizer, typically 7-32% of the dose is deposited in the lung and 1-9% is deposited in the oropharynx. One of ordinary skill in the art can ensure that the proper inhalation therapy device is used and can prepare suitable instructions. Considerations for the use of inhalation therapy are described in A.-M. Tabaret & B. Schmit, “Pharmacokinetic Optimisation of Asthma Treatment,” Clin. Pharmacokinet. 26: 396-418 (1994).

For all of these combinations, the invention further encompasses blister packs that contain either a fixed-dose combination of the β-adrenergic inverse agonist and the additional therapeutic agent, such as the 2-selective adrenergic agonist, the corticosteroid, the anticholinergic agent, the xanthine compound, the anti-IgE antibody, the leukotriene antagonist, the phosphodiesterase-4 inhibitor, the 5-lipoxygenase inhibitor, the mast cell stabilizer, or the biological; or, in separate pills, capsules, or other dosage forms, the β-adrenergic inverse agonist and the additional therapeutic agent as described above. The use of these blister packs is appropriate when oral administration of the inverse agonist and additional therapeutic agent is desired. The blister packs follow the general design described above and in FIG. 1, and typically include appropriate instructions to the patient.

In general, when a fixed-dose combination is used, the blister pack comprises:
(1) a lower substrate;
(2) an intermediate dosage holder that is shaped to generate a plurality of cavities and that is placed over the lower substrate, the cavities being shaped to hold dosage forms of the pharmaceutical composition described above containing a β-adrenergic inverse agonist and at least one additional therapeutic agent as described above;
(3) an upper substrate placed over the intermediate dosage holder that has a plurality of apertures, each aperture being located to accommodate a corresponding cavity; and;
(4) dosage forms of the pharmaceutical composition placed in the cavities.

When the β-adrenergic inverse agonist and the additional therapeutic agent are to be administered in separate dosage forms, the blister pack, in general, comprises:
(1) a lower substrate;
(2) an intermediate dosage holder that is shaped to generate a plurality of cavities and that is placed over the lower substrate, the cavities being shaped to hold dosage forms of: (a) a first pharmaceutical composition that comprises: (i) a therapeutically effective amount of a β-adrenergic inverse agonist; and (ii) a first pharmaceutically acceptable carrier; and (b) a second pharmaceutical composition that comprises: (i) a therapeutically effective amount of a second therapeutic agent effective to treat a pulmonary airway disease, the second therapeutic agent being selected from the group consisting of a 2-selective adrenergic agonist, a corticosteroid, a anticholinergic agent, a xanthine compound, an anti-IgE antibody, a leukotriene antagonist, a phosphodiesterase-4 inhibitor, a 5-lipoxygenase inhibitor, a mast cell stabilizer, and a biological and (ii) a second pharmaceutically acceptable carrier;
(3) an upper substrate placed over the intermediate dosage holder that has a plurality of apertures, each aperture being located to accommodate a corresponding cavity; and;
(4) dosage forms of the first and second pharmaceutical compositions placed in the cavities.

The dosage forms of the first and second pharmaceutical compositions are as described above. Typically, in this arrangement, the dosage forms of the first pharmaceutical composition include dosages starting at a low dose and including a range of dosages up to the highest, maintenance, dose. Other dosage form arrangements are possible.

Other arrangements are possible for the blister packs.

In yet another alternative according to the present invention, the nadolol, the derivative or analog of nadolol, or the prodrug of the nadolol or the derivative or analog of the nadolol is administered together with a therapeutically effective quantity of an arrestin-2 inhibitor. As used herein, the term “arrestin-2 inhibitor” refers to any compound that directly or indirectly blocks one or more effects of arrestin-2 on β-adrenergic receptors, particularly β₂-adrenergic receptors, and thus potentiates the activity of such receptors when bound to agonists.

U.S. Pat. No. 10,172,907 to Lymperopoulos et al. discloses protein fragments of arrestin-2 as arrestin-2 inhibitors, particularly a fragment with the sequence: Glu-Thr-Pro-Val-Asp-Thr-Asn-Leu-Ile-Glu-Leu-Asp-Thr-Asn-Asp-Asp-Asp-Ile-Val-Phe-Glu-Asp-Phe-Ala-Arg-Gln-Arg-Leu-Lys-Gly-Met-Lys-Asp-Asp-Lys-Asp-Glu-Glu-Asp-Asp-Gly-Thr-Gly-Ser-Pro-His-Leu-Asn-Asn-Arg (SEQ ID NO: 1).

U.S. Pat. No. 9,926,275 to Thakur et al. discloses small molecule inhibitors of arrestin-2, including compounds of Formula (A-I):

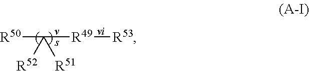

wherein:
(1) v and vi designate the particular bonds indicated in Formula (A-I);
(2) R⁴⁹ is selected from Formulas (A-I(a)), (A-I(b)), (A-I(c)), and (A-I(d)):

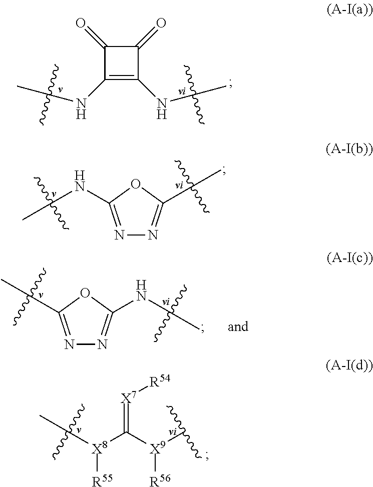

wherein X⁷, X⁸, and X⁹ are each independently O, N, or S;
(2) R⁵⁴, R⁵⁵, and R⁵⁶ are each independently H, cyano, amino, or a substituted or unsubstituted alkyl, alkanoyl, alkanoyloxy, or aryl group when X⁷, X⁸, or X⁹ are respectively N and are absent when X⁷, X⁸, or X⁹ are respectively O or S;
(3) R⁵⁰ is a substituted or unsubstituted aryl or heteroaryl group;
(4) R⁵¹ and R⁵² are each independently H or a substituted or unsubstituted alkyl group, or R⁵¹ and R⁵² together form a 3- or 4-membered cycloalkyl ring;
(5) R⁵³ is a substituted aryl group where one and only one of the substituents is a moiety of Formula (A-I(e)):

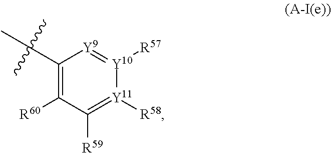

(i) wherein one of R⁵⁷ or R⁵⁸ is a moiety of Formula (A-I(f)):

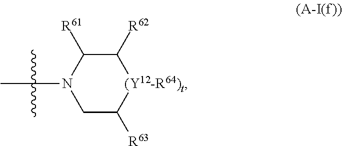

and the other is H, azido, trifluoromethyldiazirido, isocyano, isothiocyano, pentafluorosulfanyl, or a substituted or unsubstituted alkyl, alkanoyl, alkanoyloxy, aryloyl, or aryloyloxy group;
(ii) R⁵⁹ and R⁶⁰ are each independently H, halo, azido, trifluoromethyldiazirido, isocyano, isothiocyano, pentafluorosulfuryl, or a substituted or unsubstituted alkyl, alkanoyl, alkanoyloxy, aryloyl, or aryloyloxy group;
(iii) Y⁹ is CH;
(iv) Y¹⁰ and Y¹¹ are each independently C or N, provided that when Y¹⁰ or Y¹¹ is N then R⁵⁷ or R⁵⁸ respectively is absent;
(v) Y¹² is CH, N, O, S, S(O), or S(O)₂;
(vi) R⁶¹, R⁶², and R⁶³ are each independently H, azido, trifluoromethyldiazirido, isocyano, isothiocyano, or a substituted or unsubstituted alkyl group;
(vii) R⁶⁴ is H or a substituted or unsubstituted alkyl group when Y¹² is CH or N and is absent when Y¹² is O, S, S(O), or S(O)₂; and
(viii) t is 0 or 1; and
(6) s is 0 or 1.

U.S. Pat. No. 8,987,332 to Olefsky et al. discloses omega-3 fatty acids as inhibitors of arrestin-2, including DHA (docosahexaenoic acid) and EPA (eicosapentaenoic acid).

United States Patent Application Publication No. 2016/0311911 by Sur et al., discloses that CXCR2 inhibitors can inhibit the activity of arrestin-2 due to the interaction between CXCR2 and arrestin. CXCR2 inhibitors include, but are not limited to: SB225002 (N-(2-bromophenyl)-N′-(2-hydroxy-4-nitrophenyl)urea), AZD5069 (N-(2-((2,3-difluorobenzyl)thio)-6-(((2R,3S)-3,4-dihydroxybutan-2-yl)oxy)pyrimidin-4-yl)azetidine-1-sulfonamide); SB265610 (1-(2-bromophenyl)-3-(4-cyano-1H-benzo[d][1,2,3]triazol-7-yl)urea); navarixin; danirixin; CXCR2-IN-1 (1-(2-chloro-3-fluorophenyl)-3-[4-chloro-2-hydroxy-3-(1-methylpiperidin-4-yl)sulfonylphenyl]urea); SRT3109 (N-(2-((2,3-difluorobenzyl)thio)-6-((3,4-dihydroxybutan-2-yl)amino)pyrimidin-4-yl)azetidine-1-sulfonamide); and SRT3190 (N-[2-[(2,3-difluorophenyl)methylsulfanyl]-6-[[(2S,3R)-3,4-dihydroxybutan-2-yl]amino]pyrimidin-4-yl]azetidine-1-sulfonamide); MyD88 inhibitors, including, but not limited to: ST2825 ((4R,7R,8aR)-1′-[2-[4-[[2-(2,4-dichlorophenoxy)acetyl]amino]phenyl]acetyl]-6-oxospiro[3,4,8,8a-tetrahydro-2H-pyrrolo[2,1-b][1,3]thiazine-7,2′-pyrrolidine]-4-carboxamide) and T6167923 (4-(3-bromophenyl)sulfonyl-N-(1-thiophen-2-ylethyl)piperazine-1-carboxamide); and MD2 inhibitors, including, but not limited to L48H37 ((3E,5E)-1-ethyl-3,5-bis[(2,3,4-trimethoxyphenyl)methylidene]piperidin-4-one).

United States Patent Application 2004/0053852 by Stamler et al. discloses methods for preventing desensitization of G-protein coupled receptors. These G-protein coupled receptors (GPCRs) include β-adrenergic receptors as well as α-adrenergic receptors, opioid receptors, and prostaglandin receptors. The GPCRs have G-protein receptor kinases (GRKs) associated with them. The GRKs phosphorylate agonist-occupied receptors, thereby promoting binding of β-arrestin molecules, including arrestin-2, which inhibit interactions between the receptors and their associated G-proteins, while also promoting internalization of the receptors. The activity of GRKs thus dampens signaling by the GPCRs. The typical response is decreased levels of GPCRs and the desensitization of the GPCRs, in other words, inability of the agonist that normally binds to the specific GPCR to activate the receptor, which, in certain circumstances, can lead to the inability of the GPCRs to control a disease event associated with lack of activity of a particular GPCR or multiple GPCRs. Nitric oxide donors (NO donors) that donate nitric oxide or a related redox species and provide bioactivity that is identified with nitric oxide, preferably S-nitrosoglutathione (GSNO) inhibit the activity of GRKs thereby allowing GPCRs to signal and to be recycled to the cell surface. This prevents desensitization of the GPCRs and thereby allows GPCRs to be available and active in sufficient quantity to control a disease event that is associated with the lack of activity of a particular GPCR or multiple GPCRs.

NO donors include C-nitroso compounds in which the nitroso moiety is attached to a tertiary carbon, such as the compounds disclosed in U.S. Pat. No. 6,359,182 to Stamler et al. These compounds include C-nitroso compounds having a molecular weight ranging from about 225 to about 1000, or from about 225 to about 600 for oral administration, on a monomeric basis wherein a nitroso group is attached to a tertiary carbon, which is obtained by nitrosylation of a carbon acid having a pKa of less than about 25. The compound is preferably water-soluble and preferably contains a carbon atom a to the nitrosylated carbon which is part of a ketone group. In some alternatives, the compound is obtained by nitrosylation of a carbon acid having a pKa of less than 10, and, for such compounds, the activity can be potentiated by glutathione. In one alternative, for such compounds, a substituent Q is attached to the tertiary carbon and consists of a chain moiety containing from 1 to 12 chain atoms consisting of 1 to 10 carbon atoms, 0 to 2 nitrogen atoms, and 0 to 2 oxygen atoms covalently bonded to a cyclic moiety which is monocyclic, bicyclic, tricyclic, tetracyclic, or pentacyclic, and contains 5 to 24 ring atoms, consisting of 2 to 20 carbon atoms, 0 to 4 nitrogen atoms, 0 to 1 oxygen atoms, and 0 to 1 sulfur atoms.

In another alternative, the C-nitro compound is a compound of Formula (N-I):

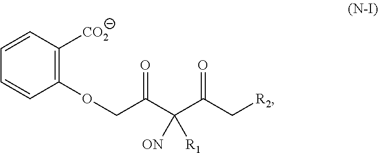

wherein the counterion is hydrogen and wherein R₁ and R₂ are selected from the group consisting of C₁-C₆ alkyl and C₆-C₂₀ aryl, which can be substituted with a substituent selected from the group consisting of amino, hydroxyl, or carboxyl. Such compounds include dimeric 2-[4′-(α-nitroso)isobutyrylphenyl]propionic acid.

Such compounds also include C-nitroso compounds containing a moiety of Formula (N-II):

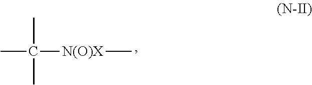

wherein X is S, O, or NR, wherein R is selected from the group consisting of C₁-C₆ alkyl which is unsubstituted or is substituted with one or more alcohol, ether, ester, or amide groups which contain from 1 to 10 carbon atoms; typically, these compounds have a molecular weight of from 100 to about 1000. A preferred subgenus of this alternative comprises the structure

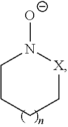

wherein X is S, O, or NR, wherein R is defined as above and n is from 0 to 4; these compounds can alternatively be protonated to remove the negative charge. The structure can be substituted with C₁-C₆ alkyl or C₁-C₆ alkylcarbonyl and can include the modification that the carbon pendant to X in the ring or a carbon within the parenthesis can also be part of another ring.

The compounds include C-nitroso derivatives of acetylsalicylic acid, C-nitroso derivatives of propranolol, C-nitroso derivatives of nadolol, C-nitroso derivatives of carvedilol, C-nitroso derivatives of prazosin, C-nitroso derivatives of tinolol, C-nitroso derivatives of metoprolol, C-nitroso derivatives of pindolol, C-nitroso derivatives of labetalol, C-nitroso derivatives of triamterene, C-nitroso derivatives of furosemide, C-nitroso derivatives of enalapril, C-nitroso derivatives of ramipril, C-nitroso derivatives of lovastatin, C-nitroso derivatives of pravastatin, C-nitroso derivatives of gemfibrozil, C-nitroso derivatives of clofibrate, C-nitroso derivatives of nifedipine, C-nitroso derivatives of amlodipine, C-nitroso derivatives of diltiazem, C-nitroso derivatives of verapamil, C-nitroso derivatives of cimetidine, C-nitroso derivatives of ranitidine, C-nitroso derivatives of albuterol, C-nitroso derivatives of ipratropium bromide, C-nitroso derivatives of memantine, C-nitroso derivatives of 10-deacetylbaccatin III, C-nitroso derivatives of taxol, C-nitroso derivatives of pretomanid, C-nitroso derivatives of dalcetrapib, C-nitroso derivatives of superoxide dismutase mimetics, C-nitroso derivatives of allopurinol, C-nitroso derivatives of celecoxib, C-nitroso derivatives of indomethacin, C-nitroso derivatives of thioflosulide, and C-nitroso derivatives of etodolac.

Inositol hexaphosphate (IP6) is described as an arrestin sequestrant (Y. K. Peterson & L. M. Luttrell, “The Diverse Roles of Arrestin Scaffolds in G Protein-Coupled Receptor Signaling,” Pharmacol. Rev. 69: 256-297 (2017)).

Additionally, it is known that second messenger-stimulated kinases also play a role in the desensitization of GPCR-coupled receptors. For Gs-coupled receptors, which include β₁-, β₂-, and β₃-adrenergic receptors, the relevant second messenger-stimulated kinase is cAMP-dependent protein kinase, also known as protein kinase A (R. J. Lefkowitz, “G Protein Coupled Receptors III. New Roles for Receptor Kinases and β-Arrestins in Receptor Signaling and Desensitization,” J. Biol. Chem. 273: 18677-18680 (1998)). In this context, phosphorylation occurs on serine residues located in the third cytoplasmic loop or carboxyl-terminal tail of the receptors. Phosphorylation directly alters receptor conformation so that interaction of the receptor with the corresponding G protein is impaired. This type of receptor regulation generally mediates a type of desensitization referred to as heterologous or non-agonist-specific desensitization because any stimulant that elevates cAMP has the potential to cause the phosphorylation and resulting desensitization of any GPCR containing an appropriate protein kinase A (PKA) consensus phosphorylation site.

The major cellular mechanism mediating rapid, agonist-specific, or homologous desensitization of G protein-coupled receptors consists of a two-step process in which the agonist-occupied receptors are phosphorylated by a GRK and then bind an arrestin protein, which sterically interdicts signaling to the G protein. GRK-catalyzed activity may be allosteric; other factors regulating activity of GRKs include protein kinase C, lipids, and calcium-binding proteins such as recoverin or calmodulin. GRK phosphorylation of GPCRs potentiates the binding of arrestins and the internalization and sequestration of these receptors. Therefore, as stated below, any agents that can interfere with any aspect of this process may be useful for inhibiting the activity of arrestins and thus prevent desensitization of the β-adrenergic receptors.

One alternative of an agent that can inhibit the activity of arrestins is an inhibitor of the arrestin itself. Inhibitors of arrestins include barbadin.

Another alternative of an agent that can inhibit the activity of arrestins is an inhibitor of GRKs. Inhibitors of GRKs are disclosed in United States Patent Application Publication No. 2004/0053852 by Stamler et al. GRKs phosphorylate agonist-occupied GPCRs, thereby promoting binding by the β-arrestin molecules to the phosphorylated GPCRs. Therefore, agents that inhibit the activity of GRKs can also block the activity of β-arrestin. These agents include NO donors that donate nitric oxide or a related species, including S-nitroso, O-nitroso, C-nitroso, and N-nitroso compounds. These compounds include, but are not limited to, S-nitrosoglutathione (GSNO), S-nitroso-N-acetylpenicillamine, S-nitroso-cysteine and ethyl ester thereof, S-nitroso-cysteinyl glycine, S-nitroso-γ-methyl-L-homocysteine, S-nitroso-L-homocysteine, S-nitroso-γ-thio-L-leucine, S-nitroso-δ-thio-L-leucine, and S-nitrosoalbumin. Examples of other NO donors useful herein are sodium nitroprusside (nipride), ethyl nitrite, nitroglycerin, SIN1(molsidomine), furoxamines, and N-hydroxy-(N-nitrosamine).

Another agent that can be used to block the activity of arrestin is the arrestin sequestrant IP6 (inositol hexakisphosphate) (Y. K. Peterson & L. M. Luttrell, “The Diverse Roles of Arrestin Scaffolds in G Protein-Coupled Receptor Signaling,” Pharmacol. Rev. 69: 256-297 (2017)).

Additionally, according to R. J. Lefkowitz, “G Protein Coupled Receptors III. New Roles for Receptor Kinases and β-Arrestins in Receptor Signaling and Desensitization,” J. Biol. Chem. 273: 18677-18680 (1998), protein kinase A inhibitors may also be useful in blocking the activity of arrestin.

Protein kinase A inhibitors include the following agents: (1) H89 (N-[2-[[3-(4-bromophenyl)-2-propenyl]amino]ethyl]-5-isoquinolinesulfonamide dihydrochloride) (J. Leemhuis et al., “The Protein Kinase A Inhibitor H89 Acts on Cell Morphology by Inhibiting Rho Kinase,” J. Pharmacol. Exp. Ther. 300: 1000-1007 (2002); (2) N-(ω-undecylenoyl)phenylalanine (U.S. Pat. No. 7,871,635 to Stolz et al.); (3) 3′,5′-cyclic monophosphorothioate-R, H-7 (5-(2-methylpiperazin-1-yl)sulfonylisoquinoline dihydrochloride), H-8 (N-[2-(methylamino)ethyl]-5-isoquinolinesulfonamide dihydrochloride), and H-9 (N-(2-aminoethyl)-5-isoquinolinesulfonamide) (U.S. Pat. No. 9,744,332 to Berggren et al.); (4) 6-22 amide, a peptide with the sequence Thr-Tyr-Ala-Asp-Phe-Ile-Ala-Ser-Gly-Arg-Thr-Gly-Arg-Arg-Asn-Ala-Ile-NH₂ (SEQ ID NO: 2) (U.S. Pat. No. 10,485,845 to Ambron et al.); protein kinase A inhibitors including fasudil, N-[2-(phosphorylated bromonitroarginylamino)ethyl]-5-isoquinoline sulfonamide, 1-(5-quinolinesulfonyl)piperazine, 4-cyano-3-methylisoquinoline, acetamido-4-cyano-3-methylisoquinoline, 8-bromo-2-monoacyladenosine-3,5-cyclic monophosphorothioate, adenosine 3,5-cyclic monophosphorothioate, 2-O-monobutyl-cyclic adenosine monophosphate, 8-chloro-cyclic adenosine monophosphate, N-[2-(cinnamoylamino acid)]-5-isoquinolinone, reverse phase-8-hexylamino adenosine 3,5-monophosphorothioate, reverse phase-8-piperidinyladenosine-cyclic adenosine monophosphate, reverse phase-adenosine 3,5-cyclic monophosphorothioate, 5-iodotuberculin, 8-hydroxyadenosine-3,5-monophosphorothioate, calphostin C, daphnetin, reverse phase-8-chlorophenyl-cyclic adenosine monophosphate, reverse phase-cyclic adenosine monophosphate, reverse phase-8-Br-cyclic adenosine monophosphate, 1-(5-isoquinolinesulfonyl)-2-methylpiperidine, 8-hydroxyadenosine-3′,5′-monophosphate, 8-hexylaminoadenosine-3′,5′-monophosphate, and reverse phase-adenosine 3′,5′-cyclic monophosphate (United States Patent Application Publication No. 2019/0343861 by Dai); erbstatin (United States Patent Application Publication No. 2008/0138834 by Emans et al.); protein kinase A inhibitors including adenosine 3′,5′-cyclic phosphorothiolate, 8-bromo-adenosine 3′,5′-cyclic monophosphorothioate, 4-cyano-3-methylisoquinoline, 1-(5-isoquinolinesulfonyl)-2-methylpiperazine, N-[2-(methylamino)ethyl]-5-isoquinolinesulfonamide, isoquinolinesulfonamide, N-2-aminoethyl)-5-isoquinolinesulfonamide, N-[2-((p-bromocinnamyl)amino)ethyl]-5-isoquinolinesulfonamide and (5-isoquinolinesulfonyl)piperazine (United States Patent Application Publication No. 2006/0099568 by Jang et al.); KT 5720 ((9R,10S,12S)-2,3,9,10,11,12-hexahydro-10-hydroxy-9-methyl-1-oxo-9,12-epoxy-1H-diindolo[1,2,3-fg:3′,2′,1′-k/]pyrrolo[3,4-i][1,6]benzodiazocine-10-carboxylic acid hexyl ester); myristoylated PKI 14-22 amide (Myr-Gly-Arg-Thr-Gly-Arg-Arg-Asn-Ala-Ile-NH₂); cAMPs-Rp triethylammonium salt ((R)-adenosine, cyclic 3′,5′-(hydrogenphosphorothioate) triethylammonium); daphnetin; HA-100 (5-(1-piperazinylsulfonyl)isoquinoline dihydrochloride); and HA-100 (5-(1-piperazinylsulfonyl)isoquinoline dihydrochloride).

In addition, as shown in Example 5, the enzyme phospholipase C plays a key role in the pathway leading to asthmatic symptoms, as it cleaves a phosphodiester bond in membrane phospholipids, resulting in the formation of a 1,2-diglyceride. Arachidonate is then released from the diglyceride by the sequential actions of diglyceride lipase and monoglyceride lipase. Once released, a portion of the arachidonate is metabolized rapidly, leading to oxygenated products, including eicosanoids such as prostaglandins. Thus, any treatment that can inhibit phospholipase C activity is relevant for the treatment of asthma. Similarly, inhibitors of phospholipase C are also relevant for the treatment of other respiratory diseases and conditions such as chronic obstructive pulmonary disease and other diseases and conditions that can be treated by methods and compositions according to the present invention.

Therefore, in yet another alternative according to the present invention, the nadolol, the derivative or analog of nadolol, or the prodrug of the nadolol or the derivative or analog of the nadolol is administered together with a therapeutically effective quantity of a phospholipase C inhibitor.

Phospholipase C inhibitors include, but are not limited to: sodium aristolochate; D609 (sodium tricyclodecan-9-yl xanthogenate); D-erythro-dihydrosphingosine; U-73122 (1-(6-((17β-3-methoxyestra-1,3,5(10)-trien-17-yl)amino)hexyl)-1H-pyrrole-2,5-dione); pyrrolidinethiocarbamate; neomycin sulfate; thielavin B; edelfosine; compounds described in U.S. Pat. No. 7,262,197 to Lagu et al., United States Patent Application Publication No. 2004/0242639 by Lagu et al., United States Patent Application Publication No. 2004/0235855 by Lagu et al., and United States Patent Application Publication No. 2004/0235827 by Lagu et al., including heterocyclyl-substituted anilino compounds, including: N-[2-[4-(diphenylmethyl)-1-piperazinyl]-5-(1-piperazinylcarbonyl)phenyl]-4-methyl-benzamide; 5-(4-chlorophenyl)-N-[2-[4-(diphenylmethyl)-1-piperazinyl]-5-(1-piperazinylcarbonyl)phenyl]-2-methyl-3-furancarboxamide; N-[2-[4-(diphenylmethyl)-1-piperazinyl]-5-(1-piperazinylcarbonyl)phenyl]-2-furancarboxamide; N-[2-[4-(diphenylmethyl)-1-piperazinyl]-5-(1-piperazinylcarbonyl)phenyl]-propanamide; 3-[[(phenylamino)carbonyl]amino]-4-[4-(phenylmethyl)-1-piperidinyl]-benzamide; 3-[[(phenylamino)carbonyl]amino]-4-(4-phenyl-1-piperidinyl)-benzamide; 3-[[(1,3-benzodioxol-5-ylamino)carbonyl]amino]-4-(4-phenyl-1-piperidinyl)-benzamide; N-[2-(4,4-diphenyl-1-piperidinyl)-5-(1-piperazinylcarbonyl)phenyl]-N′-phenyl-urea; N-[5-(aminocarbonyl)-2-[4-(phenylmethyl)-1-piperidinyl]phenyl]hydrazine-carboxamide; 4-[4-(diphenylmethyl)-1-piperidinyl]-3-[[(phenylamino)carbonyl]amino]-benzamide; 4-[4-(diphenylmethylene)-1-piperidinyl]-3-[[(phenylamino)carbonyl]amino]-benzamide; N-[2-[4-(diphenylmethyl)-1-piperidinyl]-4-(1-piperazinylcarbonyl)phenyl]-N′-phenyl-urea; N-cyclohexyl-N-[2-[4-(diphenylmethyl)-1-piperidinyl]-4-(1-piperazinylcarbonyl) phenyl]-urea; 4-[4-(2-methoxyphenyl)-1-piperazinyl]-3-[[(phenylamino)carbonyl]amino]-benzamide; 3-[[(phenylamino)carbonyl]amino]-4-[4-(phenylmethyl)-1-piperazinyl]-benzamide; 4-[4-[bis(4-fluorophenyl)methyl]-1-piperazinyl]-3-[[(phenylamino)carbonyl]amino]-benzamide; 4-[4-[bis(4-fluorophenyl)methyl]-1-piperazinyl]-3-[[[(2-fluorophenyl)amino]carbonyl]amino]-benzamide; 4-[4-(diphenylmethyl)-1-piperazinyl]-3-[[[(4-nitrophenyl)amino]carbonyl]amino]-benzamide; 4-[4-(diphenylmethyl)-1-piperazinyl]-3-[[[(phenylmethyl)amino]carbonyl]amino]-benzamide, 3-[[[(3,5-dimethylphenyl)amino]carbonyl]amino]-4-[4-(diphenylmethyl)-1-piperazinyl]-benzamide; 4-[4-(diphenylmethyl)-1-piperazinyl]-3-[[(phenylamino)carbonyl]amino]-benzamide; 4-[4-(9H-fluoren-9-yl)-1-piperazinyl]-3-[[(phenylamino)carbonyl]amino]-benzamide; 3-[[(cyclohexylamino)carbonyl]amino]-4-[4-(diphenylmethyl)-1-piperazinyl]-benzamide; 4-[4-(diphenylmethyl)-1-piperazinyl]-3-[[[[(1S)-1-phenylethyl]amino]carbonyl]amino]-benzamide; 3-[[(butylamino)carbonyl]amino]-4-[4-(diphenylmethyl)-1-piperazinyl]-benzamide; 4-[4-(diphenylmethyl)-1-piperazinyl]-3-[[[(4-fluorophenyl)amino]carbonyl]amino]-benzamide; 3-[[(1,3-benzodioxol-5-ylamino)carbonyl]amino]-4-[4-[bis-(4-fluorophenyl)methyl]-1-piperazinyl]-benzamide; 4-[4-[bis(4-fluorophenyl)methyl]-1-piperazinyl]-3-[[[(2,4-dimethylphenyl)amino]carbonyl]amino]-benzamide; 4-[4-[bis(4-fluorophenyl)methyl]-1-piperazinyl]-3-[[[(1-phenylethyl)amino]carbonyl]amino]-benzamide; 4-[4-[bis(4-fluorophenyl)methyl]-1-piperazinyl]-3-[[[(2-methoxyphenyl)amino]carbonyl]amino]-benzamide; 4-[4-[bis(4-fluorophenyl)methyl]-1-piperazinyl]-3-[[[(2,4-dimethoxyphenyl)amino]carbonyl]amino]-benzamide; 4-[4-[bis(4-fluorophenyl)methyl]-1-piperazinyl]-3-[[[[4-(dimethylamino)phenyl]amino]carbonyl]amino]-benzamide; 4-[4-[bis(4-fluorophenyl)methyl]-1-piperazinyl]-3-[[[(4-methoxyphenyl)amino]carbonyl]amino]-benzamide; 4-[4-[bis(4-fluorophenyl)methyl]-1-piperazinyl]-3-[[[(phenylmethyl)amino]thioxomethyl]amino]-benzamide; 4-[4-[bis(4-fluorophenyl)methyl]-1-piperazinyl]-3-[[(phenylamino)thioxomethyl]amino]-benzamide; N-[2-[4-(diphenylmethyl)-1-piperazinyl]-5-[(4-methyl-1-piperazinyl)carbonyl]phenyl]-N′-phenylurea; N-[2-[4-(diphenylmethyl)-1-piperazinyl]-5-[(hexahydro-1H-1,4-diazepin-1-yl)carbonyl]phenyl]-N′-phenylurea, N-cyclohexyl-N′-[2-[4-(diphenylmethyl)-1-piperazinyl]-5-[(hexahydro-1H-1,4-diazepin-1-yl)carbonyl]-phenyl]urea; N-(2-aminoethyl)-4-[4-(diphenylmethyl)-1-piperazinyl]-3-[[(phenylamino)carbonyl]amino]-benzamide; and N-(2-aminoethyl)-3-[[(cyclohexylamino)carbonyl]amino]-4-[4-(diphenylmethyl)-1-piperazinyl]-benzamide; N-[2-[4-(diphenylmethyl)-1-piperazinyl]-5-(1-piperazinylcarbonyl)phenyl]-4-methyl-benzamide; 5-(4-chlorophenyl)-N-[2-[4-(diphenylmethyl)-1-piperazinyl]-5-(1-piperazinylcarbonyl)phenyl]-2-methyl-3-furancarboxamide; N-[2-[4-(diphenylmethyl)-1-piperazinyl]-5-(1-piperazinylcarbonyl)phenyl]-2-furancarboxamide; N-[2-[4-(diphenylmethyl)-1-piperazinyl]-5-(1-piperazinylcarbonyl)phenyl]-propanamide; N-[2-[4-(diphenylmethyl)-1-piperazinyl]-5-(1-piperazinylcarbonyl)phenyl]-4-methyl-benzenesulfonamide; 4-chloro-N-[2-[4-(diphenylmethyl)-1-piperazinyl]-5-(1-piperazinylcarbonyl)phenyl]-benzenesulfonamide; N-[2-[4-(diphenylmethyl)-1-piperazinyl]-5-(1-piperazinylcarbonyl)phenyl]-1-butanesulfonamide; N-[2-[4-(diphenylmethyl)-1-piperazinyl]-5-(1-piperazinylcarbonyl)phenyl]-methanesulfonamide; N-[2-[4-(diphenylmethyl)-1-piperazinyl]-5-(methylsulfonyl)phenyl]-N-phenyl-urea; N-[2-[4-[bis(4-fluorophenyl)methyl]-1-piperazinyl]-5-(hydroxymethyl)phenyl]-N-phenyl-urea; and 4-[4-[bis(4-fluorophenyl)methyl]-1-piperazin-yl]-3-[[(phenylamino)carbonyl]amino]-benzoic acid methyl ester; DCIC (3,4-dichloroisocoumarin); and calporoside or calporoside derivatives, including compounds disclosed in U.S. Pat. No. 6,596,984 to Eder et al., including compounds of Formula (C-I):

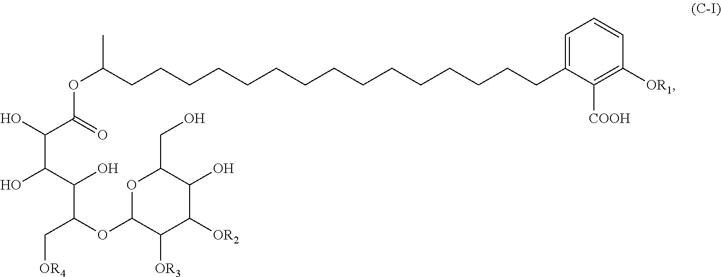

wherein:
(1) R₁, R₂, and R₃ are independently selected from the group consisting of H and C₁-C₁₀ acyl; and
(2) R₄ is H or —C(O)(CH₂)ₙCOOH, wherein n is from 1 to 7, with the proviso that R₁, R₂, R₃, and R₄ are not all H;
   and RHC-80267 (1,6-bis-(cyclohexyloximinocarbonylamino)-hexane).

In methods and compositions according to the present invention, for small molecules other than nadolol or derivatives or analogs of nadolol as described above useful in combination with the β-adrenergic inverse agonist can be optionally substituted with one or more groups that do not substantially affect the pharmacological activity of the small molecule. Definitions for a number of common groups that can be used as optional substituents have been provided above; however, the omission of any group from these definitions cannot be taken to mean that such a group cannot be used as an optional substituent as long as the chemical and pharmacological requirements for an optional substituent are satisfied.

As used herein, the term “alkyl” refers to an unbranched, branched, or cyclic saturated hydrocarbyl residue, or a combination thereof, of from 1 to 12 carbon atoms that can be optionally substituted; the alkyl residues contain only C and H when unsubstituted. Typically, the unbranched or branched saturated hydrocarbyl residue is from 1 to 6 carbon atoms, which is referred to herein as “lower alkyl.” When the alkyl residue is cyclic and includes a ring, it is understood that the hydrocarbyl residue includes at least three carbon atoms, which is the minimum number to form a ring. As used herein, the term “alkenyl” refers to an unbranched, branched or cyclic hydrocarbyl residue having one or more carbon-carbon double bonds. As used herein, the term “alkynyl” refers to an unbranched, branched, or cyclic hydrocarbyl residue having one or more carbon-carbon triple bonds; the residue can also include one or more double bonds. With respect to the use of “alkenyl” or “alkynyl,” the presence of multiple double bonds cannot produce an aromatic ring. As used herein, the terms “hydroxyalkyl,” “hydroxyalkenyl,” and “hydroxyalkynyl,” respectively, refer to an alkyl, alkenyl, or alkynyl group including one or more hydroxyl groups as substituents; as detailed below, further substituents can be optionally included.

Substituent groups useful for substituting saturated carbon atoms in the specified group, moiety, or radical include, but are not limited to, —Z^{a}, ═O, —OZ^{b}, —SZ^{b}, ═S⁻, —NZ^{c}Z^{c}, ═NZ^{b}, ═N—OZ^{b}, trihalomethyl, —CF₃, —CN, —OCN, —SCN, —NO, —NO₂, ═N₂, —N₃, —S(O)₂Z^{b}, —S(O)₂NZ^{b}, —S(O₂)O⁻, —S(O₂)OZ^{b}, —OS(O₂)OZ^{b}, —OS(O₂)O⁻, —OS(O₂)OZ^{b}, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)Z^{b}, —NZ^{b}C(S)Z^{b}, —NZ^{b}C(O)O⁻, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, —NZ^{b}C(NZ^{b})NZ^{c}Z^{c}, wherein Z^{a} is selected from the group consisting of alkyl, cycloalkyl, heteroalkyl, cycloheteroalkyl, aryl, arylalkyl, heteroaryl and heteroarylalkyl; each Z^{b} is independently hydrogen or Z^{a}; and each Z^{c} is independently Z^{b} or, alternatively, the two Z° 's may be taken together with the nitrogen atom to which they are bonded to form a 4-, 5-, 6-, or 7-membered cycloheteroalkyl ring structure which may optionally include from 1 to 4 of the same or different heteroatoms selected from the group consisting of N, O, and S. As specific examples, —NZ^{c}Z^{c} is meant to include —NH₂, —NH-alkyl, —N-pyrrolidinyl, and —N-morpholinyl, but is not limited to those specific alternatives and includes other alternatives known in the art. Similarly, as another specific example, a substituted alkyl is meant to include -alkylene-O-alkyl, -alkylene-heteroaryl, -alkylene-cycloheteroaryl, -alkylene-C(O)OZ^{b}, -alkylene-C(O)NZ^{b}Z^{b}, and —CH₂—CH₂—C(O)—CH₃, but is not limited to those specific alternatives and includes other alternatives known in the art. The one or more substituent groups, together with the atoms to which they are bonded, may form a cyclic ring, including, but not limited to, cycloalkyl and cycloheteroalkyl.

Similarly, substituent groups useful for substituting unsaturated carbon atoms in the specified group, moiety, or radical include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —N₃, —S(O)₂Z^{b}, —S(O₂)O⁻, —S(O₂)OZ^{b}, —OS(O₂)OZ^{b}, —OS(O₂)O—, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O—, —OC(O)OZ^{b}, —OC(S)OZ^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z° are as defined above.

Similarly, substituent groups useful for substituting nitrogen atoms in heteroalkyl and cycloheteroalkyl groups include, but are not limited to, —Z^{a}, halo, —O—, —OZ^{b}, —SZ^{b}, —S⁻, —NZ^{c}Z^{c}, trihalomethyl, —CF₃, —CN, —OCN, —SCN, —NO, —NO₂, —S(O)₂Z^{b}, —S(O₂)O⁻, —S(O₂)OZ^{b}, —OS(O₂)OZ^{b}, —OS(O₂)O⁻, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)OZ^{b}, —OC(S)OZ^{b}, —NZ^{b}C(O)Z^{b}, —NZ^{b}C(S)Z^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z° are as defined above.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms (such as the enantiomerically pure isomers, the E and Z isomers, and other stereoisomeric forms) as well as mixtures of stereoisomers in varying degrees of chiral purity or percentage of E and Z, including racemic mixtures, mixtures of diastereomers, and mixtures of E and Z isomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. Such separation techniques include, but are not limited to, chiral high pressure liquid chromatography (HPLC) and formation and crystallization of chiral salts. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted olefin isomer. When the chemical name does not specify the isomeric form of the compound, it denotes any one of the possible isomeric forms or mixtures of those isomeric forms of the compound.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein includes two or more interconvertible compounds resulting from at least one formal migration of a hydrogen atom and at least one change in valency (e.g., a single bond to a double bond, a triple bond to a double bond, a triple bond to a single bond, or vice versa). The exact ratio of the tautomers present can depend on several factors, including pH, solvent, and temperature. Tautomerization reactions can be catalyzed by acid or base. Examples of tautomerization include keto/enol, amide/imide, lactam/lactim, and enamine/imine.

In addition to the substituents described above, alkyl, alkenyl and alkynyl groups can alternatively or in addition be substituted by C₁-C₈ acyl, C₂-C₈ heteroacyl, C₆-C₁₀ aryl, C₃-C₈ cycloalkyl, C₃-C₈ heterocyclyl, or C₅-C₁ heteroaryl, each of which can be optionally substituted. Also, in addition, when two groups capable of forming a ring having 5 to 8 ring members are present on the same or adjacent atoms, the two groups can optionally be taken together with the atom or atoms in the substituent groups to which they are attached to form such a ring.

“Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group.

Similarly, “heterocyclyl” may be used to describe a non-aromatic cyclic group that contains at least one heteroatom (typically selected from N, O and S) as a ring member and that is connected to the molecule via a ring atom, which may be C (carbon-linked) or N (nitrogen-linked); and “heterocyclylalkyl” may be used to describe such a group that is connected to another molecule through a linker. The heterocyclyl can be fully saturated or partially saturated, but non-aromatic. The sizes and substituents that are suitable for the cycloalkyl, cycloalkylalkyl, heterocyclyl, and heterocyclylalkyl groups are the same as those described above for alkyl groups. The heterocyclyl groups typically contain 1, 2 or 3 heteroatoms, selected from N, O and S as ring members; and the N or S can be substituted with the groups commonly found on these atoms in heterocyclic systems. As used herein, these terms also include rings that contain a double bond or two, as long as the ring that is attached is not aromatic. The substituted cycloalkyl and heterocyclyl groups also include cycloalkyl or heterocyclic rings fused to an aromatic ring or heteroaromatic ring, provided the point of attachment of the group is to the cycloalkyl or heterocyclyl ring rather than to the aromatic/heteroaromatic ring.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C₇-arylalkyl group, and phenylethyl is a C₈-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C₇-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. The general term “alkylene” encompasses more specific examples such as “ethylene,” wherein n is 2, “propylene,” wherein n is 3, and “butylene,” wherein n is 4. The hydrocarbyl groups of the alkylene can be optionally substituted as described above.

In general, any alkyl, alkenyl, alkynyl, acyl, or aryl or arylalkyl group that is contained in a substituent may itself optionally be substituted by additional substituents. The nature of these substituents is similar to those recited with regard to the primary substituents themselves if the substituents are not otherwise described.

“Amino” as used herein refers to —NH₂, but where an amino is described as “substituted” or “optionally substituted,” the term includes NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

Other combinations of substituents are known in the art and are described, for example, in U.S. Pat. No. 8,344,162 to Jung et al., incorporated herein by this reference. For example, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure.

Accordingly, methods and compositions according to the present invention encompass analogs and derivatives of small molecules, other than nadolol or the analogs or derivatives of nadolol described above, that are optionally substituted, provided that the optionally substituted small molecules possess substantially equivalent pharmacological activity to the unsubstituted small molecules as defined in terms of their activity. The activity can be assayed by methods known in the art, including enzyme assays, in vivo assays on airway hyperresponsiveness, assays of the effect of the optionally substituted small molecules on arrestin-2 concentration or activity, assays determining the effect of the optionally substituted small molecules on the activity of β₂-adrenergic receptors, and other methods known in the art. Such optionally substituted small molecules include, but are not necessarily limited to, molecules in which the substitutions are considered to be bioisosteric. Bioisosterism is a well-known tool for predicting the biological activity of compounds, based on the premise that compounds with similar size, shape, and electron density can have similar biological activity. To form a bioisostere of a given molecule, one can replace one or more atoms or groups in the original molecule with known bioisosteric replacements for that atom or group. Known bioisosteric replacements include, but are not necessarily limited to, the interchangeability of —F, —OH, —NH₂, —Cl, and —CH₃, the interchangeability of —Br and -i-C₃H₇; the interchangeability of —I and -t-C₄H₉; the interchangeability of —O—, —S—, —NH₂—, —CH₂—, and —Se—; the interchangeability of —N═, —CH═, and —P═ in cyclic or noncyclic moieties; the interchangeability of phenyl and pyridyl groups; the interchangeability of —C═C— and —S— (for example, benzene and thiophene); the interchangeability of an aromatic nitrogen (R₁—N(R₃)—R₂) for an unsaturated carbon ((R₁—C(═R₃)—R₂); and the interchangeability of —CO—, —SO—, and —SO₂—. Other alternatives for bioisosteric replacements are known in the art.

When a therapeutically active compound employed in methods or compositions according to the present application is a protein, protein fragment, polypeptide, or peptide, the protein, protein fragment, polypeptide, or peptide can be modified by the inclusion of one or more conservative amino acid substitutions, as long such conservative amino acid substitutions substantially preserve the biological activity of the therapeutically active compound. More specifically, in a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al., Molecular Biology of the Gene, 4th Edition, 1987, Benjamin/Cummings, p. 224). In particular, such a conservative variant has a modified amino acid sequence, such that the change(s) do not substantially alter the protein's (the conservative variant's) secondary or tertiary structure and/or activity, specifically binding activity in this context. Conservative amino acid substitution generally involves substitutions of amino acids with residues having similar properties (e.g., acidic, basic, positively or negatively charged, polar or non-polar, or other similarities) such that the substitutions of even critical amino acids do not substantially alter structure and/or activity. Conservative substitution tables providing functionally similar amino acids are well known in the art. For example, one exemplary guideline to select conservative substitutions includes (original residue followed by exemplary substitution): Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. An alternative exemplary guideline uses the following six groups, each containing amino acids that are conservative substitutions for one another: (1) alanine (A or Ala), serine (S or Ser), threonine (T or Thr); (2) aspartic acid (D or Asp), glutamic acid (E or Glu); (3) asparagine (N or Asn), glutamine (Q or Gln); (4) arginine (R or Arg), lysine (K or Lys); (5) isoleucine (I or Ile), leucine (L or Leu), methionine (M or Met), valine (V or Val); and (6) phenylalanine (F or Phe), tyrosine (Y or Tyr), tryptophan (W or Trp); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. As another example, for some purposes, one may regard all non-polar amino acids as conservative substitutions for each other.

Accordingly, one aspect of the present invention is a method for treatment of pulmonary airway disease in a subject suffering from pulmonary airway disease comprising administration of a therapeutically effective quantity of nadolol or a derivative or analog of nadolol to inhibit the β-arrestin pathway to treat the pulmonary airway disease.

Typically, the pulmonary airway disease is selected from the group consisting of chronic obstructive pulmonary disease (COPD), asthma, bronchiectasis, bronchitis, Churg-Strauss syndrome, pulmonary sequelae of cystic fibrosis, emphysema, allergic rhinitis, pneumonia, and pulmonary symptoms associated with infection with SARS-CoV-2. Preferably, the pulmonary airway disease is chronic obstructive pulmonary disease (COPD); the COPD can be associated with another disease or condition, such as infection with SARS-CoV-2.

Typically, the method comprises administration of a therapeutically effective quantity of nadolol. A preferable stereoisomer of nadolol is the RSR stereoisomer as described above.

Alternatively, the method comprises administration of a therapeutically effective quantity of a derivative or analog of nadolol that is a compound of Formula (I):

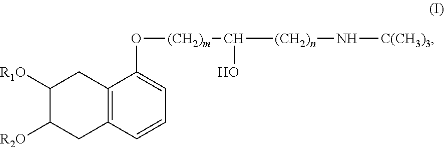

wherein R₁ is hydrogen or lower alkyl, R₂ is hydrogen or lower alkyl, and m and n are 1 to 3, with the proviso that wherein R₁ and R₂ are both hydrogen and m is 1, n is other than 1.

Typically, the method exerts a therapeutic effect that is a reduction in pulmonary airway constriction hyperresponsiveness. Typically, the method also exerts a therapeutic effect that is an upregulation of pulmonary 2-adrenergic receptors. Typically, the method also exerts a therapeutic effect that is increased pulmonary airway relaxation responsiveness to 2-adrenergic agonist drugs.

Typically, the nadolol or the derivative or analog of nadolol is administered by a route selected from the group consisting of oral, sustained-release oral, parenteral, sublingual, buccal, administration by insufflation, and administration by inhalation. Preferably, the nadolol or the derivative or analog of nadolol is administered by inhalation; this route is particularly desirable for administration of nadolol. When nadolol is administered by inhalation, such as in a composition comprising nadolol, a pharmaceutically acceptable excipient, and, in some alternatives, at least one additional therapeutically active agent, the administration may produce evanescent blood levels of nadolol or no detectable blood levels of nadolol using the method for assay that is approved for the 505(j) generic pathway (USP method).

Typically, when sustained-release oral administration is employed, the method of administration of the nadolol or the derivative or analog of nadolol results in continuous levels of the nadolol or the derivative or analog of nadolol in the bloodstream. Also, typically, the nadolol or the derivative or analog of nadolol is administered over time in a series of graduated doses starting from the lowest dose and increasing to the highest dose. In this alternative, preferably, when the highest dose is reached, the nadolol or the derivative or analog of nadolol continues to be administered at that dose.

Typically, in methods according to the present invention, the inhibition of β-arrestin prevents or reverses the desensitization of β₂-adrenergic receptors. Also, typically, in methods according to the present invention, the inhibition of β-arrestin prevents or reverses the internalization of β₂-adrenergic receptors. Additionally, typically, in methods according to the present invention, the inhibition of β-arrestin prevents or reverses phosphorylation of β₂-adrenergic receptors by a second-messenger-specific protein kinase or a specific G-protein-coupled receptor kinase. Similarly, typically, in methods according to the present invention, the inhibition of β-arrestin prevents or reverses degradation of a second messenger by a scaffolding phosphodiesterase. Typically, in methods according to the present invention, the inhibition of β-arrestin prevents or reverses the occurrence of mucous metaplasia or goblet cell hyperplasia.

In one alternative of a method according to the present invention, the method further comprises administration of a therapeutically effective quantity of at least one additional therapeutic agent.

In one alternative, the additional therapeutic agent is a β₂-selective adrenergic agonist. Typically, the β₂-selective adrenergic agonist is selected from the group consisting of albuterol, arfomoterol, bambuterol, bitolterol, broxaterol, buphenine, carbuterol, clenbuterol, clorprenaline, colterol, dobutamine, fenoterol, formoterol, isoetharine, isoprenaline, levabuterol, levosalbutamol, mabuterol, metaprotenerol, methoxyphenamine, pirbuterol, procaterol, ractopamine, reproterol, ritodrine, salmeterol, terbutaline, zilpaterol, and the salts, solvates, and prodrugs thereof.

In another alternative, the additional therapeutic agent is a corticosteroid. Typically, the corticosteroid is selected from the group consisting of AZD-5423 (2,2,2-trifluoro-N-[(1R,2S)-1-{[1-(4-fluorophenyl)-1H-indazol-5-yl]oxy}-1-(3-methoxyphenyl)-2-propanyl]acetamide), beclomethasone, budesonide, ciclesonide, deflazacort, flunisolide, fluticasone, methylprednisolone, mometasone, prednisolone, prednisone, dexamethasone, and triamcinolone, and the salts, solvates, and prodrugs thereof.

In another alternative, the additional therapeutic agent is an anticholinergic drug. Typically, the anticholinergic drug is selected from the group consisting of ipratropium bromide, tiotropium bromide, oxitropium bromide, abediterol, aclidinium bromide, glycopyrronium bromide, umeclidinium bromide, and the salts, solvates, and prodrugs thereof.

In yet another alternative, the additional therapeutic agent is a biological (B. L. Walker & R. Leigh, “Use of Biologicals as Immunotherapy in Asthma and Related Diseases,” Expert Rev. Clin. Immunol. 4: 753-756 (2008)). Typically, the biological biological is selected from the group consisting of an anti-IL4 antibody, an anti-IL13 antibody, an inhibitor of both IL4 and IL13, an anti-IL5 antibody, and an anti-IL8 antibody. Anti-IL4 antibodies include, but are not limited to, a humanized anti-IL4 monoclonal antibody, pasolizumab. Anti-IL13 antibodies include, but are not limited to, a human anti-IL13 monoclonal antibody, CAT-354. An inhibitor of both IL4 and IL13 is the monoclonal antibody dupilumab, which is a monoclonal antibody that binds to the a subunit of the interleukin-4 receptor (IL4Ra), which modulates the signaling of both the IL4 and the IL13 pathways; it therefore acts as a receptor antagonist (A. L. Kau & P. E. Korenblat, “Anti-Interleukin 4 and 13 for Asthma Treatment in the Era of Endotypes,” Curr. Opin. Allergy Clin. Immunol. 14: 570-575 (2014)). Anti-IL5 antibodies include, but are not limited to, the monoclonal antibodies benralizumab, mepolizumab, and reslizumab. Anti-IL8 antibodies include, but are not limited to, the human monoclonal antibody BMS-986253.

In yet another alternative, the additional therapeutic agent is a xanthine compound. Typically, the xanthine compound is selected from the group consisting of theophylline, extended-release theophylline, aminophylline, theobromine, enprofylline, diprophylline, isbufylline, choline theophyllinate, albifylline, arofylline, bamifylline, caffeine, 8-chlorotheophylline, diprophylline, doxofylline, enprofylline, etamiphylline, furafylline, 1-isobutyl-1-methylxanthine, proxyphylline, and xanthinol, and the salts, solvates, and prodrugs thereof.

In still another alternative, the additional therapeutic agent is an anti-IgE antibody. Typically, the anti-IgE antibody is a monoclonal antibody or a genetically engineered antibody that is derived from a monoclonal antibody. Typically, the anti-IgE antibody is humanized. A suitable anti-IgE antibody is omalizumab.

In still another alternative, the additional therapeutic agent is a leukotriene antagonist. Typically, the leukotriene antagonist is selected from the group consisting of montelukast, pranlukast, and zafirlukast, and the salts, solvates, and prodrugs thereof.

In still another alternative, the additional therapeutic agent is a phosphodiesterase IV inhibitor. Typically, the phosphodiesterase IV inhibitor is selected from the group consisting of roflumilast, cilomilast, piclamilast, and ibudilast, and the salts, solvates, and prodrugs thereof.

In yet another alternative, the additional therapeutic agent is a 5-lipoxygenase inhibitor. Typically, the 5-lipoxygenase inhibitor is selected from the group consisting of zileuton and fenleuton, and the salts, solvates, and prodrugs thereof.

In still another alternative, the additional therapeutic agent is a mast cell stabilizer. Typically, the mast cell stabilizer is selected from the group consisting of azelastine, cromoglicic acid, ketotifen, lodoxamide, nedocromil, olopatadine, and pemirolast, and the salts, solvates, and prodrugs thereof.

In yet another alternative, the additional therapeutic agent is an arrestin-2 inhibitor.

Suitable arrestin-2 inhibitors include, but are not limited to: a protein fragment of arrestin-2; compounds of Formula (A-I):

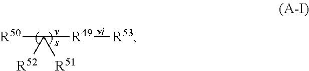

wherein:
(1) v and vi designate the particular bonds indicated in Formula (A-I);
(2) R⁴⁹ is selected from Formulas (A-I(a)), (A-I(b)), (A-I(c)), and (A-I(d)):

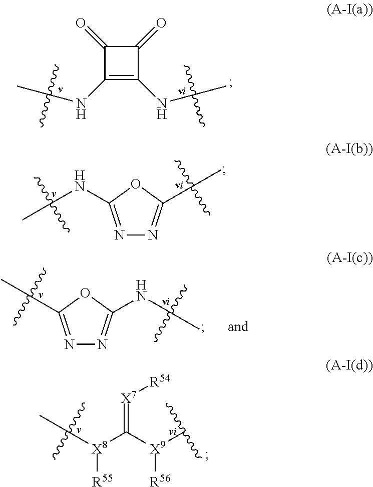

wherein X⁷, X⁸, and X⁹ are each independently O, N, or S;
(2) R⁵⁴, R⁵⁵, and R⁵⁶ are each independently H, cyano, amino, or a substituted or unsubstituted alkyl, alkanoyl, alkanoyloxy, or aryl group when X⁷, X⁸, or X⁹ are respectively N and are absent when X⁷, X⁸, or X⁹ are respectively O or S;
(3) R⁵⁰ is a substituted or unsubstituted aryl or heteroaryl group;
(4) R⁵¹ and R⁵² are each independently H or a substituted or unsubstituted alkyl group, or R⁵¹ and R⁵² together form a 3- or 4-membered cycloalkyl ring;
(5) R⁵³ is a substituted aryl group where one and only one of the substituents is a moiety of Formula (A-I(e)):

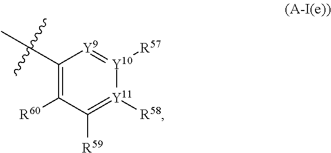

(i) wherein one of R⁵⁷ or R⁵⁸ is a moiety of Formula (A-I(f)): (A-I(f)),

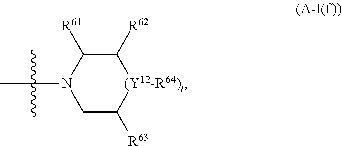

and the other is H, azido, trifluoromethyldiazirido, isocyano, isothiocyano, pentafluorosulfanyl, or a substituted or unsubstituted alkyl, alkanoyl, alkanoyloxy, aryloyl, or aryloyloxy group;
(ii) R⁵⁹ and R⁶⁰ are each independently H, halo, azido, trifluoromethyldiazirido, isocyano, isothiocyano, pentafluorosulfuryl, or a substituted or unsubstituted alkyl, alkanoyl, alkanoyloxy, aryloyl, or aryloyloxy group;
   (iii) Y⁹ is CH;
   (iv) Y¹⁰ and Y¹¹ are each independently C or N, provided that when Y¹⁰ or Y¹¹ is N then R⁵⁷ or R⁵⁸ respectively is absent;
   (v) Y¹² is CH, N, O, S, S(O), or S(O)₂;
   (vi) R⁶¹, R⁶², and R⁶³ are each independently H, azido, trifluoromethyldiazirido, isocyano, isothiocyano, or a substituted or unsubstituted alkyl group;
   (vii) R⁶⁴ is H or a substituted or unsubstituted alkyl group when Y¹² is CH or N and is absent when Y¹² is O, S, S(O), or S(O)₂; and
   (viii) t is 0 or 1; and
(6) s is 0 or 1;
   an omega-3 fatty acid that can be selected from the group consisting of DHA (docosahexaenoic acid) and EPA (eicosapentaenoic acid); a CXCR2 inhibitor that can be selected from the group consisting of SB225002 (N-(2-bromophenyl)-N′-(2-hydroxy-4-nitrophenyl)urea), AZD5069 (N-(2-((2,3-difluorobenzyl)thio)-6-(((2R,3S)-3,4-dihydroxybutan-2-yl)oxy)pyrimidin-4-yl)azetidine-1-sulfonamide); SB265610 (1-(2-bromophenyl)-3-(4-cyano-1H-benzo[d][1,2,3]triazol-7-yl)urea); navarixin; danirixin; CXCR2-IN-1 (1-(2-chloro-3-fluorophenyl)-3-[4-chloro-2-hydroxy-3-(1-methylpiperidin-4-yl)sulfonylphenyl]urea); SRT3109 (N-(2-((2,3-difluorobenzyl)thio)-6-((3,4-dihydroxybutan-2-yl)amino)pyrimidin-4-yl)azetidine-1-sulfonamide); and SRT3190 (N-[2-[(2,3-difluorophenyl)methylsulfanyl]-6-[[(2S,3R)-3,4-dihydroxybutan-2-yl]amino]pyrimidin-4-yl]azetidine-1-sulfonamide); a MyD88 inhibitor that can be selected from the group consisting of ST2825 ((4R,7R,8aR)-1′-[2-[4-[[2-(2,4-dichlorophenoxy)acetyl]amino]phenyl]acetyl]-6-oxospiro[3,4,8,8a-tetrahydro-2H-pyrrolo[2,1-b][1,3]thiazine-7,2′-pyrrolidine]-4-carboxamide) and T6167923 (4-(3-bromophenyl)sulfonyl-N-(1-thiophen-2-ylethyl)piperazine-1-carboxamide); a MD2 inhibitor that can be L48H37 ((3E,5E)-1-ethyl-3,5-bis[(2,3,4-trimethoxyphenyl)methylidene]piperidin-4-one); inositol hexaphosphate (IP6); barbadin; an inhibitor of protein kinase A that can be selected from the group consisting of: (i) H89 (N-[2-[[3-(4-bromophenyl)-2-propenyl]amino]ethyl]-5-isoquinolinesulfonamide dihydrochloride); (ii) N-(ω-undecylenoyl)phenylalanine; (iii) 3′,5′-cyclic monophosphorothioate-R; (iv) H-7 (5-(2-methylpiperazin-1-yl)sulfonylisoquinoline dihydrochloride); (v) H-9 (N-(2-aminoethyl)-5-isoquinolinesulfonamide; (vi) 6-22 amide; (vii) a protein kinase A inhibitor selected from the group consisting of: fasudil; N-[2-(phosphorylated bromonitroarginylamino)ethyl]-5-isoquinoline sulfonamide; 1-(5-quinolinesulfonyl)piperazine; 4-cyano-3-methylisoquinoline; acetamido-4-cyano-3-methylisoquinoline; 8-bromo-2-monoacyladenosine-3,5-cyclic monophosphorothioate; adenosine 3,5-cyclic monophosphorothioate; 2-O-monobutyl-cyclic adenosine monophosphate; 8-chloro-cyclic adenosine monophosphate; N-[2-(cinnamoylamino acid)]-5-isoquinolinone; reverse phase-8-hexylamino adenosine 3,5-monophosphorothioate; reverse phase-8-piperidinyladenosine-cyclic adenosine monophosphate; reverse phase-adenosine 3,5-cyclic monophosphorothioate; 5-iodotuberculin; 8-hydroxyadenosine-3,5-monophosphorothioate; calphostin C; daphnetin; reverse phase-8-chlorophenyl-cyclic adenosine monophosphate; reverse phase-cyclic adenosine monophosphate; reverse phase-8-Br-cyclic adenosine monophosphate; 1-(5-isoquinolinesulfonyl)-2-methylpiperidine; 8-hydroxyadenosine-3′,5′-monophosphate; 8-hexylaminoadenosine-3′,5′-monophosphate; and reverse phase-adenosine 3′,5′-cyclic monophosphate; and a phospholipase C inhibitor that can be selected from the group consisting of sodium aristolochate; D609 (sodium tricyclodecan-9-yl xanthogenate); D-erythro-dihydrosphingosine; U-73122 (1-(6-((17β-3-methoxyestra-1,3,5(10)-trien-17-yl)amino)hexyl)-1H-pyrrole-2,5-dione); pyrrolidinethiocarbamate; neomycin sulfate; thielavin B; edelfosine; heterocyclyl-substituted anilino phospholipase C inhibitors; DCIC (3,4-dichloroisocoumarin); and calporoside or derivatives of calporoside.

In still another alternative, the additional therapeutic agent is an inhibitor of a GRK. Inhibitors of GRK can be considered as indirect inhibitors of arrestin-2. Inhibitors of GRK include, but are not limited to, nitric oxide donors that donate nitric oxide or a related redox species, including: S-nitrosoglutathione; a C-nitroso compound in which the nitroso moiety is attached to a tertiary carbon; a compound of Formula (N-I):

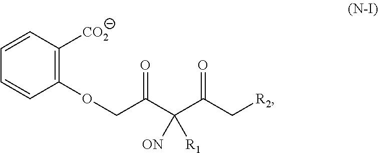

wherein the counterion is hydrogen and wherein R₁ and R₂ are selected from the group consisting of C₁-C₆ alkyl and C₆-C₂₀ aryl, which is optionally substituted with a substituent selected from the group consisting of amino, hydroxyl, or carboxyl, particularly dimeric 2-[4′-(α-nitroso)isobutyrylphenyl]propionic acid; a C-nitroso compound containing a moiety of Formula (N-II):

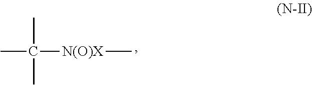

wherein X is S, O, or NR, wherein R is selected from the group consisting of C₁-C₆ alkyl which is unsubstituted or is substituted with one or more alcohol, ether, ester, or amide groups which contain from 1 to 10 carbon atoms; a compound selected from the group consisting of C-nitroso derivatives of acetylsalicylic acid, C-nitroso derivatives of propranolol, C-nitroso derivatives of nadolol, C-nitroso derivatives of carvedilol, C-nitroso derivatives of prazosin, C-nitroso derivatives of tinolol, C-nitroso derivatives of metoprolol, C-nitroso derivatives of pindolol, C-nitroso derivatives of labetalol, C-nitroso derivatives of triamterene, C-nitroso derivatives of furosemide, C-nitroso derivatives of enalapril, C-nitroso derivatives of ramipril, C-nitroso derivatives of lovastatin, C-nitroso derivatives of pravastatin, C-nitroso derivatives of gemfibrozil, C-nitroso derivatives of clofibrate, C-nitroso derivatives of nifedipine, C-nitroso derivatives of amlodipine, C-nitroso derivatives of diltiazem, C-nitroso derivatives of verapamil, C-nitroso derivatives of cimetidine, C-nitroso derivatives of ranitidine, C-nitroso derivatives of albuterol, C-nitroso derivatives of ipratropium bromide, C-nitroso derivatives of memantine, C-nitroso derivatives of 10-deacetylbaccatin III, C-nitroso derivatives of taxol, C-nitroso derivatives of pretomanid, C-nitroso derivatives of dalcetrapib, C-nitroso derivatives of superoxide dismutase mimetics, C-nitroso derivatives of allopurinol, C-nitroso derivatives of celecoxib, C-nitroso derivatives of indomethacin, C-nitroso derivatives of thioflosulide, and C-nitroso derivatives of etodolac; and a compound selected from the group consisting of S-nitrosoglutathione (GSNO), S-nitroso-N-acetylpenicillamine, S-nitroso-cysteine, S-nitroso-cysteine ethyl ester, S-nitroso-cysteinyl glycine, S-nitroso-γ-methyl-L-homocysteine, S-nitroso-L-homocysteine, S-nitroso-γ-thio-L-leucine, S-nitroso-6-thio-L-leucine, S-nitrosoalbumin, sodium nitroprusside (nipride), ethyl nitrite, nitroglycerin, SIN1(molsidomine), furoxamines, and N-hydroxy-(N-nitrosamine).

Yet another aspect of the present invention is a pharmaceutical composition comprising:
(1) a therapeutically effective quantity of nadolol or a derivative or analog of nadolol to inhibit the β-arrestin pathway to treat the pulmonary airway disease; and
(2) a pharmaceutically acceptable carrier.

The pharmaceutical composition can be formulated to treat a pulmonary disease selected from the group consisting of chronic obstructive pulmonary disease (COPD), asthma, bronchiectasis, bronchitis, Churg-Strauss syndrome, pulmonary sequelae of cystic fibrosis, emphysema, allergic rhinitis, pneumonia, and pulmonary symptoms associated with infection with SARS-CoV-2. In particular, the pharmaceutical composition can be formulated to treat COPD.

Preferably, the composition comprises nadolol. However, in an alternative, the composition can comprise a derivative or analog of nadolol or a prodrug of nadolol as described above.

Typically, administration of the pharmaceutical composition exerts a therapeutic effect that is a reduction in pulmonary airway constriction hyperresponsiveness. Also typically, administration of the pharmaceutical composition exerts a therapeutic effect that is an upregulation of pulmonary 2-adrenergic receptors. Also typically, administration of the pharmaceutical composition exerts a therapeutic effect that is increased pulmonary airway relaxation responsiveness to 2-adrenergic agonist drugs.

Typically, the pharmaceutical composition is formulated for administration by a route selected from the group consisting of oral, sustained-release oral, parenteral, sublingual, buccal, administration by insufflation, and administration by inhalation. Preferably, the pharmaceutical composition is formulated for administration by the sustained-release oral route, particularly when the pharmaceutical composition comprises nadolol.

In one alternative, administration of the pharmaceutical composition results in continuous levels of the nadolol or the derivative or analog of nadolol in the bloodstream, particularly when the composition is formulated for administration by the sustained-release oral route.

When the pharmaceutical composition comprises nadolol, in one alternative, the quantity of nadolol in the composition is selected from the group consisting of 1 mg, 3 mg, 5 mg, 10 mg, 15 mg, 30 mg, 50 mg, and 70 mg per unit dose.

The composition can further comprise at least one additional therapeutic agent for the treatment of the pulmonary airway disease, as described above with respect to methods for treatment of the pulmonary airway disease. Typically, the at least one additional therapeutic agent is for treatment of chronic obstructive pulmonary disease.

The composition can further comprise a therapeutically effective quantity of a β₂-selective adrenergic agonist as described above.

Alternatively, the composition can further comprise a therapeutically effective quantity of a corticosteroid as described above.

Alternatively, the composition can further comprise a therapeutically effective quantity of a biological as described above.

In another alternative, the composition can further comprise a therapeutically effective quantity of an anticholinergic drug as described above.

In yet another alternative, the composition can further comprise a therapeutically effective quantity of a xanthine compound as described above.

In still another alternative, the composition can further comprise a therapeutically effective quantity of an anti-IgE antibody as described above.

In still another alternative, the composition can further comprise a therapeutically effective quantity of a leukotriene antagonist as described above.

In still another alternative, the composition can further comprise a therapeutically effective quantity of a phosphodiesterase IV inhibitor.

In still another alternative, the composition can further comprise a therapeutically effective quantity of a 5-lipoxygenase inhibitor as described above.

In still another alternative, the composition can further comprise a therapeutically effective quantity of a mast cell stabilizer as described above.

In still another alternative, the composition can further comprise a therapeutically effective quantity of an arrestin-2 inhibitor, such as, but not limited to: a protein fragment of arrestin-2; a compound of Formula (A-I) as described above; an omega-3 fatty acid as described above; a CXCR2 inhibitor as described above; a MyD88 inhibitor as described above; a MD2 inhibitor as described above; inositol hexaphosphate; or barbadin.

In still another alternative, the composition can further comprise a therapeutically effective quantity of an inhibitor of a GRK as described above. Inhibitors of a GRK can act as indirect inhibitors of arrestin-2.

In still another alternative, the composition can further comprise a therapeutically effective quantity of an inhibitor of protein kinase A as described above. Inhibitors of protein kinase A can act as indirect inhibitors of arrestin-2.

In still another alternative, the composition can further comprise a therapeutically effective quantity of an inhibitor of phospholipase C as described above. Inhibitors of phospholipase C can act as indirect inhibitors of arrestin-2.

In compositions according to the present invention, typically, the pharmaceutically acceptable carrier is selected from the group consisting of a solvent, a dispersion medium, a coating, an antibacterial agent, an antifungal agent, an isotonic agent, an absorption delaying agent, a preservative, a sweetening agent for oral administration, a thickening agent, a buffer, a liquid carrier, a wetting, solubilizing, or emulsifying agent; an acidifying agent, an antioxidant, an alkalinizing agent, a carrying agent, a chelating agent, a colorant, a complexing agent, a suspending or viscosity-increasing agent, a flavor or perfume, an oil, a penetration enhancer, a polymer, a stiffening agent, a protein, a carbohydrate, a bulking agent, and a lubricating agent.

The invention is illustrated by the following Examples. These Examples are included for illustrative purposes only, and are not intended to limit the invention.

### EXAMPLES

### Example 1

### Airway Resistance Reduction by Chronic Administration of 13-Adrenergic Inverse Agonists

### Methods

Balb/cJ mice aged 6 weeks (Jackson Animal Laboratory, Bar Harbor, Maine) were housed under specific pathogen-free conditions and fed a chicken ovalbumin-free diet. The effects of administration of the non-selective β-adrenergic inverse agonists carvedilol (GlaxoSmithKline, King of Prussia, PA) and nadolol (Sigma Chemical, St. Louis, MO) and of salbutamol (Sigma Chemical, St. Louis, MO), a β₂-adrenergic partial agonist, were examined in a murine model that exhibited cardinal features of human asthma, such as pulmonary eosinophilic inflammation, airway hyperresponsiveness, and heterogeneous airway narrowing. The results obtained in drug-treated animals were compared with those obtained in vehicle-treated counterparts (controls) in experiments performed in close temporal relationship. The outcome measures of the study of Example 1 included statistically-significant differences between drug-treated mice and non-treated animals in terms of baseline airway resistance, degree of airway responsiveness to cholinergic stimulation, and bronchoalveolar lavage (BALF) cellularity. Mice were sensitized with subcutaneous injection of 25 μg of ovalbumin adsorbed to aluminum hydroxide on protocol days 2, 9, and 16. Subsequently, mice were given 50 μL of saline solution containing 25 μg of ovalbumin intranasally, on a daily basis, from protocol days 23 through 27. A group of ovalbumin-sensitized saline-challenged mice serves as controls for systemic sensitization and respiratory challenges with ovalbumin. Prior to intranasal administrations, mice were sedated with halothane vapor. For the study of Example 1, ovalbumin-sensitized and ovalbumin-challenged mice, and ovalbumin-sensitized and saline-challenged mice will be referred to as asthmatic mice and control mice, respectively. The drugs used were salbutamol (a β₁/β₂-adrenergic agonist), alprenolol (a β₁/β₂-adrenergic antagonist with partial agonist activity), and nadolol and carvedilol (both non-selective β₁/(β₂ adrenergic inverse agonists).

To examine the effects of duration of β-adrenergic ligand therapy on the phenotype of the murine model of asthma, the experimental drugs were administered either acutely or chronically to different groups of asthmatic mice.

Asthmatic mice on acute therapy were given a single intravenous bolus infusion of either β-adrenergic drug or normal saline on protocol day 28, 15 minutes before airway responsiveness to methacholine was determined. The doses of carvedilol, nadolol, alprenolol, and salbutamol administered to the mice were 24 mg/kg, 72 mg/kg, 72 mg/kg, and 0.15 mg/kg, respectively. Asthmatic mice on chronic therapy were treated with the β-adrenergic drug during protocol days 1 to 28. Those on β-antagonists had free access to chow treated with carvedilol, nadolol, or alprenolol at concentrations of 2400 ppm, 250 ppm, or 7200 ppm, respectively. These concentrations were chosen based on those producing therapeutic effects in mice in previously published studies. The non-asthmatic mice were fed normal chow. Salbutamol was delivered for 28 days at a dose of 0.5 mg/kg/day using an osmotic minipump (Alzet®, #2004, Durect Corporation, Cupertino, CA).

On protocol day 28, mice were anesthetized, tracheotomized, and connected to a computer-controlled small animal ventilator (Flexivent, Scientific Respiratory Equipment, Inc., Montreal, Canada). Airway resistance (Raw) was measured using the forced oscillation technique. The cellular composition of bronchoalveolar lavage fluid (BALF) was also determined. In non-treated asthmatic mice, the degree of airway responsiveness and the number of eosinophils recovered in BALF were significantly higher compared to the ovalbumin-sensitized saline-challenged (control) mice. However, it was observed that the degree of airway responsiveness and the number of eosinophils recovered in BALF were lower in non-treated asthmatic mice studied in close temporal relationship with mice receiving acute β-adrenergic antagonist treatments that in those obtained in non-treated asthmatic mice studied concomitantly with mice on chronic β-adrenergic antagonist therapy.

To induce airway constriction, a solution containing 150 μg/mL of acetyl-α-methylcholine chloride (methacholine) (Sigma Chemical, St. Louis, MO) was infused intravenously at constant rates using a syringe infusion pump (Raze Scientific Instruments, Stanford, CN). The methacholine infusion was started at 0.008 mL/min, and its rate was doubled stepwise up to a maximum of 0.136 mL/min. Each methacholine dose was administered for 3 to 5 minutes, during which data were sampled at 1-minute intervals and then averaged.

### Data Analysis

The complex input impedance of the respiratory system was computed and the value of the real part of respiratory system impedance at 19.75 Hz was taken to reflect the magnitude of airway resistance (R_{aw}). To examine the degree of airway responsiveness of each animal, the values for R_{aw} as a function of methacholine doses were plotted. The largest value for R_{aw} achieved in response to methacholine stimulation was referred to as R_{awpeak}. For mice that achieved a plateau in the methacholine dose-R_{aw} response curve, the ED₅₀ was calculated by linear interpolation using the GraphPad Prism4 (GraphPad Software, Inc.). Results obtained for β-adrenergic drug-treated and non-treated mice were performed using the analysis of variance for multiple groups of a Student's t-test for comparing two groups. The Bonferroni test was used to examine the statistical differences between experimental groups. The effects of acute drug treatments on baseline respiratory system mechanics were assessed using a two-tailed paired t-test. A value of P<0.05 was considered significant.

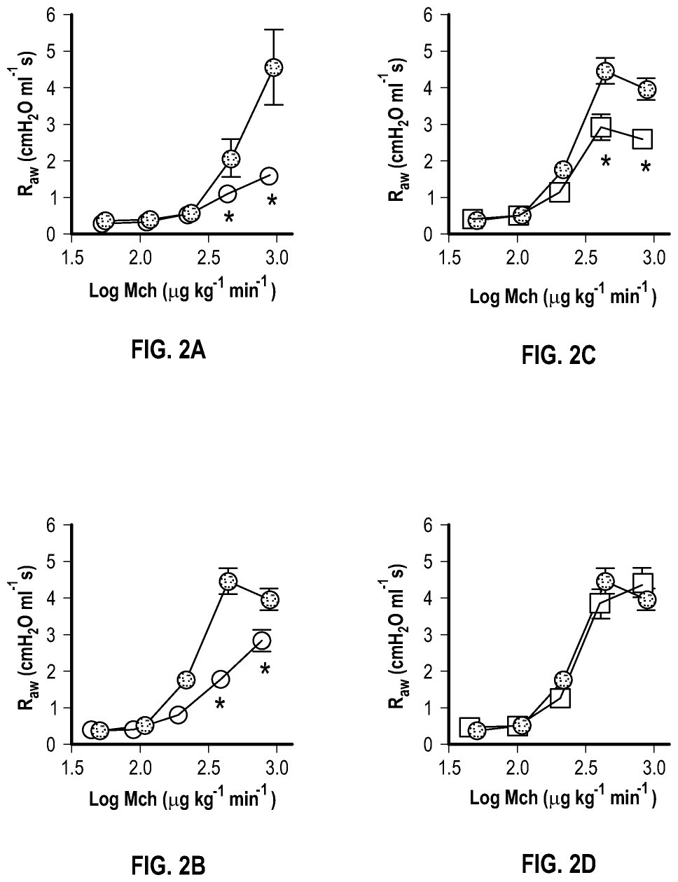

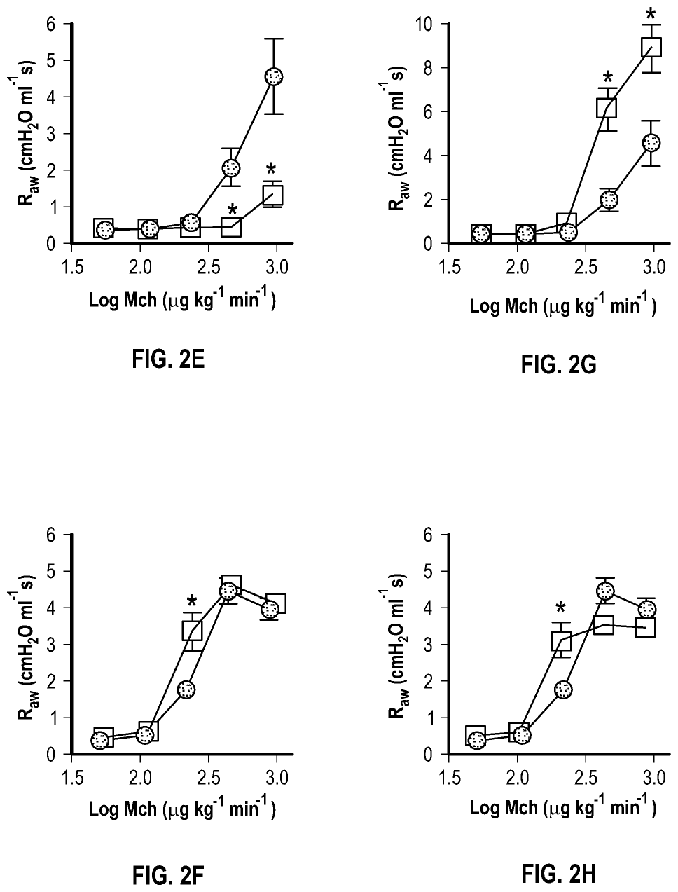

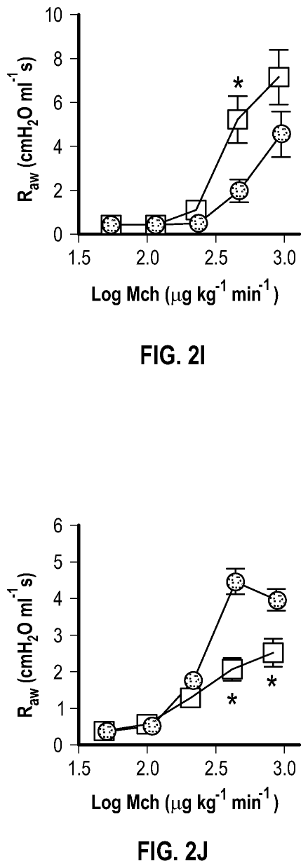

### FIG. 2

FIGS. 2A and 2B show that methacholine provocation significantly enhances airway resistance (R_{aw}) in asthmatic mice in contrast to a minimal response upon saline provocation of asthmatic mice. This demonstrates that the mouse model in this study exhibits airway hyperresponsiveness, a key feature of airway dysfunction in human asthma, as well as in human chronic obstructive pulmonary disease and other pulmonary diseases or conditions associated with hyperinflammation.

In FIG. 2C, the administration of a single intravenous bolus of salbutamol to asthmatic mice reduced the level of airway responsiveness to methacholine provocation and the level of airway resistance as expected, thus demonstrating an acute effect of this agent. However, in FIG. 2D, when salbutamol was delivered for 28 days to the mice, no protection was observed. This lack of reduction of airway hyperresponsiveness upon chronic administration of a β-adrenergic agonist has been observed in humans when tolerance to these drugs develop. The issue of tolerance to such β-adrenergic agonists is also relevant with respect to the administration of these drugs for the treatment of chronic obstructive pulmonary disease.

In FIG. 2E, when asthmatic mice were given a single intravenous bolus of alprenolol, a β-adrenergic antagonist with partial agonist activity, their airway responsiveness was diminished, as indicated by significant decreases in both the values for R_{aw} at methacholine doses ≥408 μg/kg/min (P<0.05) compared with those obtained in non-treated counterparts. The reduction in airway responsiveness upon acute administration of alprenolol is similar to that observed for salbutamol, consistent with the partial agonist activity that alprenolol possesses. In FIG. 2F, when asthmatic mice were exposed to alprenolol for 28 days, their average methacholine dose-response relationship was similar to that obtained in nontreated mice, demonstrating that this drug provides no benefit upon chronic administration, as is the case with salbutamol. This is again directly analogous to the tolerance seen in human patients after long-term administration of such drugs, including when such drugs are administered for the treatment of chronic obstructive pulmonary disease.

In FIG. 2G, a single intravenous bolus of carvedilol enhanced the airway responsiveness in the asthmatic mice. This is consistent with previous observations in humans that acute delivery of β-adrenergic antagonists to asthmatics can result in severe airway constriction. In contrast, in FIG. 2H, chronic administration of carvedilol reduced the responsiveness of asthmatic mice to methacholine provocation.

In FIG. 2I, a single intravenous bolus of nadolol also enhanced the airway responsiveness of asthmatic mice similar to the result observed for carvedilol. Chronic delivery of nadolol, as shown in FIG. 2J, also produced a decrease in airway responsiveness, which was more pronounced than that caused by carvedilol treatment. Indeed, the average methacholine dose-R_{aw} response relationship obtained in asthmatic mice on chronic nadolol treatment was similar to that obtained in mice on acute salbutamol treatment.

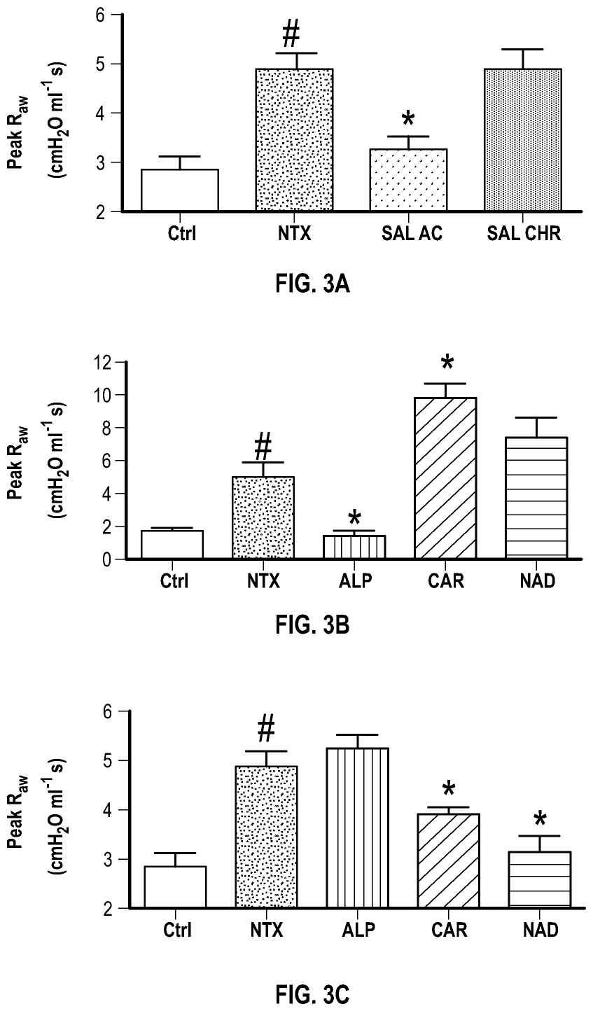

### FIG. 3

FIG. 3 shows the effects of administration of β-adrenergic receptor ligands on the peak airway responsiveness to cholinergic stimulation in asthmatic mice. Peak R_{aw} was determined for each mouse by examining the individual methacholine dose-response curves and choosing the highest R_{aw} value produced by any of the methacholine doses (most often the next to last dose, 408 μg kg⁻¹ min⁻¹). Shown are the mean peak R_{aw}±SEM after treatments with the β-adrenergic receptor agonist salbutamol (A), after acute treatments with various agents (B) (ALP=alprenolol; CAR=carvedilol; NAD; nadolol); and after chronic treatments with the same agents used in (B), all in comparison to nontreated asthmatic mice (NTX) (black bars, n=7-25) and control mice (Ctrl, white bars, n=6-21). Values are mean±SEM for the peak R_{aw} values to methacholine of n=8-19 mice. Note the change in scale of the y-axis for (B). *, P<0.05 compared to NTX; #, P<0.05 compared to Ctrl (ANOVA).

The results of Example 1 are applicable to chronic obstructive pulmonary disease as well as to asthma as both diseases involve exacerbations of airway resistance and are associated with the consequences of hyperinflammation.

### Example 2

### Chronic Inverse Agonist Treatment Increases 13-Adrenergic Receptor Numbers as Measured by Radioligand Binding

β₂-adrenergic receptor numbers were measured in asthmatic mice as follows. Asthmatic mice (ovalbumin-challenged) were treated as follows: Ctrl, no drug treatment with methacholine challenge; salbutamol, a short-acting β₂ agonist; carvedilol, a β₁, β₂, non-selective inverse agonist with α₁-adrenergic antagonist activity; nadolol, a highly specific, hydrophilic β₁, β₂, non-selective inverse agonist; and alprenolol, a β-adrenergic antagonist. Drug treatments were either a single treatment 15 minutes prior to methacholine challenge or ongoing for 28 days (salbutamol was delivered continuously via a subcutaneous osmotic minipump and alprenolol, carvedilol, and nadolol were in animal chow). Mice were sacrificed and lung membranes were isolated as follows. Frozen lung tissue was homogenized in an ice-cold buffer containing 0.32 M sucrose and 25 mM Tris (pH 7.4) using a polytron (Pro 200, Pro Scientific, Inc.). The homogenate was centrifuged at 1000×g for 10 min at 4° C. The resulting supernatant was centrifuged at 40,000×g for 20 min at 4° C. The pellet was suspended in an ice-cold 25 mM Tris-HCl buffer (pH 7.4) and centrifuged at 40,000×g for 20 min at 4° C. The final pellet was suspended in 200 μL of 25 mM Tris-HCl (pH 7.4); membrane protein concentration was determined by BCA protein assay kit. Radioligand receptor binding incubation mixtures contained membranes (˜10 μg of protein), (−)3-[¹²⁵I] cyanopindolol (ICYP) in 25 mM Tris-HCl, pH 7.4, in increasing concentrations (5-7500 pM) and binding buffer in a final volume of 250 μL. Propranolol was used to determine nonspecific binding. The incubation was done at 37° C. for 2 h and terminated by rapid vacuum filtration through glass fiber filters. The filters were washed three times with 250 μL of cold wash buffer (25 mM Tris-HCl, pH 7.4) and the radioactivity determined in a counter. All experiments were performed in triplicate and values are mean±SEM of n=3-5 animals in each group. Receptor densities are expressed as femtomoles of sites per milligram of protein. Bₘₐₓ is determined by nonlinear regression of the saturation binding curves. Apparent KD values (in parentheses) are expressed as pM. Please note that the 15-minute and 28-day time points refer to duration of drug treatment. All mice were killed at the same age and thus for vehicle treated groups (Ctrl and NTX) the groups were identical and the results pooled. #P<0.05 compared to Ctrl; *P<0.05 compared to NTX (Student's t-test).

Radioligand binding revealed that 2-adrenergic receptor levels appear to be somewhat lower in methacholine-challenged but otherwise untreated asthmatic mice as compared with untreated, unchallenged mice, as shown in Table 1. Chronic alprenolol treatment led to a slight decrease of the level of the 2-adrenergic receptor. The same was true of chronic salbutamol treatment. Most significantly, the carvedilol-treated mice demonstrated an over 10-fold increase of the level of β₂-adrenergic receptors over the non-treated mice, demonstrating the efficacy of this β-adrenergic inverse agonist in increasing receptor levels upon chronic administration. Similarly, the nadolol-treated mice demonstrated a nearly eightfold increase of the level of receptors over the untreated methacholine-challenged asthmatic mice.

TABLE 1
Determination of β-Adrenergic Receptor Density by Radioligand Binding

| | 15 Minutes | | 28 Days | |
|---|---|---|---|---|
| Treatment | Bₘₐₓ | K_{D} | βₘₐₓ | K_{D} |
| Ctrl | 286.8 ± 88.02 | (107.9 ± 43.67) | 286.8 ± 88.02 | (107.9 ± 43.67) |
| NTX | 109.2 ± 9.72 # | (193.6 ± 20.66) | 109.2 ± 9.72 # | (193.6 ± 20.66) |
| Salbutamol | 256.5 ± 29.24* | (228.8 ± 33.07) | 97.0 ± 23.02 | (225.4 ± 41.79) |
| Alprenolol | 299.5 ± 12.19* | (453.6 ± 86.33) | 179.2 ± 53.05 | (290.9 ± 55.07) |
| Carvedilol | 86.3 ± 19.42 | (565.2 ± 192.8)* | 904.1 ± 43.46* | (1444.0 ± 202.0) |
| Nadolol | 181.9 ± 48.28 | (695.1 ± 286.3)* | 785.5 ± 154.8* | (1591.6 ± 335.0)* |

The results of Example 2 are relevant to chronic obstructive pulmonary disease as the density of β₂-adrenergic receptors is important for response to agonists used for the treatment of not only chronic obstructive pulmonary disease but other respiratory diseases and conditions as described above.

### Example 3

### Chronic Inverse Agonist Treatment Increases β-Adrenergic Receptor Numbers as Monitored by Immunochemistry

For immunohistochemistry analysis of β₂-adrenergic receptor levels, non-drug-treated control mice and mice treated chronically with the 2-adrenergic inverse agonist nadolol were used. The mice were sacrificed and the lungs excised. Then the lungs were fixed in 4% paraformaldehyde (45 min, 0° C.). After fixation, lungs were washed in PBS (60 min) and placed in increasing concentrations of sucrose (10% sucrose/5% glycine in PBS for 30 min; 20% sucrose/10% glycine in PBS for 30 min; 30% sucrose/15% glycine in PBS for 12 h at 4° C.). Lungs were embedded in OCT and 12-μm sections cut with a Tissue-Tek II cryostat. The sections were air-dried and fixed with 4% paraformaldehyde for 15 minutes. After 3 washes in PBS, the slides were blocked with 5% milk in PBS for 1 hour, and then incubated overnight with anti-β₂-adrenergic receptor antibody (1:200, Santa Cruz Biotechnology) in blocking solution. Slides were washed in PBS and incubated with secondary antibody (1:200, Cy3 goat anti-rabbit antibody, 16 h at 4° C.). Control slides were incubated with antibody specific blocking peptide to demonstrate specificity of binding of the primary antibody. After washing with PBS, coverslips were mounted and viewed by epifluorescent microscopy.

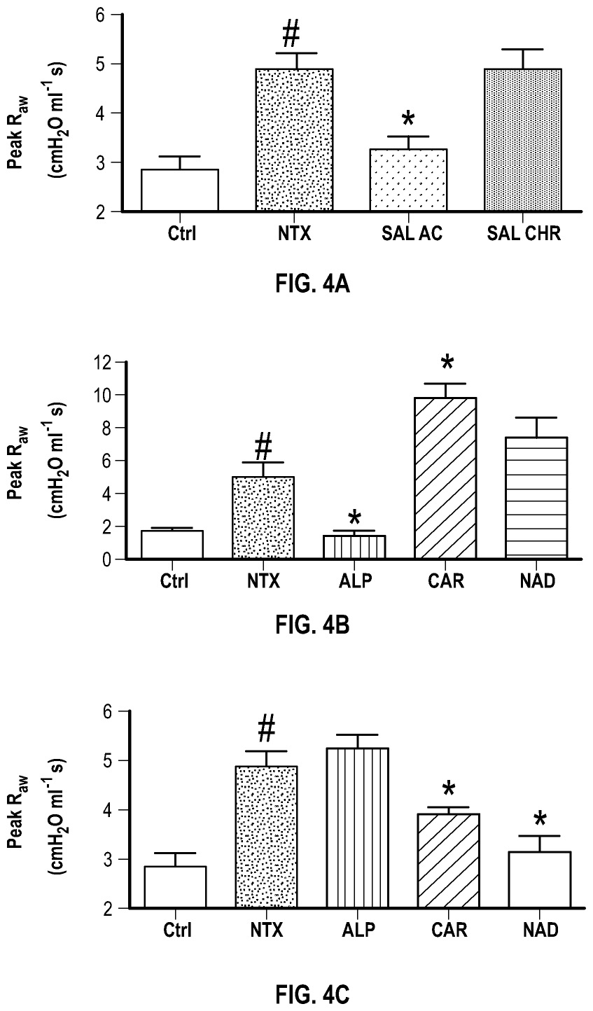

As shown in FIG. 4, labeling with anti-β₂-adrenergic receptor antibodies was considerably more intense in lungs from animals treated with nadolol than in lungs from untreated animals (A, control+antibody; B, control+antibody+blocking peptide; C, nadolol+antibody; D, nadolol+antibody+blocking peptide). Loss of this signaling upon incubation in the presence of the 2-adrenergic receptor peptide demonstrates that this antibody is specifically binding the 2-adrenergic receptor. This observation is consistent with the radioligand binding data of Example 2 and suggests that β₂-adrenergic receptors are effectively upregulated by chronic administration of β₂-adrenergic inverse agonist drugs.

### Example 4

### Effect of Combination of Carvedilol and Salbutamol on Airway Hyperresponsiveness

The effect of combination therapy with carvedilol and salbutamol was compared to monotherapy with carvedilol alone on airway hyperresponsiveness in asthmatic mice.

Mice (Balb/cJ) aged 6 weeks were housed under specific pathogen-free conditions and fed a chicken ovalbumin-free diet. Mice were systemically sensitized with ovalbumin adsorbed to aluminum hydroxide. Mice were treated as follows: CAR/SAL 28D=for 28 days mice (n=6-12) were administered carvedilol (2400 ppm in animal chow) and salbutamol (subcutaneous delivery of 0.5 mg/kg/day in an Alzet #2400 osmotic minipump); NTX S/C=mice (n=6-12) no drug treatment for 28 days; CTRL=mice (n=6-12) no drug treatment for 28 days, not subsequently challenged; CARHD 28D=for 28 days mice (n=6-12) were administered carvedilol only (2400 ppm in animal chow); CARHD 28D SAL AC=for 28 days mice (n=6-12) were administered carvedilol (2400 ppm in animal chow) and 15 minutes prior to measuring airway hyperresponsiveness, salbutamol was administered at a dose of 1.2 mg/kg.

To measure airway hyperresponsiveness after 28 days, all mice except the CTRL (control) mice were challenged with ovalbumin and then all mice were anesthetized, tracheotomized, and connected to a Flexivent small animal ventilator to measure airway resistance (R_{aw}) by the forced oscillation technique. To induce airway constriction, a solution containing 150 μg/mL of methacholine was infused using a syringe infusion pump. The methacholine infusion was started at 0.008 mL/min and its rate was doubled stepwise up to a maximum 0.136 mL/min. Each methacholine dose was administered until a plateau was reached, during which data were sampled at 1-minute intervals for 3-5 minutes and then averaged.

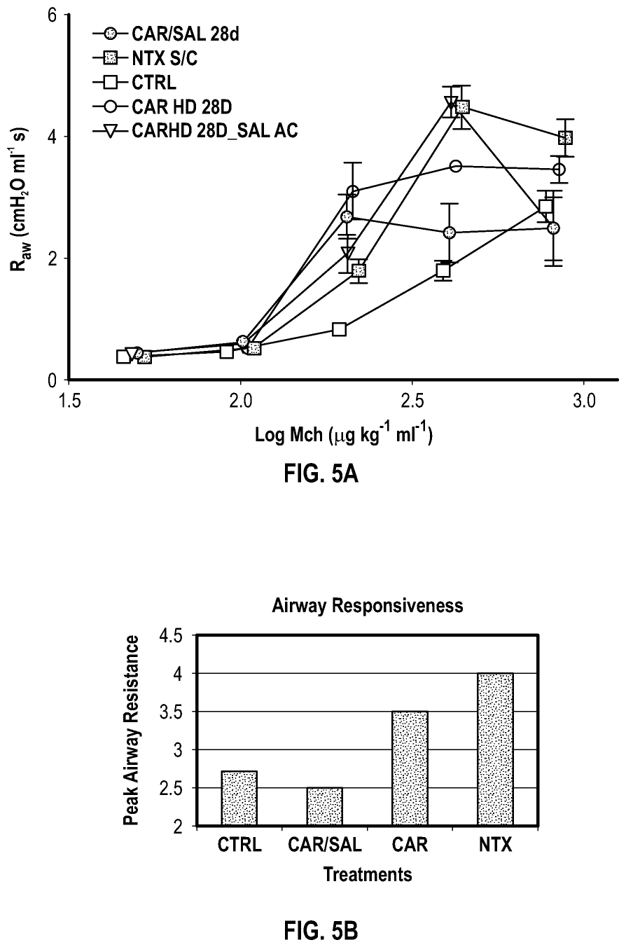

In FIG. 5A, at the highest dose of methacholine, both of the combination drug treatments were equally effective in preventing bronchoconstriction and not statistically significantly different from the control mice which were only challenged with saline solution. The carvedilol monotherapy resulted in higher bronchoconstriction than these treatments but less than the non-drug treated sensitized and challenged (NTX S/C) mice. Thus, the combination therapy of β₂-adrenergic inverse agonist and agonist with the agonist administered either chronically or acutely is effective at ameliorating airway hyperresponsiveness to allergen and methacholine challenge and is an improvement over the monotherapy of the 2-adrenergic inverse agonist alone.

This data is summarized in FIG. 5B, which shows that the combination of carvedilol and salbutamol is the most effective in reducing airway hyperresponsiveness of the treatments for which the results are shown in FIG. 5A. This indicates the effectiveness of the use of combination therapy of β₂-adrenergic inverse agonist and agonist.

### Example 5

### Effect of Combination Therapy with Aminophylline on Acute Airway Effects of Nadolol

Mice were sensitized to the allergen ovalbumin as described in Example 1. Mice were then challenged with allergen and then subjected to methacholine-induced bronchoconstriction challenge, non-drug treated, NTX S/C, or pretreated with nadolol at 0.72 mg/kg i.p. for 15 minutes prior to methacholine challenge (nadolol acute treatment).

At time point 1 (time=−10 min) baseline airway resistance of the mice was determined. At time point 2 (time=−5 min) methacholine was infused into mice to reach their EC70. At time point 3 (time=0 min) aminophylline was administered i.p. at a dose of 100 mg/kg.

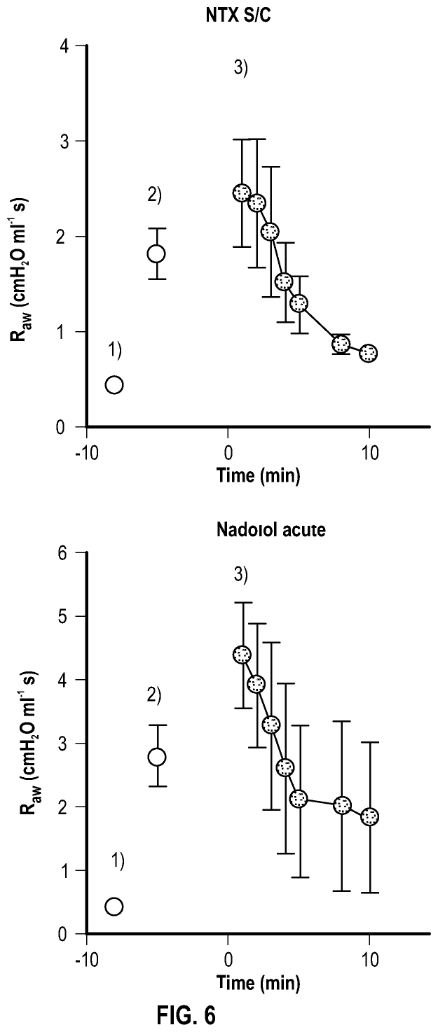

In FIG. 6, pretreatment of mice with nadolol resulted in the same baseline airway resistance as non-drug treated sensitized and allergen-challenged mice. However, upon methacholine challenge, the nadolol-treated mice exhibited a much higher airway resistance of -4.5 versus 2.5 units. Upon administration of aminophylline, there was a significant and sustained drop in airway resistance in both the untreated and the nadolol-treated mice.

Z. Callaerts-Vegh et al., “Effects of Acute and Chronic Administration of β-Adrenoceptor Ligands on Airway Function in a Murine Model of Asthma,” Proc. Natl. Acad. Sci. USA 101: 4948-4953 (2004), have shown that while nadolol administered chronically prevents airway hyperresponsiveness in the same mouse asthma model, nadolol administered acutely worsens airway hyperresponsiveness. These data demonstrate that the addition of the methylxanthine aminophylline can alleviate the acute effects on airway hyperresponsiveness of nadolol administration. This is beneficial in that the opportunity exists for asthma subjects to take nadolol chronically to prevent bronchoconstriction. These subjects then can co-administer a methylxanthine such as aminophylline to prevent the acute detrimental effects of nadolol. These effects are temporary in duration, but can impair patient compliance with a therapeutic regimen.

### Example 6

### Effect of Treatment with Salbutamol or Nadolol on the Ratio of Phospholipase C to Actin in Cultured Tracheal Smooth Muscle Cells

Cultured tracheal smooth muscle cells were obtained from mice exposed to the following treatments: NS/NC=nonasthmatic, non-challenged mice; S/C=asthmatic mice; Sal.Ac=asthmatic mice, acute salbutamol treatment; Sal.Ch=asthmatic mice, chronic salbutamol treatment; Nad.Ac=asthmatic mice, acute nadolol high dose treatment; and Nad.Ch=asthmatic mice, chronic nadolol high dose treatment.

After airway function experiments, the trachea were surgically removed from anesthetized mice that had been treated with drugs or vehicle. The trachea was minced and the cells plated and grown in culture. The smooth muscle cells grow faster and take over the culture dish. The cells were grown in medium which contained the drugs used in the treatment or vehicle controls. Phospholipase C (PLC-β1) was determined by immunoblotting with an antibody specific for the enzyme. Actin was used as a loading control and the amount of PLC-β1 was expressed as a ratio to actin.

The enzyme phospholipase C plays a key role in the pathway leading to asthmatic symptoms, as it cleaves a phosphodiester bond in membrane phospholipids, resulting in the formation of a 1,2-diglyceride. Arachidonate is then released from the diglyceride by the sequential actions of diglyceride lipase and monoglyceride lipase. Once released, a portion of the arachidonate is metabolized rapidly, leading to oxygenated products, including eicosanoids such as prostaglandins. Thus, any treatment that can inhibit phospholipase C activity is relevant for the treatment of asthma. Similarly, inhibitors of phospholipase C are also relevant for the treatment of other respiratory diseases and conditions such as chronic obstructive pulmonary diseases.

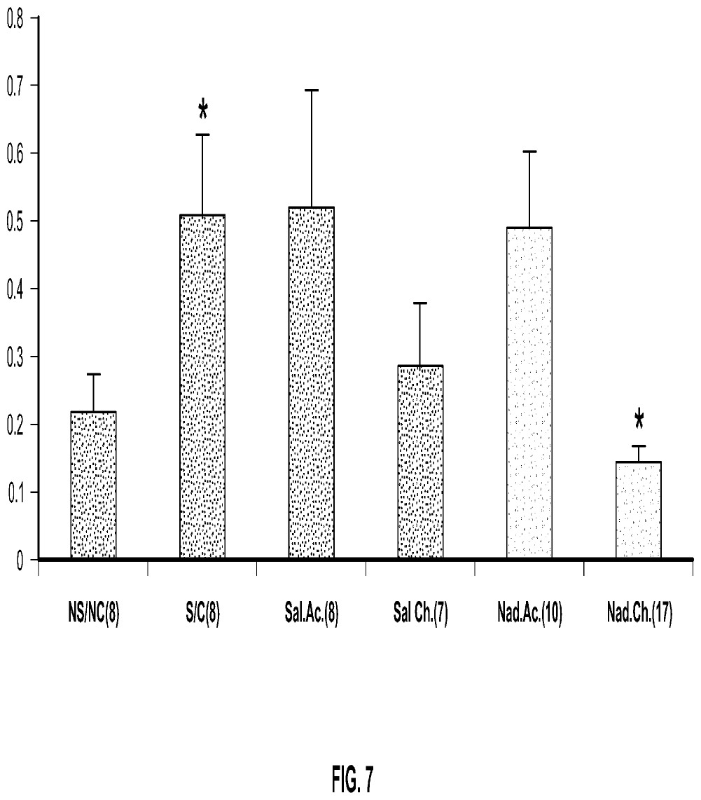

The results are shown in FIG. 7. The results shown in FIG. 7 indicate that chronic administration of nadolol significantly decreases the activity of phospholipase C. This indicates that such chronic administration of nadolol is effective against asthma, chronic obstructive pulmonary disease, and other diseases and conditions affecting the respiratory tract and associated with inflammation and airway hyperresponsiveness and prevents activation of some of the mechanisms that lead to the symptoms of asthma and other such conditions.

### Example 7

### Effect of 13-Adrenergic Receptor Drugs at Low and High Doses on Airway Resistance

For these experiments, salbutamol was used for chronic administration at 0.5 mg/kg/day with a minipump and for acute administration at 0.15 mg/kg by i.v. bolus 15 minutes prior to challenge. Alprenolol was used at a high dose of 7200 ppm in chow or at a low dose of 720 ppm in chow. Carvedilol was used at a high dose of 2400 ppm in chow or at a low dose of 720 ppm in chow. Nadolol was used at a high dose of 250 ppm in chow or at a low dose of 25 ppm in chow. Nadolol was also tested at 1 ppm in chow and these results were identical to the untreated mice.

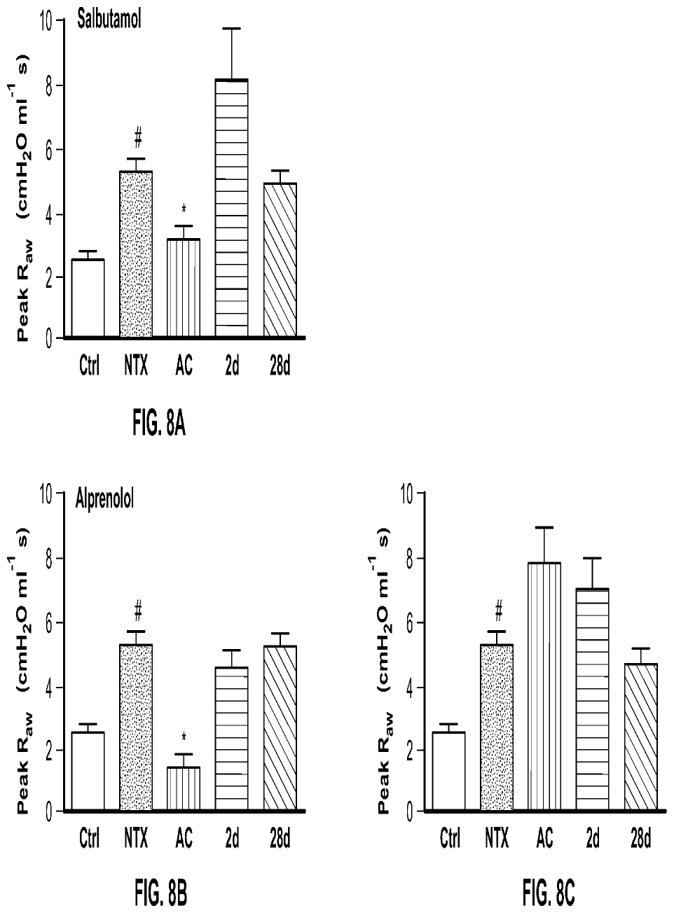

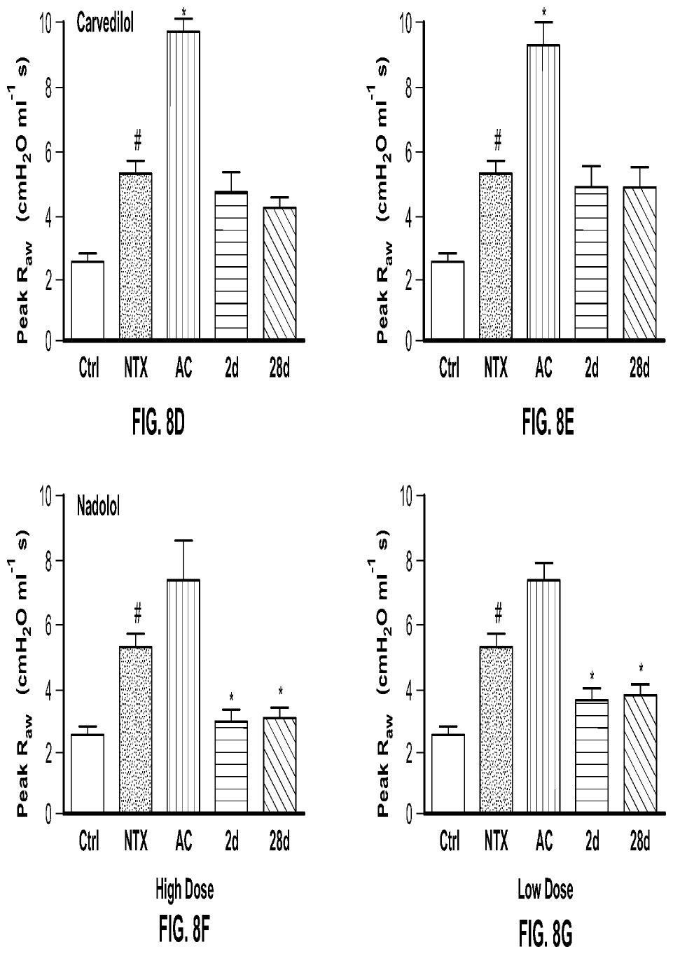

The results are shown in FIGS. 8A (salbutamol); 8B (high-dose alprenolol); 8C (low-dose alprenolol); 8D (high-dose carvedilol); 8E (low-dose carvedilol); 8F (high-dose nadolol); and 8G (low-dose nadolol). In these diagrams, Ctrl=control mice, non-asthmatic, non-drug treated; NTX=asthmatic mice, non-drug treated; AC=acute administration; 2d=chronic administration for 2 days; 28d=chronic administration for 28 days. The airway resistance (R_{aw}) is plotted as cm H₂O ml⁻¹ s. The data particularly shows the effect of the β-adrenergic inverse agonists carvedilol and nadolol in providing protection from airway hyperresponsiveness with chronic administration.

### Example 8

### Correlation of Decrease in Airway Resistance with Upregulation of β-Adrenergic Receptor Density

The correlation of the decrease in airway resistance with the upregulation of β-adrenergic receptor density for three different periods of administration of salbutamol, alprenolol, carvedilol, and nadolol is shown in Table 2. The periods of administration of the agents are 15 minutes, 2 days, and 28 days. Only the inverse agonists carvedilol and nadolol showed an increase in βadrenergic receptor density at periods longer than 15 minutes; carvedilol showed an increase in receptor density at 28 days, while nadolol showed an increase in receptor density at both 2 days and 28 days. There was an exact correlation between the decrease of airway resistance (R_{aw}) and the increase in receptor density. This strongly supports the concept of combination therapy, such as with an inverse agonist and an agonist.

TABLE 2
Correlation of Decrease in Airway Resistance with
Upregulation of β₂-Adrenergic Receptor Density

| | 15 minutes | | 2 days | | 28 days | |
|---|---|---|---|---|---|---|
| | | Increased | | Increased | | Increased |
| | Decreased | β₂AR | Decreased | β₂AR | Decreased | β₂AR |
| | R_{aw} | density | R_{aw} | density | R_{aw} | density |
| Salbutamol | yes | yes | no | no | no | no |
| Alprenolol | yes | yes | no | no | no | no |
| Carvedilol | no | no | no | no | yes | yes |
| Nadolol | no | no | yes | yes | yes | yes |

### Example 9

### Effect of Chronic Treatment with Metoprolol and Timolol on Airway Responsiveness in Asthmatic Mice

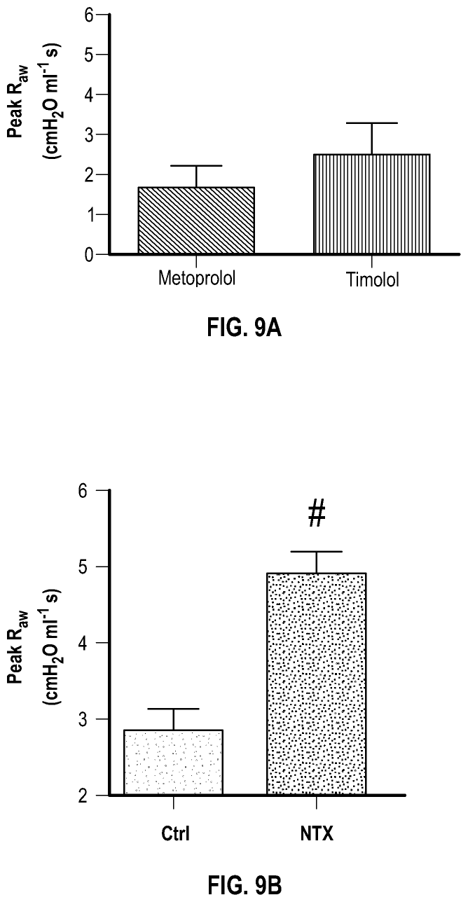

The protocols of Example 1 were followed for two additional inverse agonists, metoprolol (dosage of 20 mg/kg administered 3×daily via subcutaneous injection for 7 days) and timolol (dosage of 20 mg/kg in chow for 7 days), using asthmatic mice and methacholine challenge as in Example 1. Airway resistance (R_{aw}) was measured as in Example 1. The results for metoprolol and timolol are shown in FIG. 9A. The results were compared to historical controls as shown in FIG. 9B: Ctrl, no drug treatment, no challenge with methacholine; NTX, no drug treatment, challenged with methacholine. The results indicate that chronic treatment with both metoprolol and timolol are effective in reducing airway hyperresponsiveness in asthmatic mice.

### Example 10

### Administration of Nadolol Prevents Mucous Metaplasia

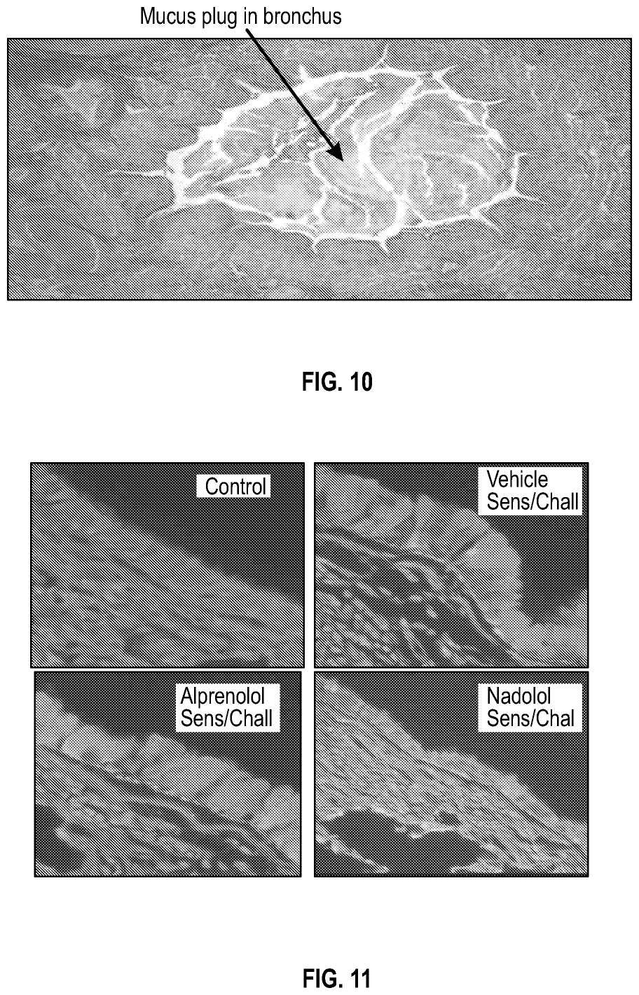

The occurrence of mucous metaplasia can lead to severe consequences in asthma and other airway diseases associated with chronic airway obstruction. FIG. 10 is a photomicrograph showing the occurrence of a mucus plug in the bronchus of an 8-year-old girl with fatal asthma.

FIG. 11 is a series of photomicrographs showing that nadolol is effective in preventing mucous metaplasia while the antagonist alprenolol is ineffective in preventing mucous metaplasia: top left, control; top right, sensitized/challenged mice without treatment showing mucous metaplasia; bottom left, sensitized/challenged mice after treatment with alprenolol showing no improvement in mucous metaplasia; bottom right, sensitized/challenged mice after treatment with nadolol showing nearly complete elimination of mucous metaplasia.

Therefore, nadolol is highly effective in eliminating or preventing mucous metaplasia which, in turn, can prevent serious consequences that may otherwise occur due to the accumulation of mucus in the respiratory tract.

### Example 11

### Nadolol Reverses Epithelial Changes Via Inhibition of the Beta-Arrestin Pathway

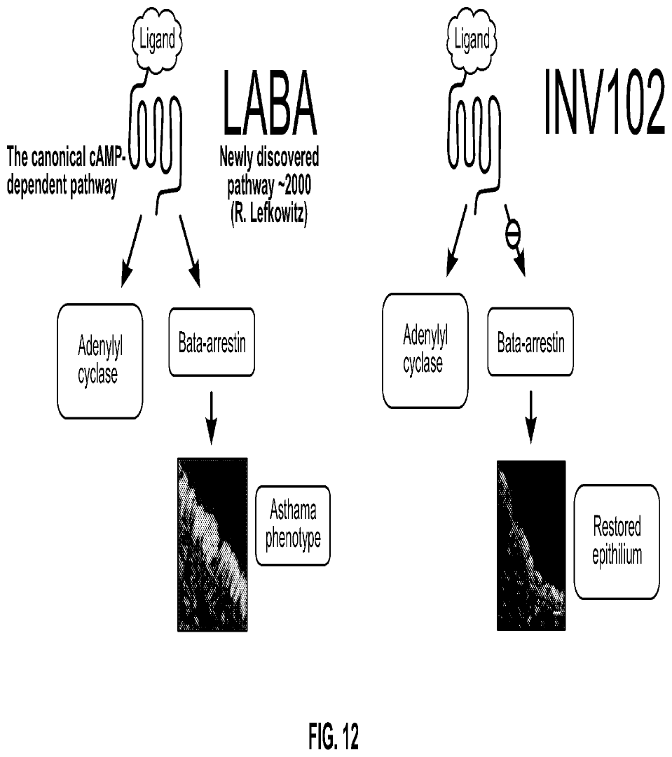

Nadolol reverses epithelial changes via inhibition of the beta-arrestin pathway in β₂-adrenergic receptors, which, in turn, results in greater therapeutic efficacy when β₂-adrenergic agonists are administered to treat diseases and conditions affecting the respiratory tract, such as asthma. This is particularly significant in treatment of short-term exacerbations of the underlying disease or condition; in diseases such as asthma, such short-term exacerbations can prove fatal. FIG. 12 is a schematic diagram showing the mechanism of action of nadolol as contrasted with the mechanism of action of long-acting β-adrenoceptor agonists (LABA) and that nadolol (“INV102”) reverses epithelial changes via inhibition of the beta-arrestin pathway in β₂ airway receptors.

### Example 13

### Nadolol Reduces the Accumulation of the Mucus-Associated Protein Mucin 5AC

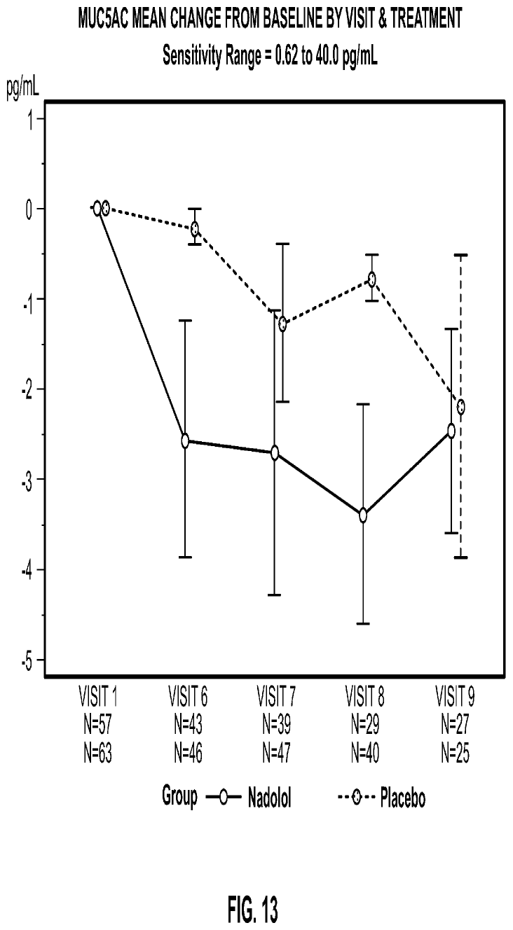

Nadolol reduces the accumulation of the mucus-associated protein mucin 5AC. FIG. 13 is a graph showing the effect of nadolol on the level of mucin 5AC in smokers treated with nadolol versus the results with a placebo. The results are statistically significant with P<0.05. After dosing ended, the phenotype regressed toward the mean, arguing for longer term treatment to enhance and maintain benefit.

### Example 14

### Administration of Nadolol Improves the Likelihood that Patients Who Attempt to Quit Smoking Will Succeed

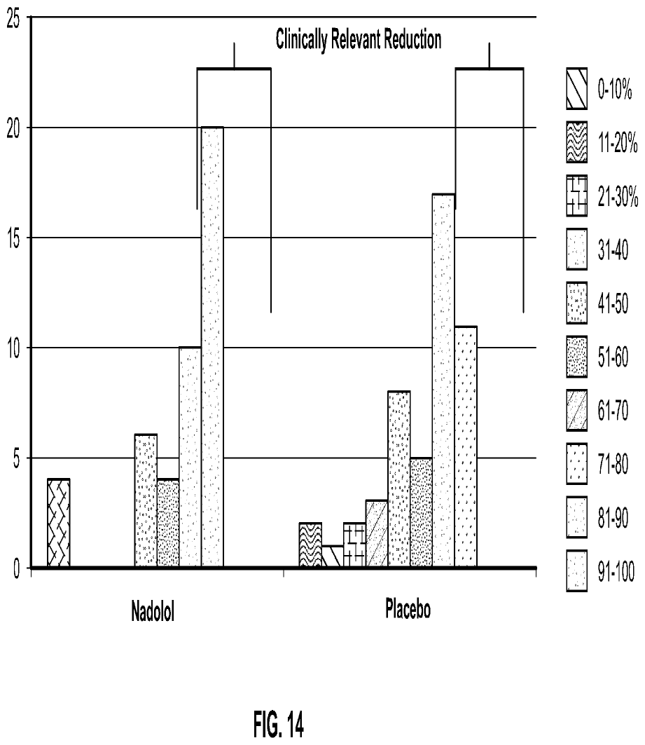

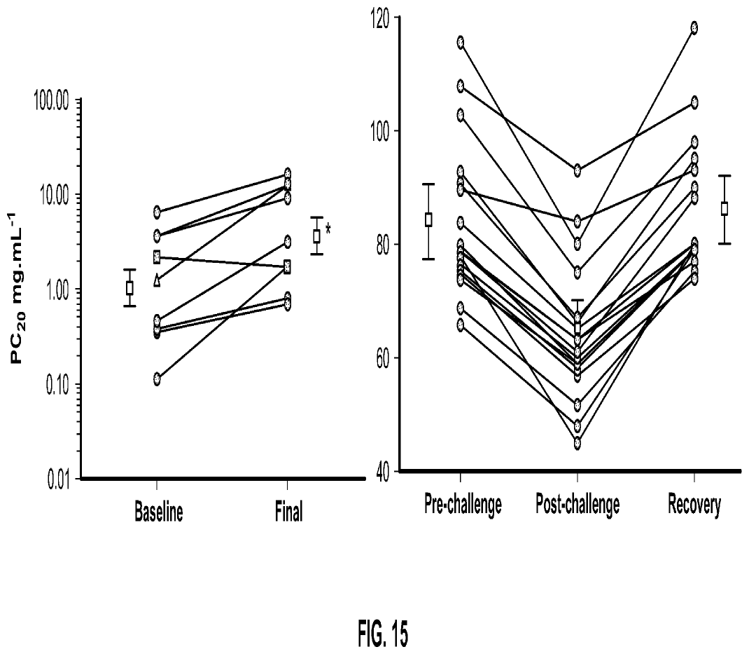

Administration of nadolol improves the likelihood that patients who attempt to quit smoking will succeed. FIG. 14 is a graph showing the effect of nadolol on the success of smoking cessation (left panel) versus a placebo (right panel); administration of nadolol produced a 70% reduction in smoking in patients who had a history of failing to quit smoking programs at least 5 times.

### Example 15

### Administration of Nadolol Does Not Block Effectiveness of the Administration of Salbutamol in Patients with Mild Asthma

Administration of nadolol does not block the effectiveness of the administration of salbutamol in patients with mild asthma as shown in FIG. 15. FIG. 15 is a set of graphs showing that nadolol does not block the effectiveness of the administration of salbutamol (2.5 mg, administered by nebulization), administered after methacholine challenge in subjects with mild asthma. Salbutamol is a short-acting β₂-adrenergic receptor agonist.

### Example 16

### Nadolol Acts to Block the Beta-Arrestin Pathway

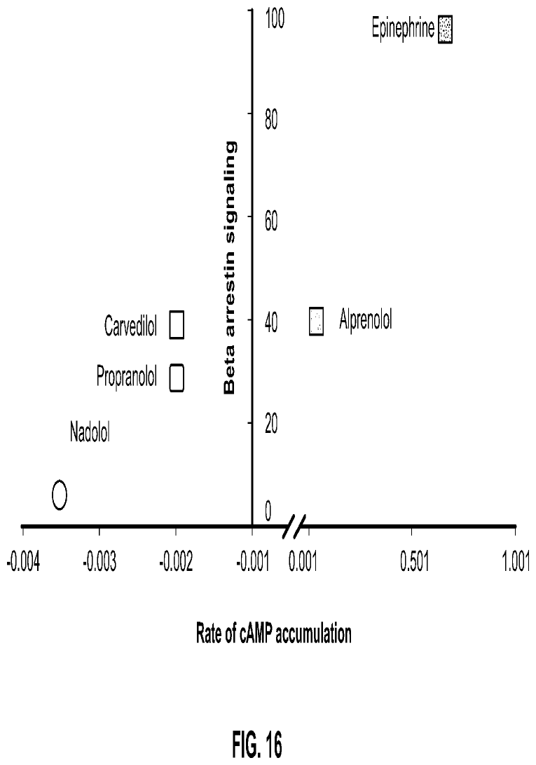

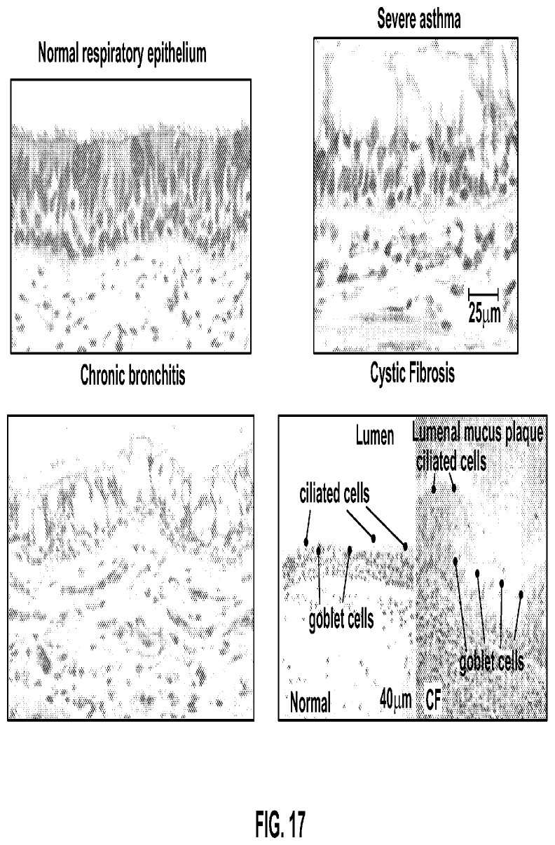

Nadolol acts to block the β-arrestin pathway, which, as stated above, results in greater therapeutic efficacy when β₂-adrenergic agonists are administered to treat diseases and conditions affecting the respiratory tract, such as asthma. In comparison, carvedilol, propranolol, and alprenolol did not block the β-arrestin pathway, as shown in FIG. 16. Beta-blockers are typically associated with adverse mucus production via the β-arrestin pathway; nadolol blocks this pathway. Nadolol, as shown in FIG. 16, is not associated with cAMP accumulation.

### Example 17

### Nadolol Can Restore the Normal State of the Respiratory Epithelium

As shown above, nadolol, through its actions in acting as a beta-adrenergic inverse agonist and inhibiting the activity of the β-arrestin pathway, can restore the normal state of the respiratory epithelium. FIG. 17 is a set of photomicrographs showing the respiratory epithelium in: normal subject without airway disease (upper left); severe asthma (upper right); chronic bronchitis (lower left); and cystic fibrosis (lower right). These diseases and conditions are among the diseases and conditions that can be treated by the administration of nadolol as described above, together with other agents, also as described above.

### Advantages of the Invention

The present invention provides improved methods and compositions for treating pulmonary airway diseases and conditions by a novel mechanism involving inhibition of arrestin-2 (β-arrestin). These methods and compositions are particularly useful in treating chronic obstructive pulmonary disease (COPD), and can also be used for treatment of pulmonary symptoms associated with infection with SARS-CoV-2, particularly in patients with COPD or other chronic respiratory conditions, These methods and compositions avoid the tolerance or tachyphylaxis that is often the consequence of therapy with conventional therapy with agents such as β-adrenergic agonists. The use of inverse agonists, in essence, forces the body to respond by improving its own signaling mechanisms to counter the pulmonary airway disease. Accordingly, compositions and methods that employ inverse agonists have broad potential for treating such diseases and conditions without the induction of tolerance. This promises superior long-term results in the treatment of such conditions without interfering with short-term acute therapy. Methods and compositions according to the present invention are well-tolerated and can be used together with other methods for treating pulmonary airway diseases and their sequelae.

As used herein in the specification and claims, the transitional phrase “comprising” and equivalent language also encompasses the transitional phrases “consisting essentially of” and “consisting of” with respect to the scope of any claims presented herein, unless the narrower transitional phrases are explicitly excluded.

Methods according to the present invention possess industrial applicability for the preparation of a medicament for the treatment of pulmonary airway diseases, including, but not limited to, chronic obstructive pulmonary disease. Methods according to the present invention also possess industrial applicability for use in treating such pulmonary airway diseases. Compositions according to the present invention possess industrial applicability as pharmaceutical compositions, particularly for the treatment of pulmonary airway diseases.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug. Methods as described herein also encompass use of the compounds, combinations of compounds, or compositions described herein for the treatment of the diseases or conditions described herein, as well as methods for the preparation of a medicament for the treatment of the diseases or conditions described herein.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

1.-168. (canceled)

169. A method for treatment of pulmonary airway disease in a subject suffering from pulmonary airway disease comprising administration of a therapeutically effective quantity of nadolol to inhibit the R-arrestin pathway to treat the pulmonary airway disease in the subject.

170. The method of claim 169 wherein the pulmonary airway disease is selected from the group consisting of chronic obstructive pulmonary disease (COPD), asthma, bronchiectasis, bronchitis, Churg-Strauss syndrome, pulmonary sequelae of cystic fibrosis, emphysema, allergic rhinitis, pneumonia, and pulmonary symptoms associated with infection with SARS-CoV-2.

171. The method of claim 170 wherein the pulmonary airway disease is chronic obstructive pulmonary disease.

172. The method of claim 169 wherein the nadolol is administered by a route selected from the group consisting of oral, sustained-release oral, parenteral, sublingual, buccal, administration by insufflation, and administration by inhalation.

173. The method of claim 169 wherein the dosage of nadolol is administered by a process of titration over time in a series of graduated doses starting from the lowest dose and increasing to the highest dose.

174. The method of claim 173 wherein, when the highest dose is reached, the nadolol continues to be administered at that dose.

175. The method of claim 169 wherein the method further comprises administration of a therapeutically effective quantity of an additional agent, wherein the additional agent is selected from the group consisting of:
(a) a β₂-selective adrenergic agonist selected from the group consisting of albuterol, arfomoterol, bambuterol, bitolterol, broxaterol, buphenine, carbuterol, clenbuterol, clorprenaline, colterol, dobutamine, fenoterol, formoterol, isoetharine, isoprenaline, levabuterol, levosalbutamol, mabuterol, metaprotenerol, methoxyphenamine, pirbuterol, procaterol, ractopamine, reproterol, ritodrine, salmeterol, terbutaline, zilpaterol, and the salts, solvates, and prodrugs thereof;
(b) a corticosteroid selected from the group consisting of AZD-5423 (2,2,2-trifluoro-N-[(1 R,2S)-1-{[1-(4-fluorophenyl)-1H-indazol-5-yl]oxy}-1-(3-methoxyphenyl)-2-propanyl]acetamide), beclomethasone, budesonide, ciclesonide, deflazacort, flunisolide, fluticasone, methylprednisolone, mometasone, prednisolone, prednisone, dexamethasone, and triamcinolone, and the salts, solvates, and prodrugs thereof;
(c) an anticholinergic drug selected from the group consisting of ipratropium bromide, tiotropium bromide, oxitropium bromide, abediterol, aclidinium bromide, glycopyrronium bromide, umeclidinium bromide, and the salts, solvates, and prodrugs thereof;
(d) a xanthine compound selected from the group consisting of theophylline, extended-release theophylline, aminophylline, theobromine, enprofylline, diprophylline, isbufylline, choline theophyllinate, albifylline, arofylline, bamifylline, caffeine, 8-chlorotheophylline, diprophylline, doxofylline, enprofylline, etamiphylline, furafylline, 1-isobutyl-1-methylxanthine, proxyphylline, and xanthinol, and the salts, solvates, and prodrugs thereof;
(e) an anti-IgE antibody;
(f) a leukotriene antagonist selected from the group consisting of montelukast, pranlukast, and zafirlukast, and the salts, solvates, and prodrugs thereof;
(g) a phosphodiesterase IV inhibitor selected from the group consisting of roflumilast, cilomilast, piclamilast, and ibudilast, and the salts, solvates, and prodrugs thereof;
(h) a 5-lipoxygenase inhibitor selected from the group consisting of zileuton and fenleuton, and the salts, solvates, and prodrugs thereof;
(i) a mast cell stabilizer selected from the group consisting of azelastine, cromoglicic acid, ketotifen, lodoxamide, nedocromil, olopatadine, and pemirolast, and the salts, solvates, and prodrugs thereof; and
(j) a biological selected from the group consisting of an anti-IL4 antibody, an anti-IL13 antibody, an inhibitor of both IL4 and IL13, an anti-IL5 antibody, and an anti-IL8 antibody.

176. The method of claim 169 wherein the method further comprises administration of a therapeutically effective quantity of an arrestin-2 inhibitor, wherein the arrestin-2 inhibitor is selected from the group consisting of:
(a) a protein fragment of arrestin-2;
(b) a compound of Formula (A-I);
(c) an omega-3 fatty acid selected from the group consisting of DHA (docosahexaenoic acid) and EPA (eicosapentaenoic acid);
(d) a CXCR2 inhibitor selected from the group consisting of SB225002 (N-(2-bromophenyl)-N′-(2-hydroxy-4-nitrophenyl)urea), AZD5069 (N-(2-((2,3-difluorobenzyl)thio)-6-(((2R,3S)-3,4-dihydroxybutan-2-yl)oxy)pyrimidin-4-yl)azetidine-1-sulfonamide); SB265610 (1-(2-bromophenyl)-3-(4-cyano-1H-benzo[d][1,2,3]triazol-7-yl)urea); navarixin; danirixin; CXCR2-IN-1 (1-(2-chloro-3-fluorophenyl)-3-[4-chloro-2-hydroxy-3-(1-methylpiperidin-4-yl)sulfonylphenyl]urea); SRT3109 (N-(2-((2,3-difluorobenzyl)thio)-6-((3,4-dihydroxybutan-2-yl)amino)pyrimidin-4-yl)azetidine-1-sulfonamide); and SRT3190 (N-[2-[(2,3-difluorophenyl)methylsulfanyl]-6-[[(2S,3R)-3,4-dihydroxybutan-2-yl]amino]pyrimidin-4-yl]azetidine-1-sulfonamide);
(e) a MyD88 inhibitor selected from the group consisting of ST2825 ((4R,7R,8aR)-1′-[2-[4-[[2-(2,4-dichlorophenoxy)acetyl]amino]phenyl]acetyl]-6-oxospiro[3,4,8,8a-tetrahydro-2H-pyrrolo[2,1-b][1,3]thiazine-7,2′-pyrrolidine]-4-carboxamide) and T6167923 (4-(3-bromophenyl)sulfonyl-N-(1-thiophen-2-ylethyl)piperazine-1-carboxamide); and
(f) a MD2 inhibitor.

177. The method of claim 169 wherein the method further comprises administration of a therapeutically effective quantity of an inhibitor of a GRK, wherein the inhibitor of a GRK is selected from the group consisting of:
(a) a nitric oxide donor that donates nitric oxide or a related redox species;
(b) inositol hexaphosphate;
(c) barbadin;
(d) an inhibitor of protein kinase A; and
(e) a phospholipase C inhibitor.

178. A pharmaceutical composition comprising:
(a) a therapeutically effective quantity of nadolol to inhibit the R-arrestin pathway to treat a pulmonary airway disease; and
(b) a pharmaceutically acceptable carrier;
wherein administration of the pharmaceutical composition treats the pulmonary airway disease.

179. The pharmaceutical composition of claim 178 wherein the pulmonary airway disease is selected from the group consisting of chronic obstructive pulmonary disease (COPD), asthma, bronchiectasis, bronchitis, Churg-Strauss syndrome, pulmonary sequelae of cystic fibrosis, emphysema, allergic rhinitis, pneumonia, and pulmonary symptoms associated with infection with SARS-CoV-2.

180. The pharmaceutical composition of claim 178 wherein the composition is formulated for administration by a route selected from the group consisting of oral, sustained-release oral, parenteral, sublingual, buccal, administration by insufflation, and administration by inhalation.

181. The pharmaceutical composition of claim 178 wherein the composition further comprises a therapeutically effective quantity of an additional therapeutic agent, wherein the additional agent is selected from the group consisting of:
(a) a β₂-selective adrenergic agonist selected from the group consisting of albuterol, arfomoterol, bambuterol, bitolterol, broxaterol, buphenine, carbuterol, clenbuterol, clorprenaline, colterol, dobutamine, fenoterol, formoterol, isoetharine, isoprenaline, levabuterol, levosalbutamol, mabuterol, metaprotenerol, methoxyphenamine, pirbuterol, procaterol, ractopamine, reproterol, ritodrine, salmeterol, terbutaline, zilpaterol, and the salts, solvates, and prodrugs thereof;
(b) a corticosteroid selected from the group consisting of AZD-5423 (2,2,2-trifluoro-N-[(1 R,2S)-1-{[1-(4-fluorophenyl)-1H-indazol-5-yl]oxy}-1-(3-methoxyphenyl)-2-propanyl]acetamide), beclomethasone, budesonide, ciclesonide, deflazacort, flunisolide, fluticasone, methylprednisolone, mometasone, prednisolone, prednisone, dexamethasone, and triamcinolone, and the salts, solvates, and prodrugs thereof;
(c) an anticholinergic drug selected from the group consisting of ipratropium bromide, tiotropium bromide, oxitropium bromide, abediterol, aclidinium bromide, glycopyrronium bromide, umeclidinium bromide, and the salts, solvates, and prodrugs thereof;
(d) a xanthine compound selected from the group consisting of theophylline, extended-release theophylline, aminophylline, theobromine, enprofylline, diprophylline, isbufylline, choline theophyllinate, albifylline, arofylline, bamifylline, caffeine, 8-chlorotheophylline, diprophylline, doxofylline, enprofylline, etamiphylline, furafylline, 1-isobutyl-1-methylxanthine, proxyphylline, and xanthinol, and the salts, solvates, and prodrugs thereof;
(e) an anti-IgE antibody;
(f) a leukotriene antagonist selected from the group consisting of montelukast, pranlukast, and zafirlukast, and the salts, solvates, and prodrugs thereof;
(g) a phosphodiesterase IV inhibitor selected from the group consisting of roflumilast, cilomilast, piclamilast, and ibudilast, and the salts, solvates, and prodrugs thereof;
(h) a 5-lipoxygenase inhibitor selected from the group consisting of zileuton and fenleuton, and the salts, solvates, and prodrugs thereof;
(i) a mast cell stabilizer selected from the group consisting of azelastine, cromoglicic acid, ketotifen, lodoxamide, nedocromil, olopatadine, and pemirolast, and the salts, solvates, and prodrugs thereof; and
(j) a biological selected from the group consisting of an anti-IL4 antibody, an anti-IL13 antibody, an inhibitor of both IL4 and IL13, an anti-IL5 antibody, and an anti-IL8 antibody.

182. The pharmaceutical composition of claim 178 wherein the composition further comprises a therapeutically effective quantity of an arrestin-2 inhibitor, wherein the arrestin-2 inhibitor is selected from the group consisting of:
(a) a protein fragment of arrestin-2;
(b) a compound of Formula (A-I);
(c) an omega-3 fatty acid selected from the group consisting of DHA (docosahexaenoic acid) and EPA (eicosapentaenoic acid);
(d) a CXCR2 inhibitor selected from the group consisting of SB225002 (N-(2-bromophenyl)-N′-(2-hydroxy-4-nitrophenyl)urea), AZD5069 (N-(2-((2,3-difluorobenzyl)thio)-6-(((2R,3S)-3,4-dihydroxybutan-2-yl)oxy)pyrimidin-4-yl)azetidine-1-sulfonamide); SB265610 (1-(2-bromophenyl)-3-(4-cyano-1H-benzo[d][1,2,3]triazol-7-yl)urea); navarixin; danirixin; CXCR2-IN-1 (1-(2-chloro-3-fluorophenyl)-3-[4-chloro-2-hydroxy-3-(1-methylpiperidin-4-yl)sulfonylphenyl]urea); SRT3109 (N-(2-((2,3-difluorobenzyl)thio)-6-((3,4-dihydroxybutan-2-yl)amino)pyrimidin-4-yl)azetidine-1-sulfonamide); and SRT3190 (N-[2-[(2,3-difluorophenyl)methylsulfanyl]-6-[[(2S,3R)-3,4-dihydroxybutan-2-yl]amino]pyrimidin-4-yl]azetidine-1-sulfonamide);
(e) a MyD88 inhibitor selected from the group consisting of ST2825 ((4R,7R,8aR)-1′-[2-[4-[[2-(2,4-dichlorophenoxy)acetyl]amino]phenyl]acetyl]-6-oxospiro[3,4,8,8a-tetrahydro-2H-pyrrolo[2,1-b][1,3]thiazine-7,2′-pyrrolidine]-4-carboxamide) and T6167923 (4-(3-bromophenyl)sulfonyl-N-(1-thiophen-2-ylethyl)piperazine-1-carboxamide); and
(f) a MD2 inhibitor.

183. The pharmaceutical composition of claim 178 wherein the composition further comprises a therapeutically effective quantity of an inhibitor of a GRK, wherein the inhibitor of a GRK is selected from the group consisting of:
(a) a nitric oxide donor that donates nitric oxide or a related redox species;
(b) inositol hexaphosphate;
(c) barbadin;
(d) an inhibitor of protein kinase A; and
(e) a phospholipase C inhibitor.

184. The pharmaceutical composition of claim 178 wherein the pharmaceutically acceptable carrier is selected from the group consisting of a solvent, a dispersion medium, a coating, an antibacterial agent, an antifungal agent, an isotonic agent, an absorption delaying agent, a preservative, a sweetening agent for oral administration, a thickening agent, a buffer, a liquid carrier, a wetting, solubilizing, or emulsifying agent; an acidifying agent, an antioxidant, an alkalinizing agent, a carrying agent, a chelating agent, a colorant, a complexing agent, a suspending or viscosity-increasing agent, a flavor or perfume, an oil, a penetration enhancer, a polymer, a stiffening agent, a protein, a carbohydrate, a bulking agent, and a lubricating agent.

185. The method of claim 170 wherein the pulmonary airway disease is pulmonary symptoms associated with infection with SARS-CoV-2.

186. The method of claim 172 wherein the nadolol is administered by a route selected from the group consisting of oral and sustained-release oral.

187. The composition of claim 179 wherein the pulmonary airway disease is COPD.

188. The composition of claim 179 wherein the pulmonary airway disease is pulmonary symptoms associated with infection with SARS-CoV-2.

189. The composition of claim 180 wherein the composition is formulated for administration by a route selected from the group consisting of oral and sustained-release oral.

190. The composition of claim 178 wherein the therapeutically effective quantity of nadolol per unit dose of the composition is selected from the group consisting of 1 mg, 3 mg, 5 mg, 10 mg, 15 mg, 30 mg, 50 mg, and 70 mg per unit dose.
